(19) United States
(12) Patent Application Publication
(10) Pub. No.: US 2024/0110144 A1
(43) Pub. Date: Apr. 4, 2024

(54) DELIVERY PLATFORM

(71) Applicant: Avectas Limited, Co. Kildare (IE)

(72) Inventors: Michael Maguire, Dublin (IE); Shirley O'Dea, Maynooth (IE)

(21) Appl. No.: 18/263,359

(22) PCT Filed: Feb. 1, 2022
(86) PCT No.: PCT/IB2022/050876

§ 371 Date: Jul. 28, 2023

Related U.S. Application Data
(60) Provisional application No. 63/144,136, filed on Feb. 1, 2021.

(51) Int. Cl. C12M 1/26; C12M 1/00; C12M 1/12; C12M 1/36; C12M 1/42; C12M 3/06

(52) U.S. Cl. CPC C12M 33/04; C12M 25/02; C12M 27/16; C12M 29/06; C12M 35/04; C12M 41/48

(57) ABSTRACT

The current subject matter provides a cell engineering platform for vector-free and/or viral delivery of payload/cargo compounds and compositions into cells. The platform achieves delivery to cells quickly and in an easy to use manner. Related apparatus, systems, techniques, articles and compositions are also described.

### CROSS-REFERENCE TO RELATED APPLICATIONS

This application claims priority under 35 U.S.C. § 119 to U.S. Provisional Patent Application No. 63/144,136, filed Feb. 1, 2020, entitled “Reversible Permeabilization Platform”. The entire contents of which is hereby expressly incorporated by reference herein in its entirety.

### TECHNICAL FIELD

The subject matter described herein relates to a cell engineering platform utilizing solution-based intracellular delivery.

### BACKGROUND

Variability in intracellular delivery efficiency exists among different cell types and intracellular delivery methods. Obtaining sufficient quantities of viable cells following intracellular delivery can require large scale cell engineering platforms, which can be costly to operate and require of greater quantities of target cells and reagent media. Rapidly generating high-quality, repeatable experimental data from reversibly permeabilizing smaller quantities of target cells can be time-consuming and manually intensive using conventional systems and methods.

### SUMMARY

In an aspect, a method includes filling a pod of a cell engineering platform with a mixture of cells and a first medium; and discharging the first medium from the pod through a filter, leaving the cells deposited on the filter. The cell engineering platform includes an atomizer; and a pod holder configured to receive the pod. The pod includes a filter plate and an upper portion forming a well for holding cells and media.

One or more of the following features can be included in any feasible combination. For example, a delivery solution that contains a permeabilization agent and a payload can be sprayed onto the cells deposited on the filter. A stop solution can be applied. The pod can be filled with a second medium to resuspend the cells from the filter. The discharged first medium can be reused as the second medium. The pod can be agitated. The resuspended cells can be extracted from the pod. The filling of the pod can be performed automatically with a pump and a controller. The cells can be cultured within the pod. Discharging the first medium from the pod can be performed by supplying a vacuum to the bottom of the pod. The discharging of the first medium from the pod can be performed by gravity. The applying the stop solution can be performed to wash the cells. The filling the pod with the second medium can be performed as at least one of a cell wash process, a cell concentration change process, and a cell medium change process.

The pod can include a lower portion releasably coupled to the filter plate. The pod can include a memory storing data characterizing at least one process parameter. The at least one process parameter can be read from the memory and by a controller of the cell engineering platform. At least one processing step utilizing the at least one processing parameter can be performed. The pod can include a memory storing data characterizing an experiment identifier.

In another aspect, a system includes a housing including a pod holder configured to receive a pod, the pod including a filter plate and an upper portion forming a well; a delivery solution applicator configured to deliver atomized delivery solution to the well; a display; and a controller including circuitry configured to display at least one process parameter.

One or more of the following features can be included in any feasible combination. For example, the pod holder can be configured to tilt or vibrate the pod. The delivery solution applicator can include a spray head. The pod can be sized to hold less than 1×10⁷ T cells. The system can be configured to automatically apply an atomized delivery solution to a cellular monolayer formed on a filter within the pod. The delivery solution applicator can include a nebulizer. The delivery solution applicator can further include a mass flow controller or a volumetric flow controller to regulate a gas flow to operate the nebulizer. The delivery solution applicator is configured to deliver 10⁻³⁰⁰ micro liters of the delivery solution per actuation.

The delivery solution can include an aqueous solution, the aqueous solution including the payload and an alcohol at greater than 2 percent (v/v) concentration. The alcohol can include ethanol. The aqueous solution can include greater than 5% ethanol. The aqueous solution can include between 5-30% ethanol. The aqueous solution can include 12% or 25% ethanol. The aqueous solution can include between 12.5-500 mM KCl. The aqueous solution can include 106 mM KCl. The well can be configured to contain a population of non-adherent cells. The non-adherent cell can include a peripheral blood mononuclear cell. The non-adherent cell can include an immune cell. The non-adherent cell can include a T lymphocyte. The payload can include a messenger ribonucleic acid (mRNA). The mRNA can encode a gene-editing composition. The gene editing composition can reduce the expression of PD-1. The mRNA can encode a chimeric antigen receptor.

The system can be used to deliver a cargo compound or composition to a mammalian cell. The population of non-adherent cells can include a monolayer.

In another aspect, a system includes a housing including a base, at least one controller including circuitry configured to control an operation of the system, and a display. The system further includes one or more fluid circuits including at least one valve, at least one pump, a syringe, and at least one fluid detection sensor; a chamber assembly received within an articulating frame extending from the front surface of the housing, wherein the chamber assembly is sealed from atmospheric conditions in operation and includes a filter; at least one media container; at least one cell culture container fluidically coupled to the chamber assembly via the one or more fluid circuits; and at least one collection tray configured to receive media or cells.

One or more of the following features can be included in any feasible combination. For example, the articulating frame can be configured to agitate the chamber assembly. The chamber assembly can include a memory storing data characterizing at least one process parameter. The at least one controller can be configured to read, via the circuitry, the data characterizing the at least one process parameter from the memory, and perform, via the circuitry, at least one processing step utilizing the at least one processing parameter. The chamber assembly can include a memory storing data characterizing an experiment identifier.

In some implementations, the operation of the system can include at least one of a cell wash process, a cell concentration change process, and a cell medium change process. The display can include a human-machine interface configured to receive inputs associated with the operation of the system. The articulating frame can articulate to an angle between 0-10.0, 10.1-15.0, 15.1-20.0, 20.1-25.0, 25.1-30.0, 30.1-35.0, 35.1-40.0, or 40.1-45.0 degrees with respect to a horizontal surface on which the system is positioned. The articulating frame can oscillate between two angles at a predetermined or user-defined frequency. The predetermined or user-defined frequency can be between 0-0.5 kHz, 0.51-1.0 kHz, 1.1-1.5 kHz, 1.51-2.0 kHz, 2.01-2.5 kHz, or greater than 2.51 kHz. The at least one collection tray can include a cooling element or a heating element.

In some implementations, the base can include a scale positioned below the at least one collection tray. The at least one fluid detection sensor can be arranged with respect to at least one fluidic circuit of the one or more fluidic circuits. A first fluid detection sensor can be configured at a first location of the at least one fluidic circuit and a second fluid detection sensor can be configured at a second location of the at least one fluidic circuit. The first fluid detection sensor and the second fluid detection sensor can be operable to calculate a volume of the media between the first location and a second location of the at least one fluidic circuit. The at least one pump can be a peristaltic pump. The system can include a syringe holder to hold the syringe. The syringe holder can include an optical sensor configured to determine a level of fluid within the syringe or a position of a plunger of the syringe. The optical sensor can include an array of a plurality of optical sensors.

In some implementations, the system can include at least one electrical connector configured to communicatively couple an instrument to the system. The instrument can include at least one of an thermometer, a hydrometer, a barometer, a photoplethysmograph sensor, a load cell, a biochemical sensor, an optical sensor, a transducer, or a microelectronic machine. The system can include at least one first gas connector coupling a first gas supply to the chamber assembly via a first gas circuit. The system includes a second gas connector coupling a second gas supply to the chamber assembly via a second gas circuit, the second connector configured to operate independently of the at least one first gas connector. The system can include at least one hanger configured to position a source of the media above the chamber assembly. The hanger can include a scale configured within the hanger to determine a weight of the source of the media. The system can includes a bar code reader. The system can include a tube welder. The system can includes an insulative jacket or a conductive jacket at least partially enclosing the chamber assembly.

An inner surface of the chamber assembly can include a coating or a pattern configured to aid cell mobility or adherence to the inner surface. The chamber assembly can include an upper portion removable from a lower portion, the lower portion including the filter. The filter can include a coating or a pattern configured to aid cell mobility or adherence to the filter. The upper portion can include a gas port at which a gas can be received from the first gas circuit. The upper portion can include an air diffuser opening and an air diffuser positioned within the air diffuser opening, the air diffuser coupled to the second gas circuit. The upper portion can include a spray head opening and a spray head positioned within the spray head opening. The spray head can include a gas inlet port coupled to the first gas circuit and a fluid inlet port coupled to a supply of an isotonic aqueous solution including a payload and an alcohol.

The at least one controller can be configured to control one or more of a pressure, a temperature, and a gas composition within the chamber assembly. The gas composition can include at least one of carbon dioxide, nitrogen, or oxygen. The chamber assembly can include a heating element. The system can be configured for use as a bioreactor for incubating cells. The system can be configured for use in a cell cryopreservation process. The system can be configured for use in a cell permeabilization process. The system can be configured for use in a cell transduction process. The system can be configured for use in a cell transfection process.

In yet another aspect, a device for use to deliver a cargo to cells in the absence of alcohol is provided. The device includes a housing including a base, at least one controller including circuitry configured to control an operation of the device, and a display. The system further includes one or more fluid circuits including at least one valve, at least one pump, a syringe, and at least one fluid detection sensor; a chamber assembly received within an articulating frame extending from the front surface of the housing, wherein the chamber assembly is sealed from atmospheric conditions in operation and includes a filter; at least one media container; at least one cell culture container fluidically coupled to the chamber assembly via the one or more fluid circuits; and at least one collection tray configured to receive media or cells.

The details of one or more variations of the subject matter described herein are set forth in the accompanying drawings and the description below. Other features and advantages of the subject matter described herein will be apparent from the description and drawings, and from the claims.

### DESCRIPTION OF DRAWINGS

FIG. 1 is an isometric view of a computer aided design (CAD) drawing illustrating an example embodiment of a delivery platform according to some embodiments disclosed herein.

FIG. 2A is a side view of the delivery platform shown in FIG. 1.

FIG. 2B is a front view of the delivery platform shown in FIG. 1.

FIG. 3 is a side view of another example embodiment of the delivery platform shown in FIG. 1, according to some embodiments discloses herein.

FIG. 4A is an isometric view of a CAD drawing illustrating an example embodiment of a base assembly of the delivery platform shown in FIG. 1.

FIG. 4B is a pneumatic diagram of some implementations of the platform shown in FIG. 1.

FIG. 5 is an isometric view of a CAD drawing illustrating an example embodiment of a spine assembly of the delivery platform shown in FIG. 1.

FIG. 6A-B is an isometric view of a CAD drawing illustrating an example embodiment of a top assembly of the delivery platform shown in FIG. 1.

FIGS. 7A-7E are CAD drawings illustrating an example Eppendorf base support of the delivery platform of FIG. 1.

FIGS. 8A-8E are CAD drawings illustrating an example upper mount of a clippard module of the delivery platform of FIG. 1.

FIGS. 9A-9H are CAD drawings illustrating an example lower mount of a clippard module of the delivery platform of FIG. 1.

FIG. 10A-G illustrates example atomizers for use in the delivery platform of FIG. 1.

FIGS. 11A-11E are CAD drawings illustrating an example spray head base mounting platform of the delivery platform of FIG. 1.

FIGS. 12A-12D are CAD drawings illustrating an upper portion of an exemplary embodiment of a pod locating nest of the delivery platform of FIG. 1.

FIGS. 13-13C are CAD drawings illustrating a lower portion of an exemplary embodiment of a pod locating nest of the delivery platform of FIG. 1.

FIGS. 14A-14F are CAD drawings illustrating an example pod nest cover of the delivery platform of FIG. 1.

FIG. 15 is an image of an example embodiments of a pod assembly for use in the delivery platform shown in FIG. 1.

FIGS. 16A-16C are images of example embodiments of components of the pod assembly shown in FIG. 15.

FIG. 16D is another example embodiment of the pod assembly of FIG. 15.

FIG. 17 is an isometric view of a CAD drawing illustrating an exemplary embodiment of a pod assembly within a pod nest of the delivery platform of FIG. 1.

FIG. 18 is an cross-sectional view of the exemplary embodiment shown in FIG. 17.

FIGS. 19A-19C are CAD drawings illustrating example embodiments of a filter plate coupling of the pod assembly of FIG. 15.

FIG. 20 is a flow diagram illustrating an example embodiment of a process for delivery to cells using the delivery platform of FIG. 1.

FIG. 21A-B illustrate example frames for stacking and processing pods.

FIG. 22 illustrates an example spray-guard according to some example implementations.

FIG. 23 illustrates an image of another example embodiment of a delivery platform according to some embodiments disclosed herein.

FIG. 24 illustrates a view of the platform shown in FIG. 23.

FIG. 25 illustrates a second view of the platform shown in FIG. 23.

FIG. 26 illustrates a close-up view of a portion of the platform shown in FIG. 23.

FIG. 27 illustrates an image of an example embodiment of a single-use assembly of the delivery platform shown in FIG. 23.

FIG. 28 illustrates an image of an example embodiment of a spray head of the single-use assembly shown in FIG. 27.

FIG. 29 illustrates a schematic of the experimental design for simultaneous delivery of RNPs. Cas9 RNP—TRAC sgRNA was prepared at 2:1 ratio at 0.4 μg/μL (equiv to 3.3 μg per 1×10⁶ cells); S Buffer solutions were prepared with 0, 5, 10 and 15% ethanol with RNP and the experiments were carried out on the SOLUPORE® delivery system with the S buffer solutions at each ethanol concentration.

FIG. 30 illustrates representative flow cytometry plots from cells stained with an antibody targeting CD3 (gated off the live population). Untreated (UT) cells showed >93% positivity for CD3 and this was reduced following delivery of TRAC RNP by the example delivery platform illustrated with respect to FIG. 1. Two distinct populations are observed in the treated samples with the population on the left (gated) referring to those cells that were negative for CD3 staining. This negative population increased from ˜59% in samples where no ethanol was present in the delivery Solution to ˜67% in samples where ethanol was present. A limit exists to the amount of ethanol present before precipitation of the Cas9 protein occurs (>20% ethanol at 0.4 μg/μL Cas9 RNP).

FIG. 31A is a bar graph showing the mean CD3 negative population (±standard deviation) from 2-3 replicates per condition in activated T cells 72 hr post-delivery of TRAC RNP (2:1 guide to Cas9 molar ratio; 3.3 μg per 1×10⁶ cells) by the example delivery platform illustrated with respect to FIG. 1. Increasing concentrations of ethanol were added with the cargo in the delivery solution. The level of CD3 edit increased modestly with increasing concentrations of ethanol (0% EtOH-58% to 15% EtOH-66%). “UT” refers to untreated control cells.

FIG. 31B is a table showing the mean, standard deviation, standard error of the mean and coefficient of variation of CD3 negative expression from each group 72 hr post-delivery of TRAC RNP by the example delivery platform illustrated with respect to FIG. 1.

FIG. 32 is a bar graph depicting the percent viability at the increasing ethanol concentrations, and time points consisting of pre-delivery, post-delivery (day 3) and post-delivery (day 5).

FIG. 33A is a line graph showing that aqueous solutions without ethanol show a larger droplet size for the same pressure as compared to a solution containing ethanol. As shown in the graph, for achieving spray particle size similar to cells of approximately 10 μm in diameter (e.g., human T cells), the spray droplet size requires higher atomization pressures to be applied to maintain the droplet size range closer to the cell size, including to avoid excessively large droplets.

FIG. 33B is a line graph showing that aqueous solutions with ethanol show a smaller droplet size (as compared to aqueous solutions without ethanol for the same pressure).

FIG. 34A and FIG. 35B are bar graphs showing that an increase in GFP transfection was achieved using 12% ethanol in solutions and increasing the proportions of sucrose and sodium chloride from the two buffer solutions. The cell viability was also maintained.

FIG. 35A and FIG. 35B are bar graphs showing that an increase in GFP transfection was achieved using 27% ethanol in solutions and increasing the proportions of sucrose and sodium chloride from the two buffer solutions. The cell viability was also maintained. Like reference symbols in the various drawings indicate like elements.

FIG. 36 is a line graph showing a linear regression analysis demonstrating that the osmolal gap was solely due to ethanol, based on the difference between measured serum osmolality after ethanol addition and measured serum osmolality before ethanol addition and serum ethanol concentration in mg/dL. Osmolal Gap (mOsm/kg H2O)=0.234 (Ethanol [mg/dL])−1.427 (95% CI: slope 0.226-0.243, intercept −2.971 to 0.118). FIG. 36 is reproduced from Nguyen, M. et al “Front. Med. Is the Osmolal Concentration of Ethanol Greater Than Its Molar Concentration? Jan. 8, 2020, “Nguyen” incorporated herein by reference in its entirety).

FIG. 37 is a bar graph showing that hypertonic solutions increase transfection.

FIG. 38 is a bar graph showing the effect of the hypertonic solutions on viability.

### DETAILED DESCRIPTION

Despite some advances, delivery of certain particles and/or molecules into cells remains a challenge. Factors such as size or charge of a molecule to be delivered into a cell can limit and/or prevent delivery of the molecule into the cell. In particular, delivery across the cell membrane can be complicated due to the molecule and/or the membrane of the cell. A cell membrane or plasma is a semi-permeable biological membrane, which acts as a selective barrier. The membrane regulates an internal chemical composition of the cell. As the selective barrier for the cell, the membrane can allow only certain molecules to passively translocate across the membrane through, for example, passive diffusion into the cell. Small, hydrophobic molecules (such as O₂, CO₂, and N₂) and small, uncharged polar molecules (such as H₂O and glycerol) can passively diffuse across cell membranes. Larger, uncharged polar molecules (such as amino acids, glucose, and nucleotides) and ions (such as H⁺, Na⁺, K⁺ and Cl⁻) cannot passively diffuse across cell membranes.

Reversible permeabilization can be used for intracellular delivery of compounds in clinical settings, as well as in research and development environments. For example, in clinical or therapeutic treatment methods, cells can be extracted from a patient, isolated (e.g., concentrated or enriched), and subsequently be treated with the cell engineering methods. The engineered cells can be expanded and returned to the patient. For delivery across cell membranes, methods using viral vectors can be used. However, the methods based on viral vectors generally require high costs and complex processes, provide limited accessibility, and offer variable and inconsistent results. Methods based on electroporation can also be used. However, the electroporation-based methods generally result in higher cell damage and offer poor cell recovery and cell functionality.

An object of the present disclosure is to provide solution-based delivery to address the cost and complexity challenges for the cell engineering technologies. To provide a reliable and consistent method for cell therapies, the current subject matter can provide a cell engineering method and platform to deliver compounds or mixtures of compounds (e.g., payload) into cells across cell membranes by contacting the cells with a delivery solution containing the payload. The cells may be suspension cells or adherent. In some implementations, the delivery of payload into cells across cell membranes can be performed by including in the solution an agent for reversibly permeabilizing cell membranes, which can also be referred to as a cell poration process. Further, poration of cells can refer to a process of permeabilizing cell membranes and delivering payloads across cell membranes into cells.

Some implementations of the current subject matter can provide a platform for cell engineering that can provide clinical grade transfection in that treated cells have high viability and expression. In addition, the delivery platform can provide smaller scale cell processing and can be used for experimental designs involving smaller quantities of cells, such as 0.5M-15M cells. The platform can include features that make it easy to use, for example, by having single-use pods for performing the cell engineering process that is described in more detail herein. In some embodiments, the pod can be resuable. In some embodiments, the pods can be chamber. The system can include control features enabling easy to implement and repeatable cell processing. Some implementations can be particularly useful, for example, in research and development efforts. The platform can also be used for vector-free delivery of payload/cargo compounds and compositions into non-adherent cells.

Using the platform of the present disclosure, other cell engineering processes may also be performed before and/or after the delivery process, which can significantly enhance productivity and allow the overall process to be streamlined. Moreover, not only the non-viral transfection method but also viral methods may be performed within the single platform.

The delivery platform described herein can achieve delivery of a payload across a plasma membrane of a non-adherent cell by performing the steps of providing a population of non-adherent cells and contacting the population of cells with a volume of an isotonic aqueous solution, the aqueous solution including the payload. In some implementations, the aqueous solution does not include an alcohol (e.g., the solution is in the absence of alcohol (e.g., 0% ethanol)). In some implementations, the solution can also include an alcohol at greater than 0.2 percent (v/v) concentration. For example, the alcohol comprises ethanol (e.g., greater than 5% ethanol, greater than 10% ethanol, and the like). In some examples, the aqueous solution comprises between 20-30% ethanol, e.g., 27% ethanol. Other compositions are possible.

The current subject matter can also provide a platform that can automate the cell poration process and allow delivery to cells to be performed at a various scales. When cells are manually loaded to the platform and/or manually unloaded from the platform, the throughput of the system is limited, difficulties exist in applying to clinical process/treatment. There may be concerns for contamination and inconsistent process depending on operators and/or various environmental parameters. By the process automation, the delivery process can be performed more consistently, a concern for contamination can be significantly reduced, and therefore, the system can be scaled more easily. Exemplary embodiments of the delivery platform to perform the delivery process with manual and automated processes will be described.

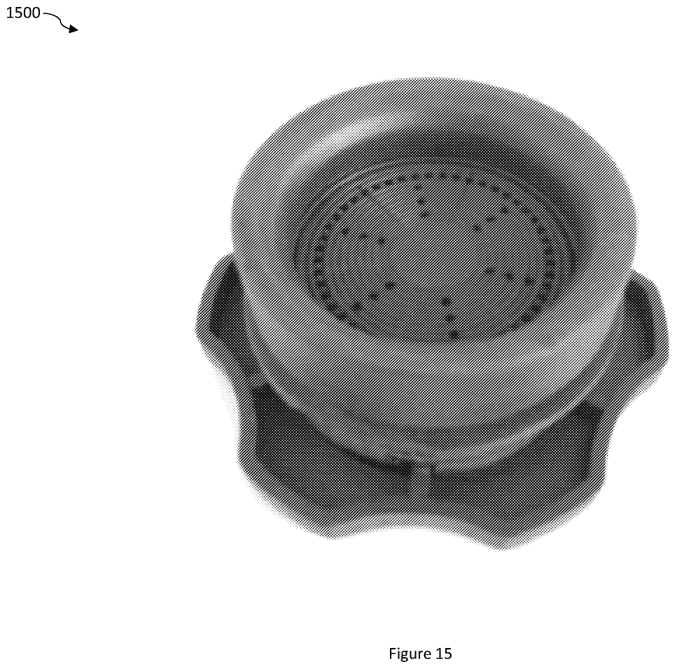

An example pod according to some implementations is shown in FIGS. 15-19 and is described more fully below. The example pod includes an upper portion 1605, a filter plate 1610, and a lower portion 1615. In some implementations, pods may be designed for specific cell populations and sizes. For example, pods can include different lower portions based on the culture. As used herein, the pod can be referred to as a chamber, a chamber assembly, a single-use assembly, or a disposable assembly, for example.

In some implementations, the pod may be manufactured as a single molding rather than having multiple parts that clip together. The pod may have its filter membrane bonded into this single substrate. The pod may have a filter with a smaller diameter such that a smaller population of cells may be treated. The pod may have markings molded into it to indicate fill level or have molded features to ensure orientation within the platform is consistent. The pod may have multiple features to enable it to be retained within a pod holder or stack outside of the apparatus. The pod may have a lid feature to facilitate incubation of cells within it. The pod may have a one-way check valve implemented to enable culture medium to be maintained within the cavity beneath the filter, or to support culture medium above the filter medium to keep cells in suspension post use of the pod.

As another example, some pods can include a hydroscopic foam located in the lower portion for pulling fluid from above the filter plate. Such an approach can be used to pull a delivery and/or payload solution off a cellular monolayer formed above the filter plate, thereby controlling a length of contact between the cell population and the delivery and/or payload solution. An example foam is 3M™ Tegaderm™ Foam Dressing (non-adhesive).

As another example, the lower portion does not include holes and can include a flat tissue cultured treated surface. Such an implementation can be suitable for adherent cell populations to enhance adherence. Such an implementation with a flat surface can be utilized for delivery to tissue explants or engineered tissues.

In some implementations, the pod can be suitable for culturing cells. Rather than immediately removing the cells from the pod, the cells can be cultured for a period of time, such as hours or days. In such implementations the pod can be formed of culture compatible materials and a pod lid can be provided.

In some implementations, the pod can include memory storing process parameters. For example, a pod memory can be programmed with the process parameters such that, when the pod is inserted into the cell engineering platform, a controller on the cell engineering platform reads, from the pod memory, the process parameters. The cell engineering can proceed using the process critical parameters, for example, via an automated fashion (e.g., an amount of solution delivered to the cells can be determined by the controller), or via displaying instructions to the user via a display. By having the process parameters stored on the pod prior to conducting the delivery process, repeatability can be improved because the user is not required to enter the process parameters into the platform.

In another example, the process parameters are first loaded into the controller of the cell engineering platform, and the delivery process is performed using those parameters. After completion of the process, the cell engineering platform can write to the pod memory the process parameters for future reference. These process parameters can include any parameter utilized or described herein as related to delivery of a payload into a cell. For example, the delivery protocol such as solution compositions, exposure lengths, incubation times, wash cycles, temperatures, spray characteristics, pressures, volumes (e.g., of delivery solution to be applied, media to introduce, and the like), cell characteristics, and the like.

In some implementations, the cell engineering platform can write information such as an experiment identifier, date, time, and the like, to the pod memory for future use and/or reference. In some implementations, pods can communicate with one another. For example, a container or housing adapted to hold multiple pods can include connections between the pods so that the container reads data from the memory of a first pod, and copies some or all of the data to the other pods contained in the container. Such an approach can also improve repeatability because, once the first pod is programmed with process critical parameters, that data is replicated to the other pods without modification to some or all of the data.

In some implementations, the pod can include a memory, a processor, and/or a communications module, such as a near-field or radio frequency identification (RFID) communication module capable of communicating with the cell engineering platform and/or other pods. In some implementations, the pod can include electrical contacts for communicating with the cell engineering platform when the pod is inserted into the cell engineering platform. Other implementations are possible.

### Example 1

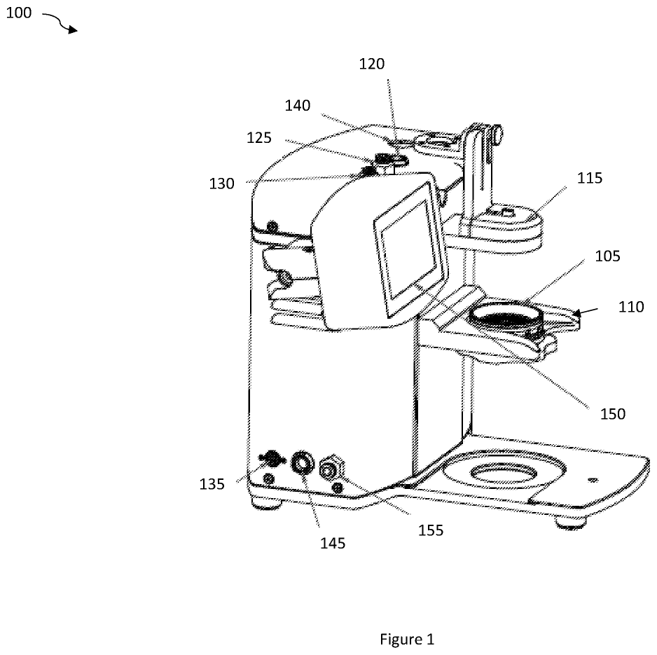

FIG. 1 is an isometric view of a computer aided design (CAD) drawing illustrating an example embodiment of a delivery platform 100 according to some embodiments disclosed herein. The delivery platform 100 includes a pod 105 configured to be received and positioned within a pod nest 110. An example pod 105 is illustrated in FIGS. 15-19. The pod 105 can include an upper portion 1605, a filter plate 1610, and a lower portion 1615. The pod 105 can provide a processing surface, via the filter plate 1610, on which cells can be provided for treatment and processing. For example, the filter plate 1610 can be configured to receive a filter for use in forming a monolayer of cells to be processed using the delivery platform 100.

The pod 105 can be received and positioned within the pod nest 110. In some embodiments, the atomizer nest 115 can be a fixed distance above the pod 105. The atomizer nest 115 can be a fixed distance from the pod nest 110 to reduce the number of variables or degrees of freedom available to the user thereby providing a system that is easier to use. For example, the atomizer nest 115 can be fixed about 75 mm above the pod 105. The pod nest 110 can include a circular opening to receive the pod 105. A lower portion 1615 of the pod 105 can be mated to the filter plate 1610 by coupling the lower portion 1615 with a portion of the filter plate 1610 extending through the circular opening of the pod nest 110. The pod nest 110 can provide support to the pod 105 and can maintain the position of the pod 105 during cell processing using the delivery platform 100. For example, the pod nest 110 can maintain the position of the pod 105 to ensure the treatment surface of the pod 105, e.g., the filter plate 1610, is sufficiently located to receive adequate amounts of delivery solution.

As further shown in FIG. 1, the delivery platform 100 includes an atomizer nest 115. The atomizer nest 115 can include an atomizer coupled to a delivery solution source configured within the delivery platform 100. The atomizer can atomize the delivery solution to provide the delivery solution to the pod 105 (e.g., in the form of a spray) to process or treat cells configured on the filter plate of the pod 105. The atomizer nest 115 can be coupled to the delivery solution source via a valve connector 120, such as a clippard value connector. The atomizer configured within the atomizer nest 115 can be configured to provide the delivery solution to the pod 105 at a predetermined pressure. The delivery platform 100 also includes a sample pressure connector 125 and an air pressure connector 130. The valve connector 120 serves to control delivery solution application to atomizer. The sample pressure connector 125 pressurizes the gas above the fluid in the Eppendorff reservoir to drive the sample into the atomizer. The gas pressure connector 130 supplies pressurized gas to the atomizer.

The delivery platform 100 also includes a power input 135. In some embodiments, the power input 135 can include a 2 channel direct current (DC) 24V power input 135. The power input 135 can be electrically coupled to the On/Off switch 140. The delivery platform 100 also includes a human machine interface (HMI) cable coupling 145. The HMI cable coupling 145 can be electrically coupled to the HMI 150. The HMI 150 can include a display, at least one data processor, and input devices configured to control operation of the delivery platform 100 and to perform the methods of cell treatment via delivery described herein. In some embodiments, the HMI 150 can include a touch screen interface. In some embodiments, the HMI 150 can include process guides, laboratory timers, and the like. The HMI cable coupling 145 can be configured to couple the HMI 150 to a computing device that is located separately from the delivery platform 100. In this way, data can be imported to or exported from the delivery platform 100.

The delivery platform 100 further includes an air supply coupling 155. The air supply coupling 155 can couple the delivery platform 100 to an air supply. The air supply can be used to provide air, via the air supply coupling 155, for use in configuring an amount of air to be provided with the delivery solution to the pod 105.

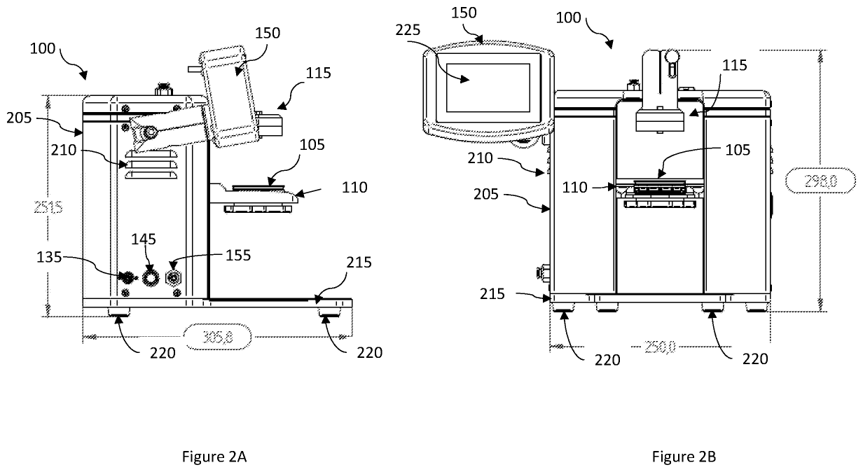

FIG. 2A is a side view of the delivery platform 100 shown in FIG. 1. As shown in FIG. 2A, the delivery platform 100 can include an enclosure 205. The enclosure 205 can include a number of cutouts corresponding to the power input 135, the HMI cable coupling 145, and the air supply coupling 155. Additional cutouts can be provided within the enclosure 205 without limitation. For example, the enclosure 210 can include a plurality of vents 210. The enclosure 205 can be affixed to a base plate 215. The base plate 215 can include a plurality of feet 220. In some embodiments, the feet 220 can be plastic and can include friction-reducing materials to secure the delivery platform 100 on a surface.

FIG. 2B is a front view of the delivery platform 100 shown in FIG. 1. As shown in FIG. 2B, the delivery platform 100 can include an HMI 150 and the HMI 150 can include a display 225. The display 225 can provide visualizations of data and user-interface controls corresponding to one or more aspects of operation of the delivery platform 100. For example, in some embodiments, the display 225 can provide touch screen controls configured to perform one or more operations of methods of delivery to cells. In some embodiments, the HMI 150 can include a timer and the timer, as well as timer controls, can be displayed via the display 225.

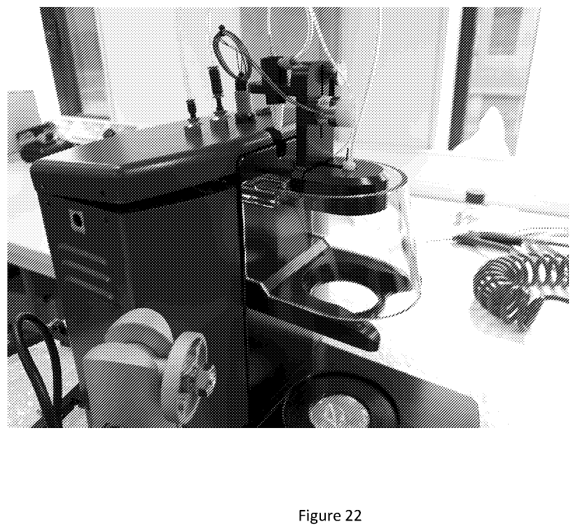

In some implementations, the delivery platform 100 can include a spray-guard device to contain atomization (e.g., overspray). In one example, the spray-guard is transparent, demi-cylindrical device that has the same internal diameter as the outer contour of the pod nest. In some implementations, the spray-guard is not a sealed device but affords some degree of containment. The spray-guard clips on to the front of the device. FIG. 22 illustrates an example spray-guard.

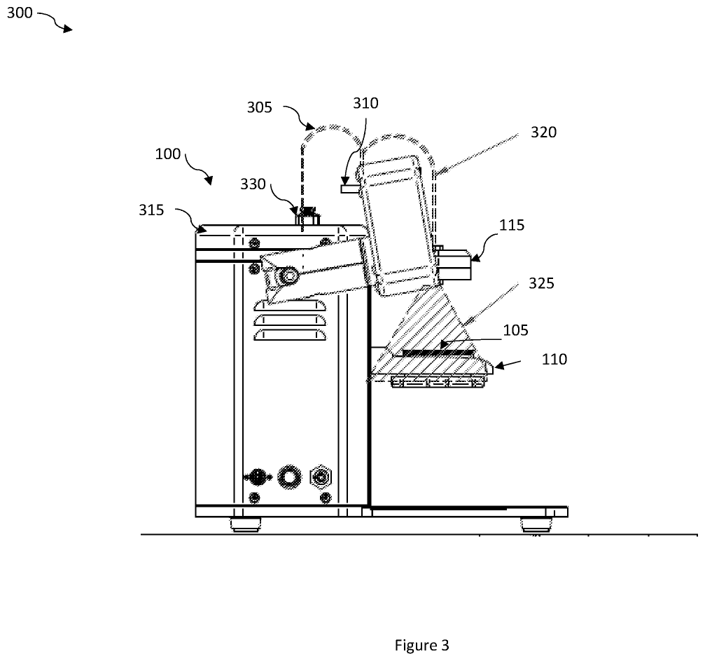

FIG. 3 is a diagram 300 illustrating a side view of another example embodiment of the delivery platform 100 shown in FIG. 1, according to some embodiments disclosed herein. As shown in FIG. 3, the valve can be coupled to the atomizer nest 115 via one or more portions of tubing. A pneumatic fitting 330 can include, for example, a Festo 6 mm to 6 mm bulkhead fitting (Catalogue No. 193951). For example, a first portion of tubing 305 can couple the valve to an Eppendorf base support 310. The Eppendorf base support 310 can be coupled to a top cover 315 of the delivery platform 100.

The Eppendorf base support 310 can include a bracket that holds the payload reservoir in space. An example reservoir includes a 1.5 mL Eppendorf brand centrifuge vial. The reservoir may or may not be permanently fixed in place as the mechanism for securing it to the Eppendorf base support 310.

A second portion of tubing 320 can coupled the Eppendorf base support 310 to the atomizer nest 115. A delivery solution can be conveyed from a source within the delivery platform 100, through the valve and to the Eppendorf base support 310 via the tubing 310. The delivery solution can be further provided to the atomizer nest 115 via tubing 320. Once received within the atomizer nest 115, the delivery solution can be provided to the pod 105 positioned within the pod nest 110. The atomizer configured within the pod nest 115 can be configured to deliver the delivery solution to the pod 105 with a spray pattern 325. The spray pattern 325 can be configurable based on a pressure setting at which the delivery solution is provided. In some embodiments, the spray pattern 325 can be associated with a configuration of an atomizer within the atomizer nest 115. Dimensions of the spray pattern 325, such as a spray angle, a coverage area, and/or a center point can be configurable aspects of the atomizer nest 115.

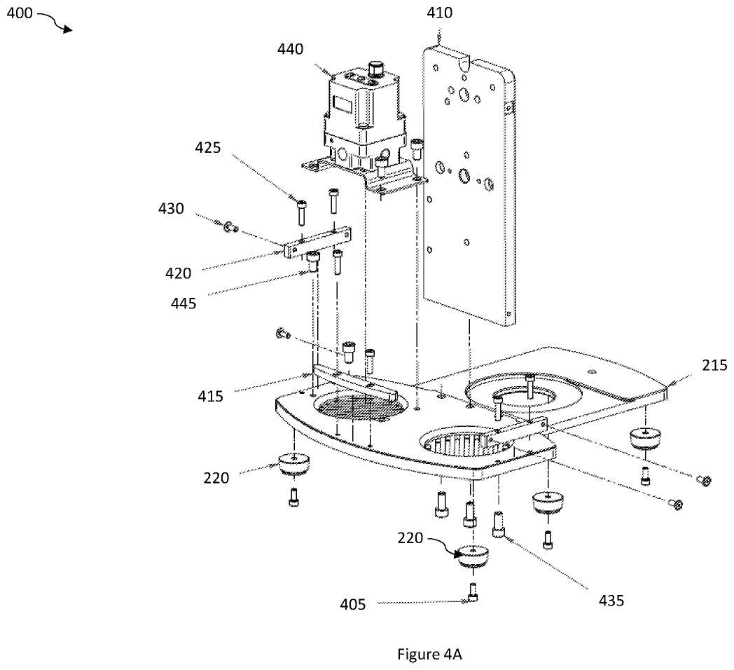

FIG. 4A is an isometric view of a CAD drawing illustrating an example embodiment of a base assembly 400 of the delivery platform 100 shown in FIG. 1. As shown in FIG. 4A, the base assembly 400 includes the base plate 215 and feet 220. Each foot 220 can be secured to the base plate 215 via a screw 405. In some embodiments, the screw 405 can include a M4×10 stainless steel screw. As further shown in FIG. 4A, the base assembly 400 includes an upright mounting spine 410. The upright mounting spine 410 can provide a base of support and a coupling mechanism for the pod nest 110, and the atomizer nest 115. The upright mounting spine 410 can be coupled to the base 215 and to the enclosure 205. The enclosure 215 can be coupled to the base assembly 400 via one or more supports. For example, the base assembly 400 includes a first rear cover support 415 and a second rear cover support 420. The second rear cover support 420 can be coupled to the base plate 215 via one or more screws 425. In some embodiments, the screws 425 can be M4×16 stainless steel screws. The enclosure 215 can be coupled to the second rear cover support 420 via one or more screws 430. In some embodiments, the screws 430 can include M4×10 ultra low head screws. The upright mounting spine can be coupled to the base plate 215 via one or more screws 435. In some embodiments, the screws 435 can include M6×16 stainless steel screws.

As further shown in FIG. 4A, the base assembly 400 includes a pressure regulator 440. The pressure regulator 440 can be secured to the base plate 215 via one or more screws 445. The pressure regulator 440 can be coupled to the power input 135 via a circuit board. The pressure regulator 440 can be configured to control an amount of pressure of the delivery solution provided to the pod 105 via the atomizer nest 115.

The pressure regulator 440 is coupled to the fluid sources via a network of pneumatic connections, as illustrated in FIG. 4B, which includes a pneumatic diagram of some implementations of the delivery platform 100. The regulator 440 has a maximum input pressure range of 1 MPa and an output range of 0.005 to 0.5 MPa and a maximum flow rate of 200 LPM.

In some embodiments, the screws 445 can include M6×10 socket head cap screws.

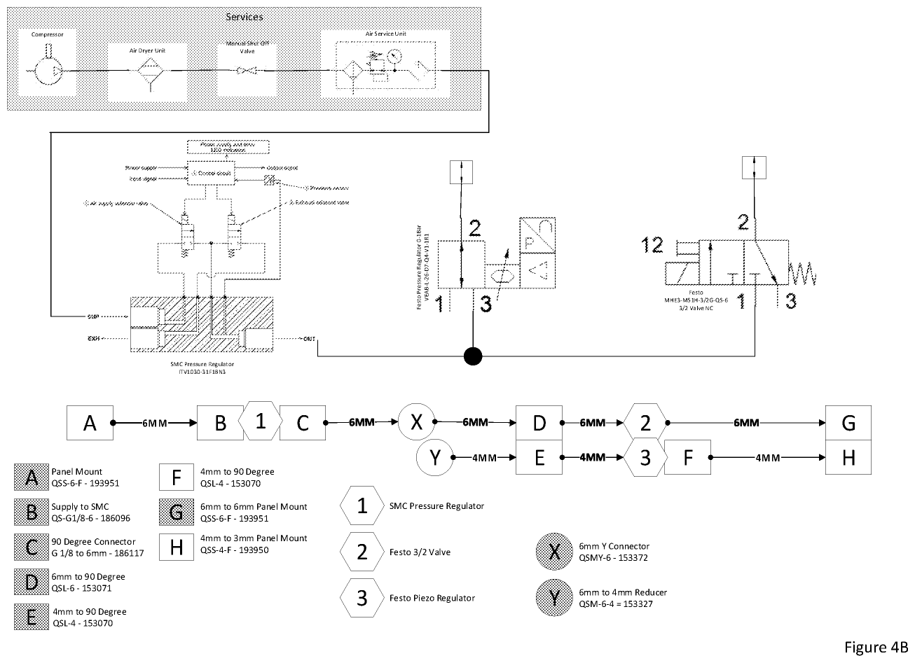

FIG. 5 is an isometric view of a CAD drawing illustrating an example embodiment of a spine assembly 500 of the delivery platform 100 shown in FIG. 1. As shown in FIG. 5, the atomizer nest 115 can be coupled to the upright mounting spine 410. The atomizer nest 115, shown within the dash-line box, includes a spray head base mounting platform 505 and a clippard module upper mount 510. A plurality of dowel pins 515 couple the clippard module upper mount 510 to the spray head base mounting platform 505. In some embodiments, the dowel pins 515 can be 4×20 mm. The clippard module upper mount 510 can further be coupled to the spray head base mounting platform 505 via a screw 520. In some embodiments, the screw can be an M6×16 socket head cap screw. The clippard module upper mount 510 can couple to the Eppendorf base support 310 via a knob 525. In some embodiments, the knob 525 can include a knurled thumb knob 525. The knob 525 can include a screw, such as a M4×10 mm screw for coupling the clippard module upper mount 510 to the Eppendorf base support 310.

As further shown in FIG. 5, the atomizer nest 115 also includes a clippard module lower mount 530. The clippard module lower mount 530 can be coupled to the spray head base mounting platform 505 via a plurality of magnets 535. In some embodiments, the magnets 530 can be 6×6 mm. The clippard module lower mount 535 can be further secured to the spray head base mounting platform 505 via a plurality of screws 540. In some embodiments, the screws 540 can include M3×6 mm flat head cap screws.

The spray head base mounting platform 505 can be coupled to the upright mounting spine 410 via a plurality of dowel pins 545. In some embodiments, the dowel pins 545 can be 6×25 mm. A screw 550 further couples the spray head base mounting platform 505 to the upright mounting spine 410. In some embodiments, the screw 550 can include a M6×20 stainless steel screw. The spine assembly 500 also includes a shaft 555. The shaft 555 can be configured for mounting the electrical and pneumatic subcomponent base plate. In some embodiments, the shaft 555 can include a rotary stepped shaft 555.

As further shown in FIG. 5, the pod nest 110 can be coupled to the upright mounting spine 410 via a plurality of bushings 560. In some embodiments, the bushings 560 can include notched-type bushings. The pod nest 120 can be configured to slide down onto the bushings 560. The pod nest 110 can be also be coupled to the upright mounting spine 410 via a screw 565. In some embodiments, the screw 565 can include a M6×10 socket head cap screw.

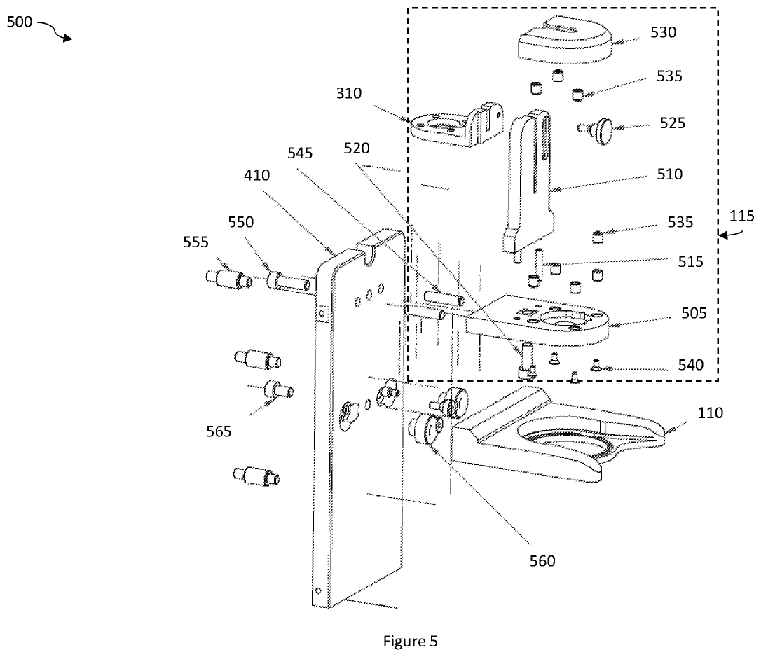

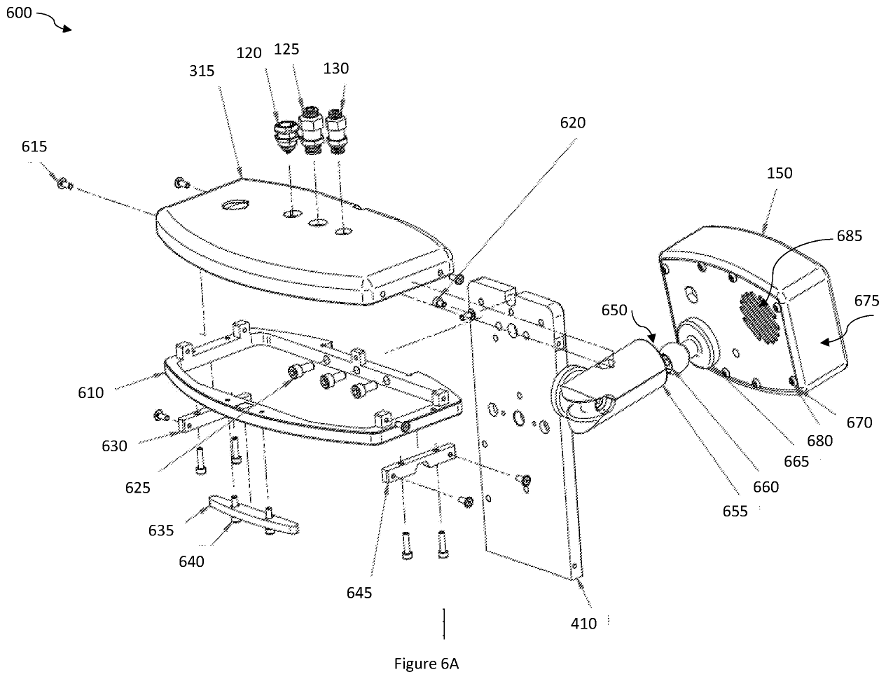

FIG. 6A is an isometric view of a CAD drawing illustrating an example embodiment of a top assembly 600 of the delivery platform 100 shown in FIG. 1. The top assembly 600 includes a top cover 315. The top cover 315 can be secured to a support rib 610 via a plurality of screws 615, In some embodiments, the screws 615 can include M4×10 ultra low head screws. The top cover 315 can also include cutouts for the clippard valve connector 120, the sample pressure connector 125, and the air pressure connector 130. In some embodiments, the clippard valve connector 120 can include a 2 pin socket connector configured with a blue nut. In some embodiments, the sample pressure connector 125 can include a bulkhead tube fitting. In some embodiments, the air pressure connector 130 can include a push-in bulkhead connector. The top assembly 600 also includes a screw 620 configured to secure a folded section of the outer cover to the central spine 410, which is illustrated in FIG. 6B. In some embodiments, the screw 620 can include a M4×6 stainless steel screw.

As shown in FIG. 6A, the upright mounting spine 410 can be secured to the support rib 610 via a plurality of screws 625. In some embodiments, the screws 625 can be M6×16 stainless steel screws. Additionally, the top assembly 600 can include one or more supports. Support 630 can be coupled to the support rib 610 via a plurality of screws. Support 635 can be coupled to the support rib 610 via a plurality of screws 640. In some embodiments, the screws 640 can be M4×16 stainless steel screws. Support 645 can also be coupled to the support rib 610 via a plurality of screws.

As further shown in FIG. 6A, the HMI 150 can be affixed to a ball end joint assembly 650. The ball joint assembly 650 can allow the HMI 150 to be positioned in a manner suitable for viewing by an operator of the delivery platform 100. The ball end joint assembly 650 can be coupled to portions of the enclosure 205 previously described in relation to FIG. 2. The ball end joint assembly 650 can include a ball joint socket 655. The ball joint socket 655 can be coupled to a ball end joint 660. In some embodiments, the ball end join 660 can include a M8×40 stainless steel screw. The ball end joint assembly 650 also includes a joint assembly mounting plate 665, which can be coupled to the HMI mounting plate 670. The HMI mounting plate 670 can be secured to a HMI front enclosure 675 via a plurality of screws 680. In some embodiments, the screws 680 can include M4×10 button stainless steel screws. As further shown in FIG. 6A, the HMI mounting plate 670 can include a plurality of cutouts 685 to release heat generated by the display 155 and/or the circuitry of the HMI 150.

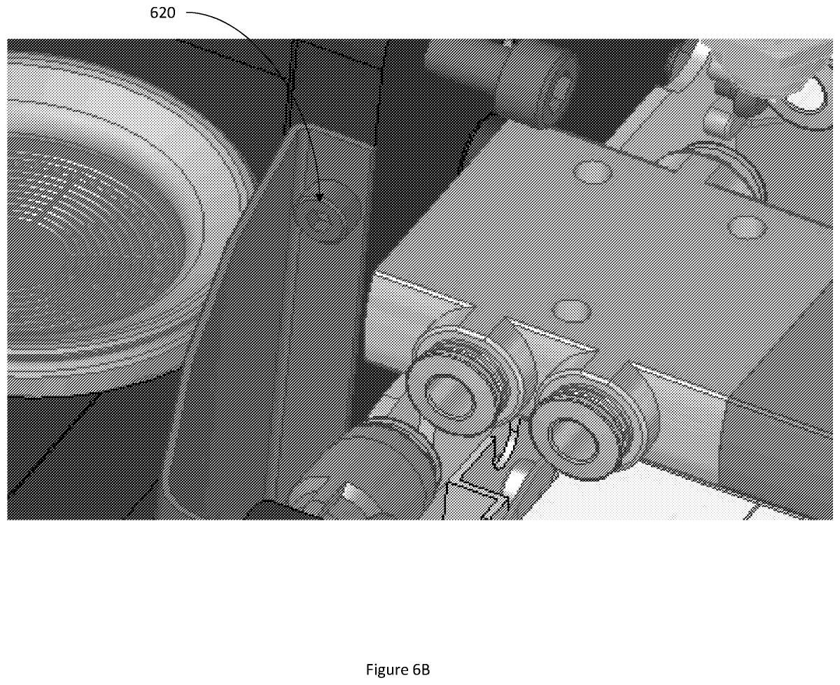

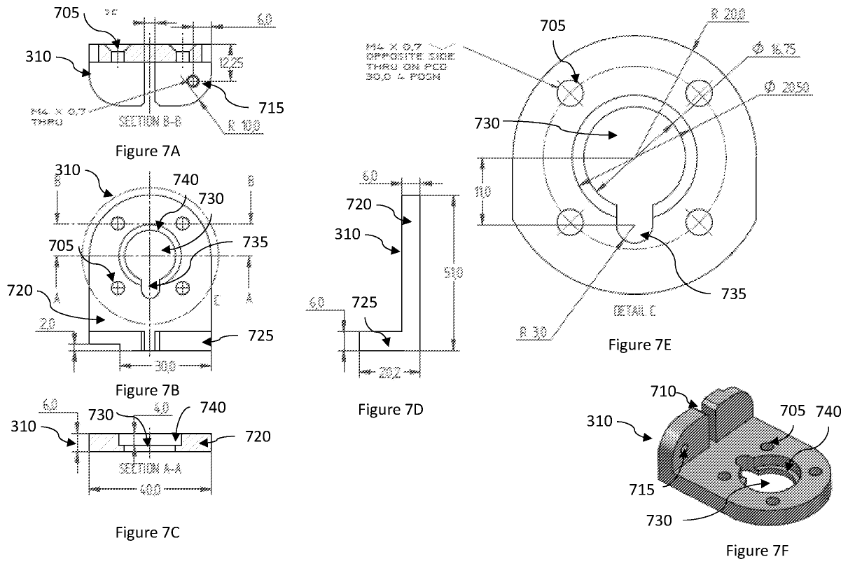

FIGS. 7A-7E are CAD drawings illustrating an example Eppendorf base support of the delivery platform 100 of FIG. 1. The Eppendorf base support shown in FIGS. 7A-7E corresponds to the Eppendorf base support 310 shown in FIGS. 3 and 5. The dimensions of the Eppendorf base 310 shown in FIGS. 7A-7E are exemplary and not intended to limit the size or configuration of the Eppendorf base support 310. The Eppendor base support 310 includes a bracket that holds the payload reservoir in space. The payload reservoir is not secured in place and a user is free to remove it from the bracket without disengaging any clamping mechanism.

FIG. 7A shows a horizontal cross-sectional view of a first end of the Eppendorf base 310. As seen in FIG. 7A, the Eppendorf base 310 includes a plurality of holes 705 and a slot 710. The holes 705 are features for employing a clamping mechanism. The slot 710 facilitates the screws that secure the Clippard Pinch Valve to the bracket 510 as part of the assembly 115 and allows the distance between the pinch valve and the atomiser to be varied.

As further shown in FIG. 7A, the Eppendorf base 310 includes a hole 715 configured to receive the screw portion of knob 525 shown and described in relation to FIG. 5.

FIG. 7B shows a top-down view of the Eppendorf base 310. The Eppendorf base 310 includes a mounting surface 720 and a flange portion 725 extending from the mounting surface 720. The mounting surface 720 can include a plurality of holes 705 configured to mount the Eppendorf base 310 to the top cover 315. The mounting surface 720 includes an opening 730 configured with a notch 735 at a location of the opening 730 closest to the flange 725. The opening 730 can include a recessed portion 740 extending circumferentially around a portion of the opening 730. The payload reservoir sits in opening 730. Notch 735 is a positioning feature for an Elveflow subcomponent (not shown) which facilitates the sealing of the payload reservoir and transfer of fluid from the reservoir to the atomiser.

FIG. 7C shows a side-view of the mounting surface 720. The recessed portion 740 can be formed in an upper surface of the mounting surface 720 and the circular opening 730 can extend through the mounting surface 720.

FIG. 7D shows a side-view of the Eppendorf base 310. The Eppendorf base 310 can include the mounting surface 720 arranged orthogonally to the flange 725.

FIG. 7E shows a top-down view of the detail area “C” shown in FIG. 7B. As shown in FIG. 7E, the detail area “C” illustrates a plurality of holes 705 arranged around the circular opening 730. Although the holes 705 are arranged in a square shaped formation around the circular opening 730, the holes 705 can be arranged in any variety of configurations around the circular opening 730 without limitation. The notch 735 can be configured to extend through the mounting surface 720.

FIG. 7F is a drawing illustrating an isometric view of the Eppendorf base 310.

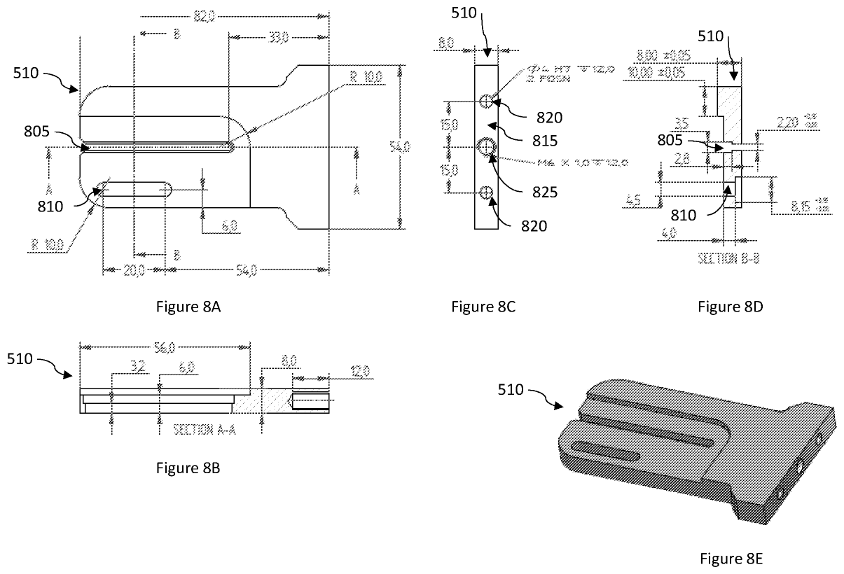

FIGS. 8A-8E are CAD drawings illustrating an example of a clippard module upper mount 510 of the delivery platform 100 of FIG. 1. The clippard module upper mount 510 shown in FIGS. 8A-8E corresponds to the clippard module upper mount 510 shown in FIG. 5. The dimensions of the clippard module upper mount 510 shown in FIGS. 8A-8E are exemplary and not intended to limit the size or configuration of the clippard module upper mount 510.

As shown in FIG. 8A, the clippard module upper mount 510 can include a slot 805. Slot 805 can facilitate mounting position of the Clippard Pinch Valve relative to the atomiser i.e. shorter or longer tube length between pinch valve and the atomiser.

The clippard module upper mount 510 can also include a slot 810, which can be closed at either end. The knob 525, shown in FIG. 5, can be configured to extend through the slot 810 to couple the clippard module upper mount 510 with the Eppendorf base 310.

FIG. 8B shows a side view of the clippard module upper mount 510. The clippard module upper mount 510 can include one or more recessed surfaces configured therein. FIG. 8C is an end view of a mounting surface 815 of the clippard module upper mount 510. The mounting surface 815 can couple to the spray head base mounting platform 505 via one or more dowel pins 515 as shown in FIG. 5. The dowel pins 515 can be received within holes 820 as shown in FIG. 8B. Hole 825 can be a threaded hole configured to receive the screw 520 shown in FIG. 5.

FIG. 8D is a vertical cross-sectional view of the clippard module upper mount 510 showing the slots 805 and 810, as well as the recessed surfaces configured on the clippard module upper mount 510.

FIG. 8E is a drawing illustrating an isometric view of the clippard module upper mount 510.

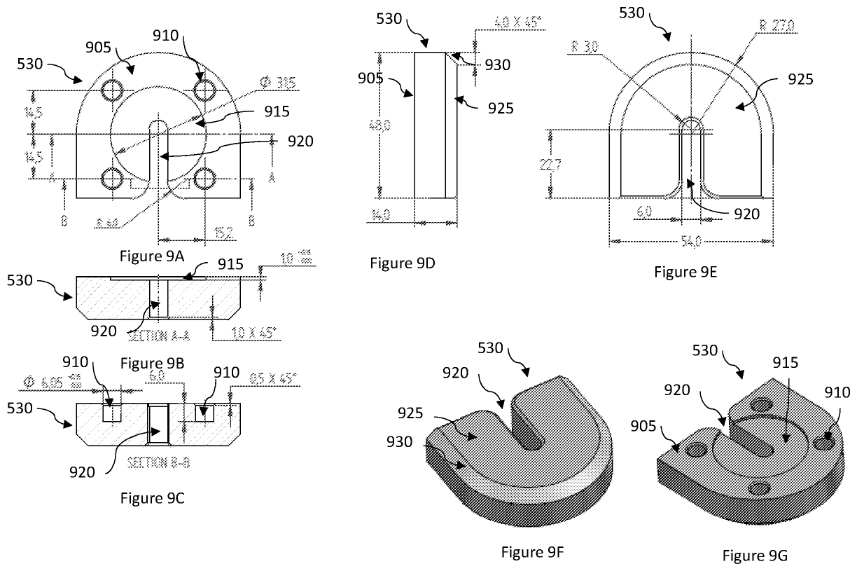

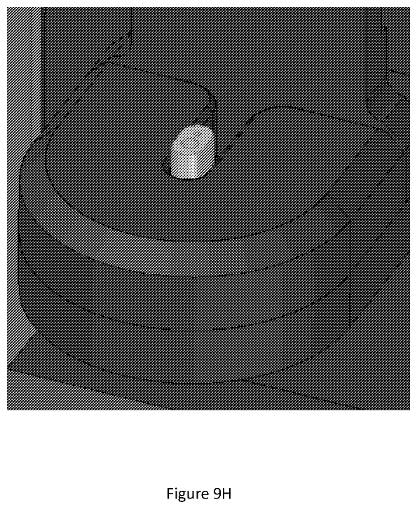

FIGS. 9A-9G are CAD drawings illustrating an example clippard module lower mount 530 of the delivery platform 100 of FIG. 1. The clippard module lower mount 530 shown in FIGS. 9A-9G corresponds to the clippard module lower mount 530 shown in FIG. 5. The dimensions of the clippard module lower mount 530 shown in FIGS. 9A-9G are exemplary and not intended to limit the size or configuration of the clippard module lower mount 530.

As shown in FIG. 9A, shows a lower surface 905 of the clippard module lower mount 530. The lower surface 905 can include a plurality of holes 910. The holes 910 can be configured to receive the magnets 535 shown in FIG. 5. The clippard module lower mount 530 can be coupled to the spray head base mounting platform 505 via the magnets 535 positioned within the holes 910. As shown in FIG. 9A, the plurality of holes 910 can be arranged around a circular recess 915 formed within the lower surface 905. Although the holes 910 are arranged in a square shaped formation around the circular recess 915, the holes 905 can be arranged in any variety of configurations around the circular recess 915 without limitation. The slot 920 can extend through the clippard module lower mount 530. Slot 920 facilitates protrusion of the back end of the atomiser through its clamping mount, as illustrated in FIG. 9H.

FIG. 9B shows a cross-sectional view of the clippard module lower mount 530 from the perspective of lines A-A shown in FIG. 9A. FIG. 9C shows a cross-sectional view of the clippard module lower mount 530 from the perspective of lines B-B shown in FIG. 9A. FIG. 9C shows a side view of the clippard module lower mount 530 showing the lower surface 905 and the upper surface 925. The upper surface 925 can include a beveled edge 930.

FIG. 9E shows a top view of the clippard module lower mount 530. The slot 920 can be sized to extend about half way through the clippard module lower mount 530. The beveled edge 930 can extend fully about the circumference of the upper surface 925. FIG. 9F is a drawing illustrating an isometric view of the upper surface 925 of the clippard module lower mount 530. FIG. 9G is a drawing illustrating an isometric view of the lower surface 905 of the clippard module lower mount 530.

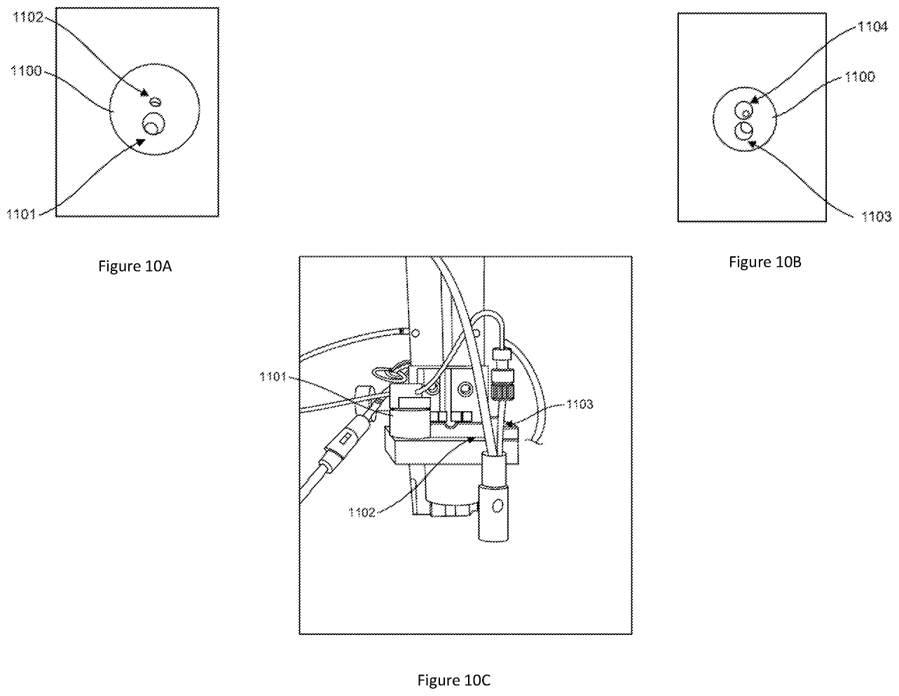

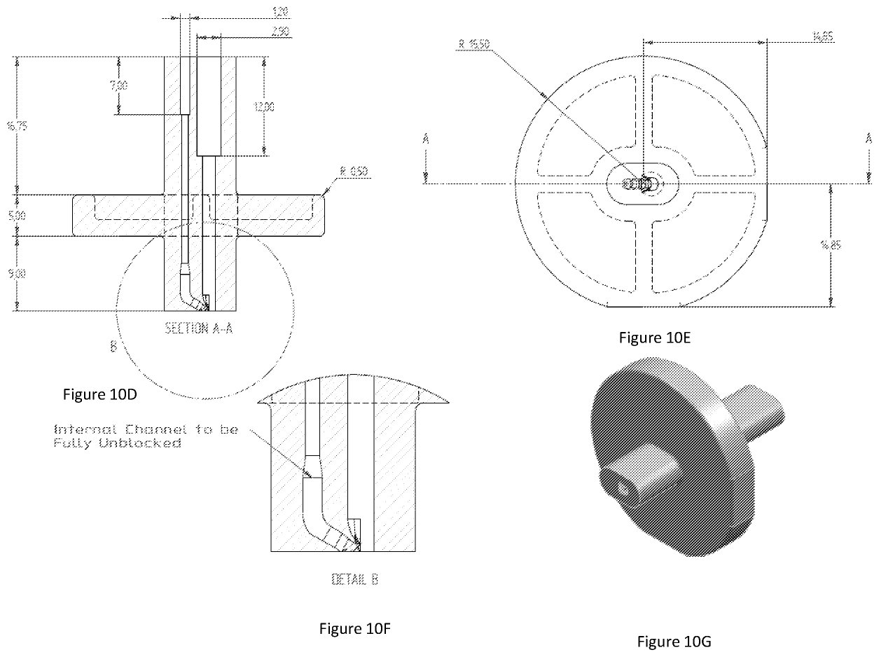

FIG. 10A-10C show an exemplary embodiment of the atomizer 1100 for the spraying process. Referring to FIGS. 10A-10C, the atomizer 1100 includes a liquid orifice 1101 and a gas orifice 1102 on a lower surface thereof (FIG. 10A). On an upper surface of the atomizer 1100, a liquid tubing inlet 1103 and an air tubing inlet 1104 may be formed (FIG. 10B). Accordingly, the liquid orifice 1101 is connected to a liquid reservoir through the liquid tubing inlet 1103, and the gas orifice 1102 is connected to a gas reservoir through the air tubing inlet 1104 as shown in FIG. 10C. The gas reservoir may be an air cylinder or an air pump, and may be provided with a valve.

In some implementations, an LB-100 nebulizer can be utilized. In some implementations, the values at which the nebulizer is used involves the atomization of a volume between about 10⁻³⁰⁰ μl of cell delivery solution. Exemplary nebulizers are described in U.S. Pat. No. 5,411,208 or U.S. Pat. No. 6,634,572, hereby incorporated by reference in their entireties. Additional nebulizers are commercially available, e.g., from DuraMist™ Nebulizer (Sigma-Aldrich GXARG1DM04-1EA), Nebulizer, OneNeb, series 2 inert concentric type nebulizer, or use with ICP-OES (Agilent Technologies G8010⁻⁶⁰²⁹³). In embodiments, the nebulizer can be an ultrasonic nebulizer, or a vibrating mesh nebulizer. Input and output tubes can be welded or Hospira Spinning Spires closed connectors can be utilized. FIG. JOD-G illustrate another example atomizer. Other atomizer designs and geometries are possible.

In some implementations, an atomizer adaptor can be included, which can adjust an orientation of the atomizer. For example, some atomizers can spray in a direction 1-5 degrees off their main axis. An adaptor can be included that holds the atomizer in a manner to adjust the orientation, for example, so the atomizer directs atomized solution in a direction perpendicular to the face of the pod 105 filter plate 1610.

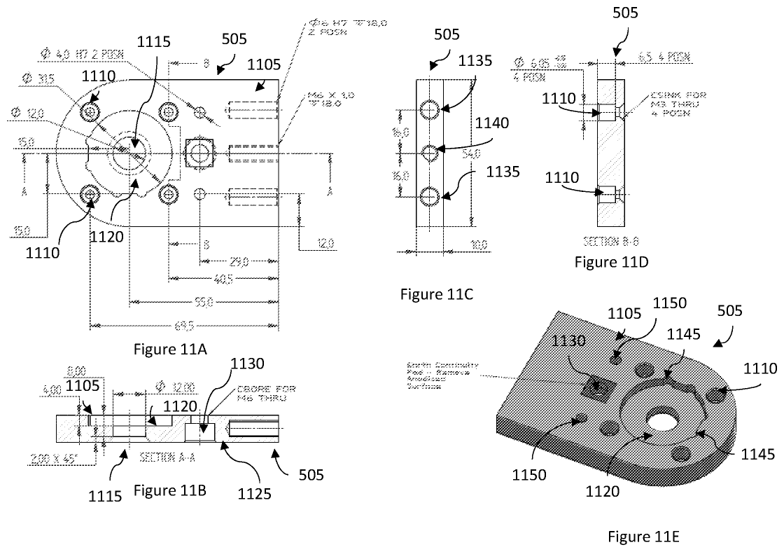

FIGS. 11A-11E are CAD drawings illustrating an example spray head base mounting platform 505 of the delivery platform 100 of FIG. 1. The spray head base mounting platform 505 shown in FIGS. 11A-11E corresponds to the spray head base mounting platform 505 shown in FIG. 5. The dimensions of the spray head base mounting platform 505 shown in FIGS. 11A-11E are exemplary and not intended to limit the size or configuration of the spray head base mounting platform 505.

FIG. 11A is a top view of an upper surface 1105 of the spray head base mounting platform 505. As shown in FIG. 11A, the spray head base mounting platform 505 includes a plurality of holes 1110 configured with respect to a circular opening 1115 and a recessed surface 1120. As shown in FIG. 11A, the plurality of holes 1110 can be arranged around the circular opening 1115 and the recessed surface 1120. Although the holes 1110 are arranged in a square shaped formation around the circular opening 1115, the holes 1110 can be arranged in any variety of configurations around the circular opening 1115 and/or the recessed surface 1120 without limitation. The holes 1110 can receive the screws 540, shown in FIG. 5, for use in coupling the clippard module lower mount 530 to the spray head base mounting platform 505.

FIG. 11B shows a horizontal cross-sectional view of the spray head base mounting platform 505 from the perspective of lines A-A shown in FIG. 11A. As shown in FIG. 11B, the circular opening 1115 can include a flanged portion at the lower surface 1125. The spray head base mounting platform 505 also includes a hole 1130 therethrough. The hole 1130 can be configured to receive screw 520, shown in FIG. 5, to aid in securing the clippard module upper mount 510 to the spray head base mounting platform 505.

FIG. 11C shows an end view of the spray head base mounting platform 505. As shown in FIG. 11C, holes 1135 can be provided to receive dowel pins 545 shown in FIG. 5. Hole 1140 can be configured to receive screw 550 to couple the spray head base mounting platform 505 to the upright mounting spine 410.

FIG. 11D shows a cross-sectional view of the spray head base mounting platform 505 from the perspective of lines B-B shown in FIG. 11A. The holes 1105 can include a counter sink portion to receive the screws 540.

FIG. 11E is an isometric view of the spray head base mounting platform 505. As shown in FIG. 11E, a plurality of notches 1145 can be formed in the walls surrounding the recessed surface 1120. The notches 1145 are features to fix the radial orientation of a plurality of test atomizers. Holes 1150 can be configured to receive dowel pins 515 to couple the spray head base mounting platform 505 to the upright mounting spine 410 as shown in FIG. 5.

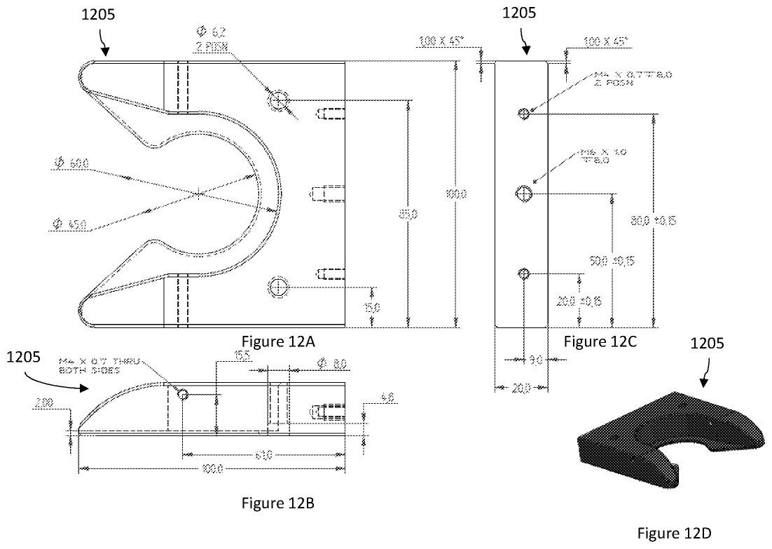

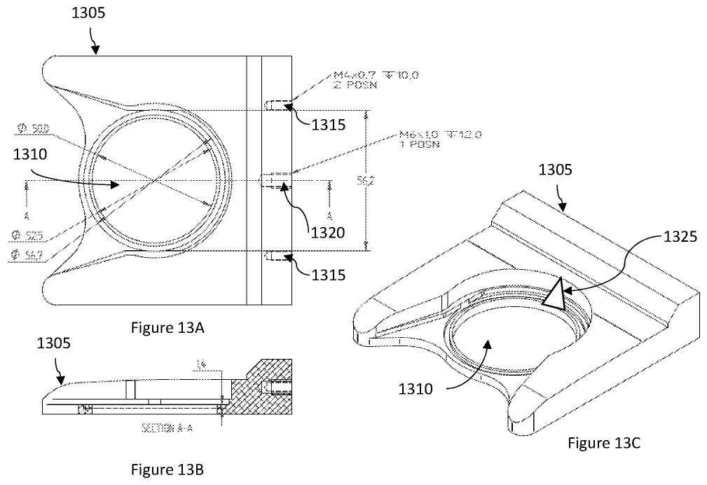

FIGS. 12A-12D are CAD drawings illustrating an example pod nest 1205 of an exemplary embodiment of the delivery platform 100 of FIG. 1. The dimensions of the pod nest 1205 shown in FIGS. 12A-12D are exemplary and not intended to limit the size or configuration of the pod nest 1205.

FIGS. 13A-13C are CAD drawings illustrating another example pod nest 1305 of an exemplary embodiment of the delivery platform 100 of FIG. 1. The dimensions of the pod nest 1305 shown in FIGS. 13A-13C are exemplary and not intended to limit the size or configuration of the pod nest 1305.

FIG. 13A shows a top view of the pod nest 1305. The pod nest 1305 includes a circular pod receiving area 1310. A pod 105 can be received within the pod receiving area 1310. The pod nest 1305 can include a plurality of holes 1315 configured to couple with the bushings 560 shown in FIG. 5. The pod nest 1305 also includes a hole 1320 configured to receive the screw 565 shown in FIG. 5. The pod nest 1305 can be secured to the upright mounting spine 410 via the bushings 560 and the screw 565.

FIG. 13B shows a cross-sectional side view of the lower portion 1305 of the pod nest 110 from the perspective of lines A-A shown in FIG. 13A.

FIG. 13C shows an isometric view of the pod nest 1305 including the circular pod receiving area 1310. In some embodiments, the pod nest 1305 can include a sensor 1325. The sensor 1325 can include a camera, a radio frequency (RF) identification (ID) scanner, or an IR sensor. The sensor 1325 can be configured to determine an event, such as sufficient drainage of delivery solution from the pod 105. In this way, event-driven workflows associated with intracellular delivery can be achieved using the delivery platform 100. In some implementations, the sensors may be included in the pod 105, such as in upper portion 1605, filter plate 1610, and/or lower portion 1615. In such implementations, electrical connections can be included in the pod 105 and the pod nest 110 for connecting to the sensors, for example, to provide power and/or make sensor measurements.

In some embodiments, the pod nest 110, 1205, 1305 can be configured to vibrate to aid settling cells into a monolayer within the pod or to aid recovery of cells from within the pod. Vibrational functionality may be added directly to the pod nest 110 via vibrational elements added onto or into the pod nest. Examples of vibrational elements include eccentric motors or liner resonant displacement (LRD) devices. With regard to the mechanical resonance character of the pod nest (connected to the device) vibrational perturbation may be added during process steps. Vibrational perturbations in the frequency range 50 Hz to 2500 Hz and physical excursions (e.g., amplitude) of 2 mm may provide appropriate mixing or agitation. The pod nest 110 may be mechanically coupled with rubber or elastomeric mounts to facilitate agitation. Agitation may be independently applied via a signal generator in the X, Y or Z plane using LRD devices.

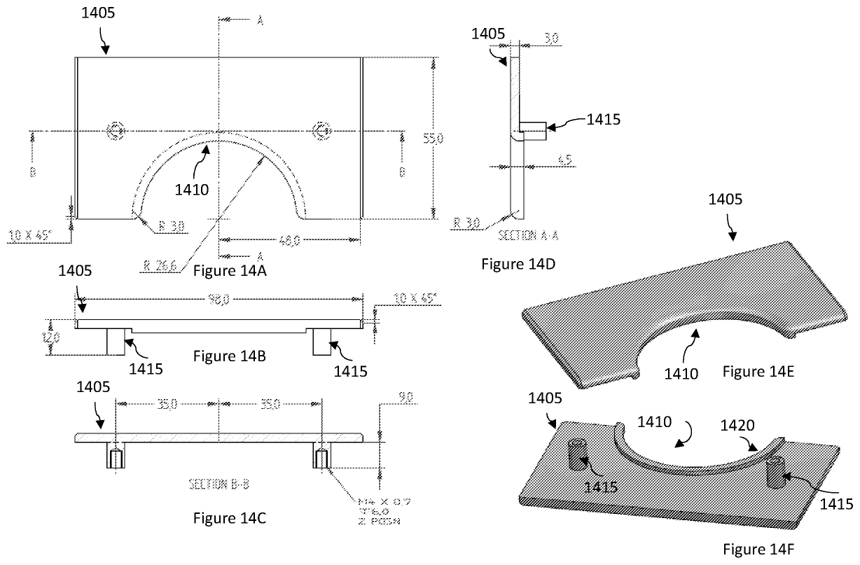

FIGS. 14A-14F are CAD drawings illustrating an example pod nest cover 1405 of the delivery platform 100 of FIG. 1. The dimensions of the pod nest cover 1405 shown in FIGS. 14A-14F are exemplary and not intended to limit the size or configuration of the pod nest cover 1405. The example pod nest cover 1405 is configured to engage with pod nest 1205 by slotting element 1415 into mating holes within pod nest 1205.

FIG. 14A shows a top view of the pod nest cover 1405. The pod nest cover 1405 includes a semi-circular cutout 1410 into which a pod 105 can be received with placed within the pod nest 110.

FIG. 14B shows a side view of the pod nest cover 1405. The pod nest cover 1405 can include a plurality of extensions 1415 protruding from the bottom of the pod nest cover 1405.

FIG. 14C shows a horizontal cross-sectional view of the pod cover 1405 from the perspective of lines B-B shown in FIG. 14A. The extensions 1415 can include tapped M4 for the purposes of rigidly fixing pod nest cover 1405 to pod nest 1205. This can be achieved by mating 2 screws from the underside of pod nest 1205 with counter bored holes on pod nest 1205.

FIG. 14D shows a vertical cross-sectional view of the pod cover 1405 from the perspective of lines A-A shown in FIG. 14A.

FIG. 14E shows an isometric view of a top surface of the pod cover 1405. FIG. 14F shows an isometric view of a bottom surface of the pod cover 1405. As shown in FIG. 14F, the bottom surface of the pod cover 1405 incudes the extensions 1415 protruding from the bottom surface. The bottom surface of the pod cover 1405 also includes a flange 1420 extending circumferentially around the semi-circular cutout 1410 and away from the bottom surface of the pod cover 1405 to cover the rim of the pod 105 to prevent the pod 105 from lifting up out of the pod nest 1205.

FIG. 15 is an image of an example embodiments of a pod assembly 1500 for use in the delivery platform 100 shown in FIG. 1. The pod assembly 1500 can include a plurality of mate-able components, which can be coupled and uncoupled while performing delivery to cells using the delivery platform 100. In some embodiments, portions of the pod assembly 1500 can be configured for use with the delivery platform 100. In some embodiments, portions of the pod assembly 1500 can be configured for repeated use with the delivery platform 100.

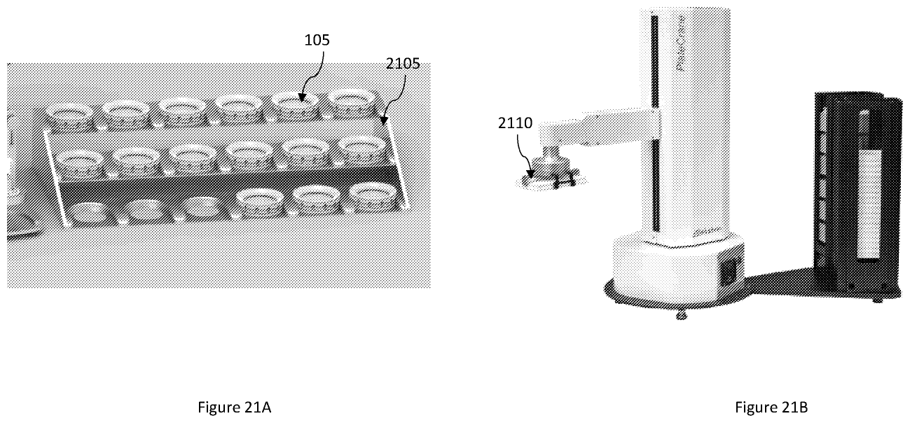

In some implementations, pods 105 maybe stacked temporarily on a frame adjacent or connected to the device. The frame can organize and retain a small number of pods, for example 6 pods or 12 pods 105 ready for insertion into the machine manually or automatically. Pods 105 retained in the frame may be pre-treated or preloaded with cells whilst retained within the frame. The pods 105 can be in the frame for a limited time before and after the experiment or device usage. In some implementations, when an experimental run is in progress, the operator may manually transfer pods from the frame into the pot nest, transfected the cells within the part and move them to a second frame for retaining post transfected pods. This process can also be enabled automatically. The frame may be of open construction to aid cleanability. The frame may be manual, for example as shown by frame 2105 illustrated in FIG. 21A or the frame may be configured for use with a plate stacker, for example, as shown by frame 2110 illustrated in FIG. 21B. Commercially available plate stackers are available from Hudson Robotics, Inc. of Springfield Township, New Jersey, USA. In some implementations, the frame can include sensors and/or communications for communicating with a pod. The frame can include position sensors and/or timers.

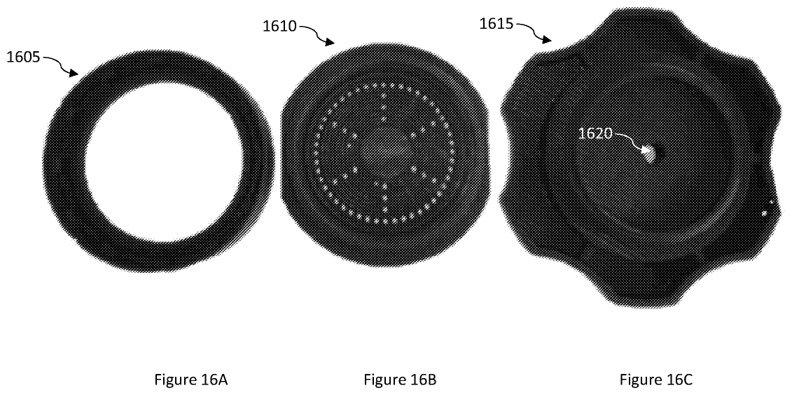

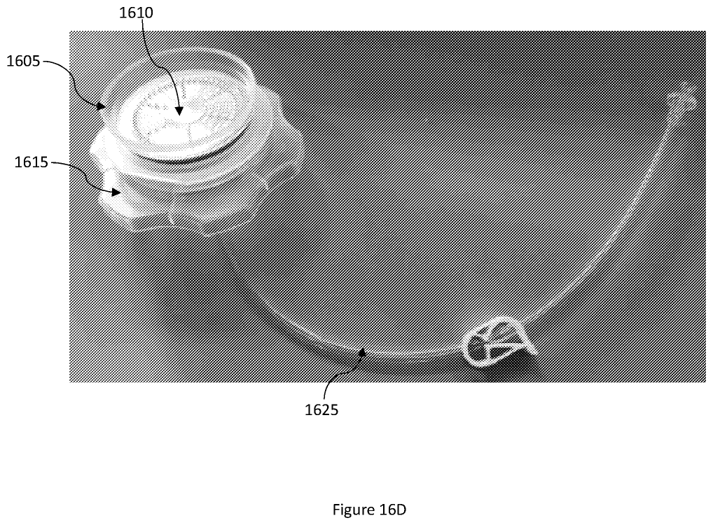

FIGS. 16A-16C are images of example embodiments of components of the pod assembly 1500 shown in FIG. 15. FIG. 16A shows a retainer ring 1605 of the pod assembly 1500. FIG. 16B shows a filter plate 1610 of the pod assembly 1500. FIG. 16C shows a filter plate coupling 1615 of the pod assembly 1500. During use within the delivery platform 100, a pod 105 can be configured to include retainer ring 1605 and the filter plate 1610. During some aspects of use, such as draining the delivery solution or the like, the filter plate coupling 1615 can be coupled to the pod 105 via the filter plate 1610 to provide a drainage path from the pod 105.

As shown in FIG. 16A, the retainer ring 1605 is a ring-shaped component configured to maintain a suitable fluid level within the pod 105. In some implementations, such as where the pod is configured as a disposable pod intended for single-use, the retainer ring 1605 can be pre-formed and integrated with the remainder of the pod. The retainer ring 1605 material can be similar to that of the other aspects of the pod substrate.

As shown in FIG. 16B, the filter plate 1610 can include a plurality of holes to allow fluid to drain therethrough. In some embodiments, the filter plate 1610 can receive a filter upon which cells can be provided for delivery of a payload. The plurality of holes or apertures can be formed in a variety of non-limiting patterns suitable to provide sufficient retention of cells and draining of solutions. For example, the apertures may be aligned around an outer diameter of the filter plate and/or along multiple radial directions of the filter plate. In some embodiments, the filter plate 1610 can include grooves formed into the surface of the filter plate 1610 to assist in retention of cells and draining of solutions. In some embodiments, the grooves can form a concentric pattern.

In some implementations, a negative pressure is applied to the pod 105 via lower portion 1615, which can cause the cell suspension medium and/or delivery solution to be drained through the holes and through the lower portion 1615 while the cells are collected on filter surfaces.

In some embodiments, the pod 105 can include one or more sensors configured to measure a temperature or a pH of the cells or fluids provided within the pod 105. A colorimetric transducer may be introduced to read media color, dyes or indicators used within experiments. A special calibration pod may contain a force sensor to measure the force of atomized reagents landing on the cell surface on the pod. Additional calibration pods may have indicator paper (e.g., litmus) or TeeJay papers to evaluate the atomization spray. The sensors can be located, for example, on or within upper portion 1605, filter plate 1610, and/or lower portion 1615. In some embodiments, the pod 105 can include a memory and/or a communications module, such as a near-field communication module capable of transmitting experimental data associated with the processing of the pod 105.

In some implementations, the pod can include a microprocessor or controller to measure the time that the pod has been within the device. The microprocessor can store serial number for the pod and remember the serial number of the device into which it was placed. In the event that there is more than one device, a plurality of devices, where a pod moves from device to device to undergo sequential transfer actions, the microprocessor can store the sequence and serial numbers, timings and other information communicated from the device to the pod. Whether one or more devices are used, the device can transfer the process parameters for a given experiment along with environmental conditions, time/date, and the like to the pod. The information stored in permanent storage (e.g., EEPROM) can be read back by either another device or a pod reader.

As shown in FIG. 16C, the filter plate coupling 1615 can include a gripping surface configured about the circumference of the filter plate coupling 1615. The filter plate coupling 1615 can also include a flanged portion to couple with the bottom surface of the filter plate 1610. Additionally the filter plate coupling 1615 can include a hole 1620 for fluid to drain therethrough from the pod 105.

FIG. 16D shows another example pod implementation in which the upper portion 1605, filter plate 1610 are integral. A tube 1625 is attached to an opening feature on the underside of the filter plate coupling 1615, such as the hole 1620 shown in FIG. 16C.

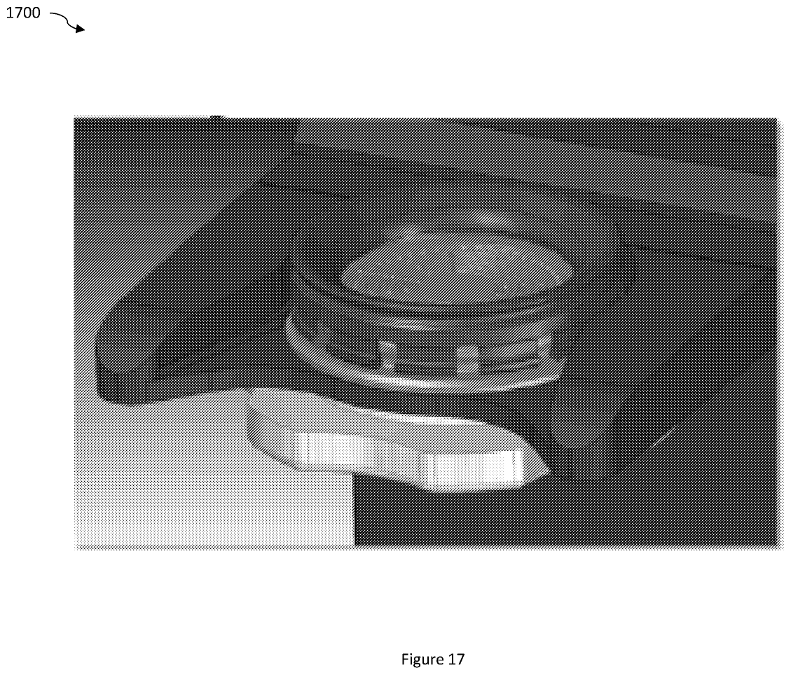

FIG. 17 is an isometric view of a CAD drawing illustrating an exemplary embodiment of a pod assembly within a pod nest of the delivery platform 100 of FIG. 1.

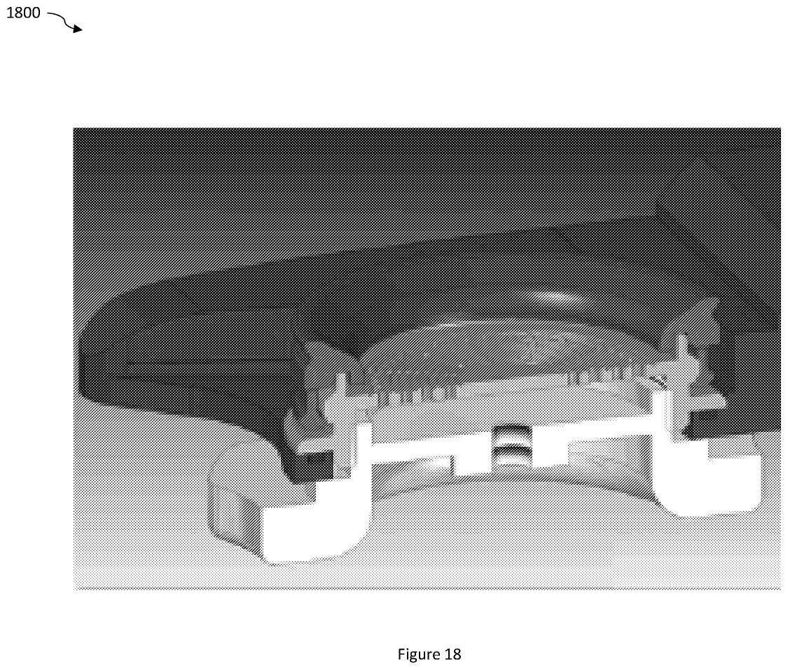

FIG. 18 is an cross-sectional view of the exemplary embodiment shown in FIG. 17.

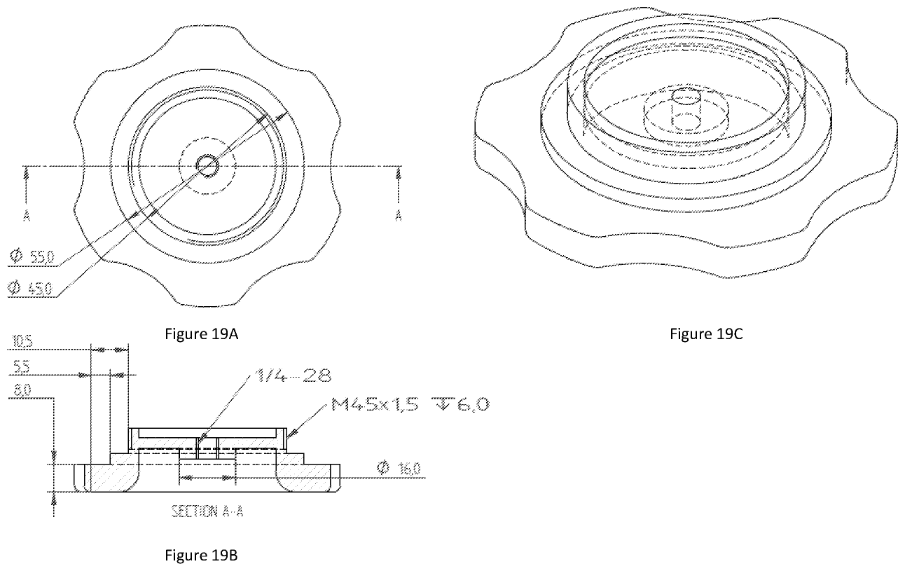

FIGS. 19A-19C are CAD drawings illustrating example embodiments of the filter plate coupling of the pod assembly 1500 shown in FIG. 15. The filter plate coupling 1615 shown in FIGS. 19A-19C corresponds to the filter plate coupling 1615 shown in FIG. 16C. The dimensions of the filter plate coupling 1615 shown in FIGS. 19A-19C are exemplary and not intended to limit the size or configuration of the filter plate coupling 1615.

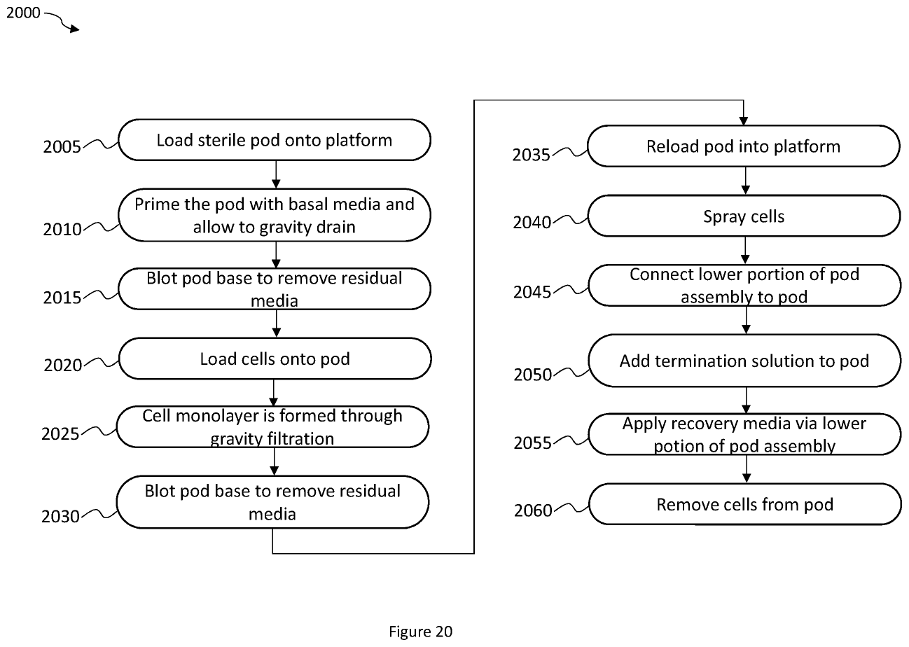

FIG. 20 is a flow diagram illustrating an example embodiment of a process for delivery of payload to cells using the delivery platform 100 of FIG. 1.

In operation, the target cells may be mixed in a medium at a particular concentration. For example, about 60 million cells may be mixed in about 60 mL medium. The prepared cell-containing medium may be introduced into the pod 105 via a disposable tube set and/or sterile needle/cannula. The loading procedure may be performed manually, or may be performed automatically using a pump (e.g., peristaltic pump or positive displacement pump) and a controller, as described boave. Similarly, the valve operation may be performed manually, or may be performed automatically using, for example, a solenoid valve and a controller. After the valves are closed, in some implementations, the medium displaces through the filter of the pod 105 by reason of gravity. In some implementations, a vacuum pressure is supplied to the lower portion 1615 of the pod 15 through the port of the lower portion. Accordingly, the medium is displaced through the filters, thereby depositing the target cells (e.g., T cells) on the filter surfaces. A beaker or other container may be used, for example, to collect the medium below the pod nest 110. In some implementations, a positive pressure and the vacuum pressure may be alternatingly supplied to the lower portion of the pod during the discharge of the medium to adjust/rearrange the cell deposition on the filters.

Subsequently, the delivery solution containing the payload (e.g., cargo) is sprayed via the atomizer. The controller may control the amount and duration of the spray. For example, the delivery solution may be sprayed for about 300 ms. For spraying the delivery solution, the cargo may be introduced to the spray head via microvial or injected via resealable injection port.

After the delivery solution is sprayed, a stop solution can be introduced via a disposable tube set and/or sterile plastic needle/cannula. The stop solution may be supplied manually, or may be supplied automatically using the pump and the controller. A desired amount of stop solution is introduced into the chamber. For example, about 10 mL of stop solution may be introduced over about 20 seconds. In some implementations, no stop solution is introduced to the cells.

Following the introduction of the stop solution, the cells are resuspended. For the resuspension, about 60 mL medium, which may be a used, new medium or the medium that was previously drained from the chamber, can be introduced by a syringe or a pump. The duration for the resuspension step may be about 1 minute. To improve resuspension, various methods such as tilting of the platform, agitation (e.g., vibration of the platform), and the like may be used during the resuspension process or after the resuspension process.

After the cells are resuspended in the medium, the engineered cells are collected for further processes. The pod may be flushed or washed after the process for subsequent procedures. Alternatively or additionally, the entire pod or a part of the pod may be made as a disposable unit that can be disposed after a use and replaced with a new one. FIG. 20 shows an exemplary process 2000. The process 2000, however, is not limited to operations shown in FIG. 20, and the process parameters, such as amount (volume) of medium, number of cells, concentration, duration for each step, may be varied depending on applications.

With reference to FIG. 20, at 2005, a sterile pod can be loaded onto the platform. At 2010, the pod can be primed with basal media and gravity can be allowed to drain the pod. At 2015, the pod base can be blotted to remove residual media. At 220 cells can be loaded onto the pod. At 2025, a cell monolayer can be formed through gravity filtration. At 2030, the pod base can be blotted to remove residual media. At 2035, the pod can be reloaded into the platform. At 2040, the cells can be sprayed by the platform with the delivery solution. At 2045, the lower portion of the pod assembly can be connected. At 2050, termination solution can be added to the pod. At 2055, recovery media can be applied via the lower portion. At 2060, cells can be removed from the pod.

The exemplary embodiments described in the Example 1 section can transfect from about 0.5 million to 15 million cells in a single transfection. The platform can allow consistent delivery of cargos, such as mRNA and the like, to T cells. The system may be enclosed within a biosafety cabinet for a sterile operation. The operation of the system may be performed manually or automatically. For the automated operation, the fluid handling system can be controlled automatically via the controller and control software. The platform may be configured as a multiple-use system which can be reused after cleaning and washing. In some implementations, the platform may be configured as a single-use, disposable system which includes disposable parts such as a disposable pod.

### Example 2

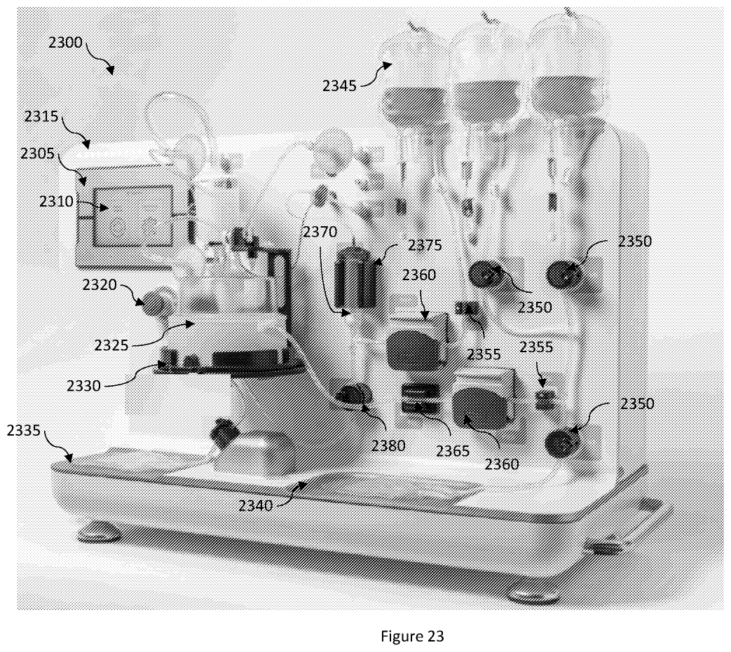

FIG. 23 illustrates an image of another example embodiment of a delivery platform 2300 according to some embodiments disclosed herein. In some implementations, the delivery platform 2300 can operate as a closed system in which cell processing experiments and production can occur within a sterile, sealed environment with reduced risk of contamination. In some embodiments, the delivery platform 2300 can be configured to perform some or all of the steps of process 2000 for delivery to cells as shown and described in relation to FIG. 20. In such embodiments, the pod 105 can be considered equivalent with the filter 2715 configured within the chamber assembly 2325.

As shown in FIG. 23, in some implementations, the delivery platform 2300 can include an instrument housing mounted to a base. The base can enable the delivery platform 2300 to be located or mounted on a bench, within a vented hood workspace, a desktop, a workbench, or the like. In some embodiments, the delivery platform 2300 can be mounted on a mobile base configured to transport the delivery platform 2300 from one location to another. Some configurations, such as mobile configurations, of the delivery platform 2300 can be positioned in proximity of a patient, for example, to more readily receive cell volumes directly from a patient and/or provide cells that have undergone delivery of a payload directly to the patient without requiring multiple handling steps, which can potentially introduce contamination.

As shown in FIG. 23, the delivery platform 2300 can include a display 2305 providing a human-machine interface (HMI) 2310. The HMI 2310 can be configured to receive user inputs associated with operation of the delivery platform 2300 and to provide outputs associated with the operations of the delivery platform 2300. In some embodiments, the HMI 2310 can be configured with one or more workflows, which can be initiated, performed, and/or stopped based on user-interaction with the HMI 23210. Individual processing workflows can be performed in a variety of non-limiting sequences based on broader user-defined workflows associated with a particular cell type, a particular reagent medium, and/or experimental application or objective. The HMI 2310 can be electrically coupled to one or more controllers configured within the delivery platform 2300. The delivery platform 2300 can also include one or more lights or visual indicators 2315 to indicate one or more statuses associated with operation of the delivery platform 2300. As shown in FIG. 23, the lights 2315 can include a plurality of lights, which can be individually operated with regard to one or more steps or procedures associated with operation of the delivery platform 2300. In some embodiments, the HMI 2310 can display one or more error or operational codes associated with an operation of the delivery platform 2300 and the lights 2315 can provide a user with a visual indication corresponding to the codes.

As further shown in FIG. 23, the delivery platform 2300 can include a stop button 2320. The stop button 2320 can cease operation of the delivery platform 2300 in the event of user error or operational error when operating the delivery platform 2300.

As further shown in FIG. 23, the delivery platform 2300 can include a chamber assembly 2325 mounted within a frame 2330. Advantageously, the chamber assembly 2325 can enable experiments to be performed in a variety of experimental processes without risk of contamination that can occur via re-usable assemblies. For example, the experimental processes can include a cell wash process, a cell concentration change process, and a cell medium change process. Thus, the accuracy of experimental results can be improved for different cell treatment processes compared to systems utilizing re-usable assemblies. The chamber assembly 2325 can be provided for use in a sealed, sterile packaging. The chamber assembly 2325 can include a filter upon which cells can be provided for delivery of payload and collected following delivery. In some embodiments, the filter can be a gas-permeable and liquid permeable filter. In some embodiments, the fresh reagent media can be introduced from below the filter, such as during washing workflows. The chamber assembly 2325 can be configured within the frame 2330. In some embodiments, the frame 2330 can be a semi-circular frame or a “C”-shaped frame. The frame 2330 can be mounted to shaft extending from the delivery platform 2300. The frame 2330, shown in a horizontal orientation in FIG. 23, can be configured to tilt in an upward or downward vertical direction by rotation of the shaft. For example, in some embodiments, the frame 2330 can be configured to tilt 0-10, 5-15, 10-20, 15-25, 20-30, or 25-45 degrees from the horizontal orientation shown in FIG. 23. In some embodiments, the shaft can be configured to tilt the frame 2330 in an oscillating manner with respect to an amount of angular tilt of the frame 2330. For example, the frame 2330 can be tilted to +30 degrees and the frame 2330 can then oscillate between a positive angular orientation (e.g., +1 degree) and a negative angular orientation (e.g., ˜1 degree) relative to the +30 degree orientation causing the frame 2330 to oscillate between +31 degrees and +29 degrees. The frame 2330 can oscillate between the two angular orientations with a predetermined or user-defined frequency. In some embodiments, the frame 2330 can oscillate at a frequency of 0.5 kHz, 1 kHz, 1.5 kHz, 2 kHz, 2.5 kHz, or more. In some embodiments, the shaft and/or the frame 2330 can be coupled to a servo motor configured to vibrate the shaft and/or the frame 2330.

Oscillating and/or vibrating the frame 2330 can advantageously increase the amount of cells collected following delivery of a payload compared to aspiration-based collection methods. Aspiration-based collection methods require repeated application and extraction of a collection media within the chamber assembly 2325. In addition, oscillating and/or vibrating the frame 2330 can also advantageously increase the viability of the collected cells, which can be reduced due to exposure to repeated fluid pressures and flow dynamics when cells are collected using aspiration-based collection methods.

As further shown in FIG. 23, the delivery platform 2300 can include a waste collection tray 2335 at which reagents and/or media evacuated from the chamber assembly 2325 can be collected. In some embodiments, the waste collection tray 2335 can be removed from the delivery platform 2300. As further shown in FIG. 23, the delivery platform 2300 can also include a cell collection tray 2340 at which cells that have undergone delivery of payload can be collected. In some embodiments, the cell collection tray 2340 can be removed from the delivery platform 2300. In some embodiments, the cell collection tray 2340 can include a cooling element and/or a heating element to maintain the cells at a desired temperature. In some embodiments, the heating and/or cooling elements associated with the cell collection tray 2340 can be configured within the base of the delivery platform 2300. In some embodiments, the base can include a scale located underneath the waste collection tray 2335 and/or the cell collection tray 2340. In this way, the delivery platform 2300 can determine a weight of collected media materials and collected cells. In some embodiments, the collection tray 2340 can include an articulating cradle in which media materials or collected cells can be held and maintained in motion to improve cell viability.

As further shown in FIG. 23, the delivery platform 2300 can include one or more media materials 2345. The media materials 2345 can be fluidically coupled to the chamber 2330 via one or more fluid circuits. The delivery platform 2300 can also include one or more valves 2350 configured to control an amount of media provided via the one or more fluid circuits. In some embodiments, the one or more values 2350 can include pinch valves. The delivery platform can include one or more fluid detection sensors 2355 configured in-line with respect to a corresponding fluid circuit. The fluid detection sensors 2355 can be configured to aid priming as well as calibration of the delivery platform 2300. In addition, the fluid detection sensors 2355 can be configured as a measurement system to calculate a volume of media within the fluid circuit between two locations. As further shown in FIG. 23, the delivery platform 2300 can include one or more pumps 2360 to pump cell culture media into the chamber assembly 2325. By pumping cell culture media into and out of the chamber assembly 2325 in a cyclic manner, while the frame 2330 is being vibrated and/or tilted, cell collection can be increased compared to non-tilting, non-vibrating cell collection operations. In some embodiments, the pumps 2360 can include peristaltic pumps. Other example pump types can include syringe pump, plunger-less syringe pump, closed syringe types, bag squeezer pump, and the like. The delivery platform 2300 can also include an ultrasonic flow rate detector 2365.

As further shown in FIG. 23, the delivery platform 2300 can include a syringe 2370. In some embodiments, the syringe 2370 can include a plunger-less syringe. Air can be applied to the syringe 2370 to provide the media 2345 to the chamber assembly 2325. The delivery platform 2300 can also include an optical sensor 2375 configured within a holder of the syringe 2370 or within the delivery platform 2300 itself. The optical sensor 2375 can detect a level of fluid within the syringe 2370 or the position of a plunger or a bung of the syringe 2370. The optical sensor 2375 can include an array of optical sensors, such as infra-red detectors, arranged linearly in a vertical array. The optical sensor 2375 can be used in calibration operations in combination with the pump 2360. In some embodiments, the syringe 2370 can be coupled to a check valve located at an exit of the syringe 2370. The optical sensor 2375 can be coupled to a valve 2380 to control an amount of media provided to the chamber assembly 2325.

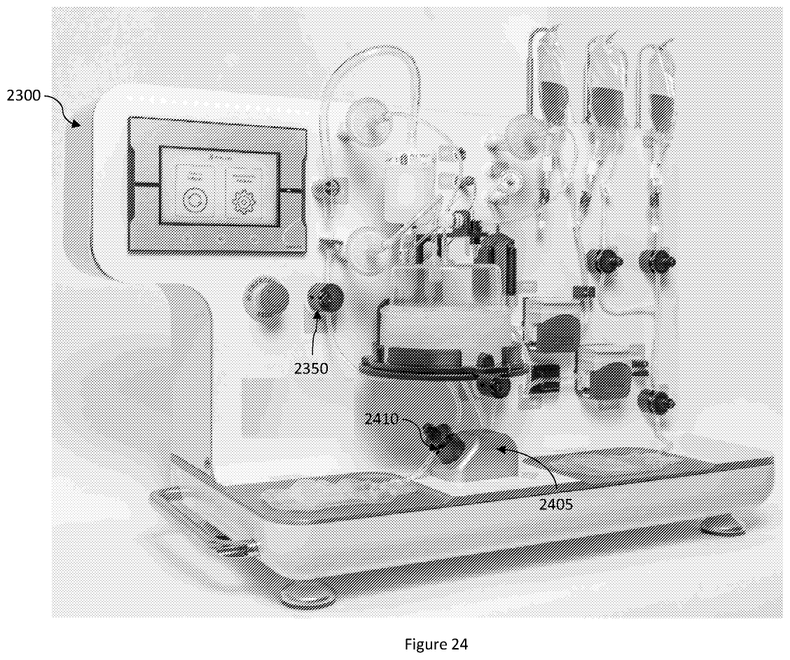

FIG. 24 illustrates a view of the delivery platform 2300 shown in FIG. 23. As shown in FIG. 24, the delivery platform 2300 can include a valve holder 2405 configured to hold a valve 2410. The valve holder 2405 can be configured to hold the valve 2410 at an angle relative to an orientation of the chamber assembly 2325.

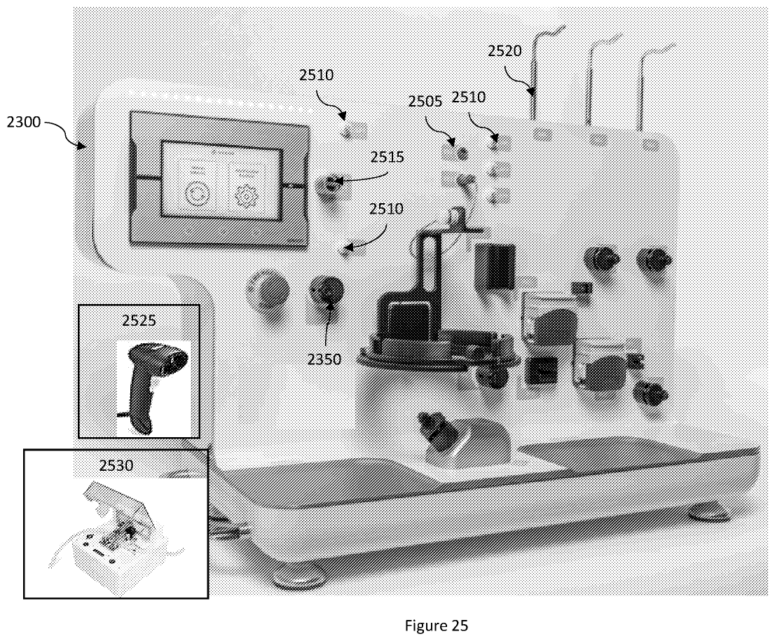

FIG. 25 illustrates a second view of the delivery platform shown in FIG. 23. As shown in FIG. 25, the delivery platform 2300 can include one or more electrical connectors 2505. In some embodiments, the electrical connectors 2505 can be instrument connectors to connect external instrumentation equipment to the delivery platform 2300. The external instrumentation can include, for example, an electrical thermometer, a hydrometer, a barometer, photoplethysmograph sensor, load cells, biochemical sensor (e.g., an alcohol sensor), optical sensor, transducer to measure vibration (e.g., vibrating membrane microelectronic machine (MEMs)), and the like.

The delivery platform 2300 can also include one or more gas connectors 2510. The gas connectors 2510 can receive a gas supply and provide the gas supply to the chamber assembly 2325 under desired pressure conditions via a gas circuit coupling the chamber assembly 2325 and the gas supply. In some embodiments, the gas connectors 2510 can receive a gas from the chamber assembly 2325, for example when purging or venting the chamber assembly 2325. In some implementations, the gas connectors 2510 can be independently controlled via software, and each gas connector 2510 can be configured to provide a static or dynamic head of pressure (e.g., a pressure set point). In some implementations, the gas connectors 2510 can operate with different gases (e.g., medical, nitrogen, and the like), can be software configurable, can provide contiguous airflow at a specified pressure into the vessel, and the like. Pressure can be provided by a flow control regulator, pressure regulator, flow transducer, pressure transducer, and the like. Pressure can be provided to other components such as an atomizer, a shower head, an Eppendorf needle, to drive the bung in a plungerless syringe, and the like. Each gas connector 2510 can be can be independently software configurable and not part of a manifold.

Valve 2350 can be coupled to a gas circuit associated with one of the gas connectors 2510 (as shown in FIG. 24) and can control an amount of gas supplied to the chamber assembly 2325. The delivery platform 2300 can include a hose clamp 2515 to secure a portion of hose configured for use with the chamber assembly 2325. The delivery platform 2300 can also include one or more hangers 2520 to hold media 2345. In some embodiments, the hangers 2520 can be configured with scales to determine a weight of the media 2345.

As shown in FIG. 25, the delivery platform 2300 can include a bar code reader 2525. The bar code reader 2525 can be configured to scan bar-coded media, a badge associated with an operator of the delivery platform 2300, and/or bar-coded packaging containing the chamber assembly 2325. In some embodiments, the bar code reader 2525 can include a linear bar code reader or 2-D bar code reader. In some embodiments, the bar code reader 2525 can be a hand-held bar code reader. In some embodiments, the HMI 2310 can be communicatively coupled to the bar code reader 2525. Additionally, the delivery platform 2300 can include a tube welder 2530 configured to fix or apply a weld to tubing of the delivery platform 2300, such as tubing used in association with the one or more fluid circuits coupled to the media 2345. In some embodiments, the HMI 2310 can be communicatively coupled to the tube welder 2530.

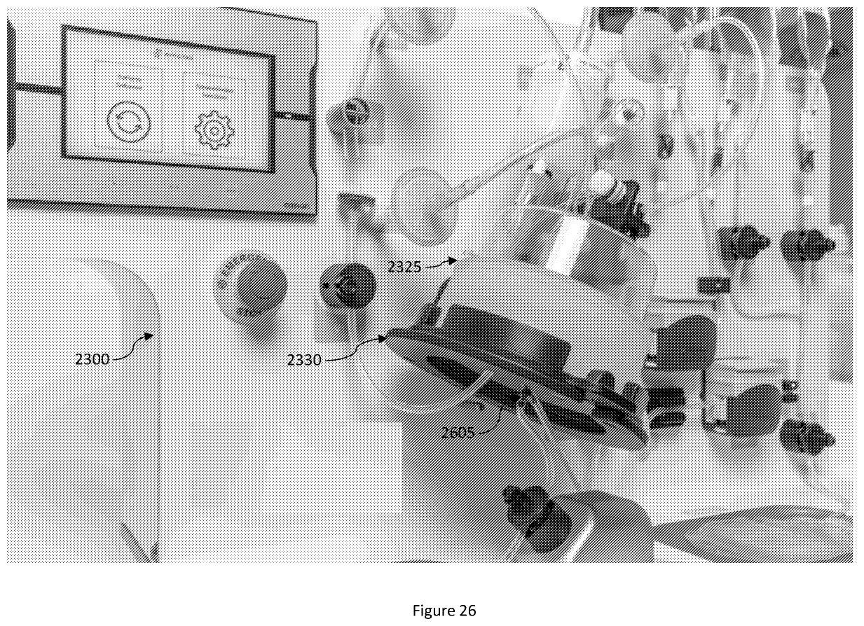

FIG. 26 illustrates a close-up view of a portion of the delivery platform shown in FIG. 23. As shown in FIG. 26, the chamber assembly 2325 and the frame 2330 are tilted at about a +30 degree angular orientation relative to the horizontal orientation shown in FIG. 23. In this position, the frame 2330 can oscillate in positive and negative angular movements from the +30 degree angular orientation to aid collection of cells via a drain 2605 configured in the bottom of the chamber assembly 2325. In some embodiments, the chamber assembly 2325 can be enclosed, at least partially, in an insulative or conductive jacket to provide heating or cooling to the chamber assembly 2325.

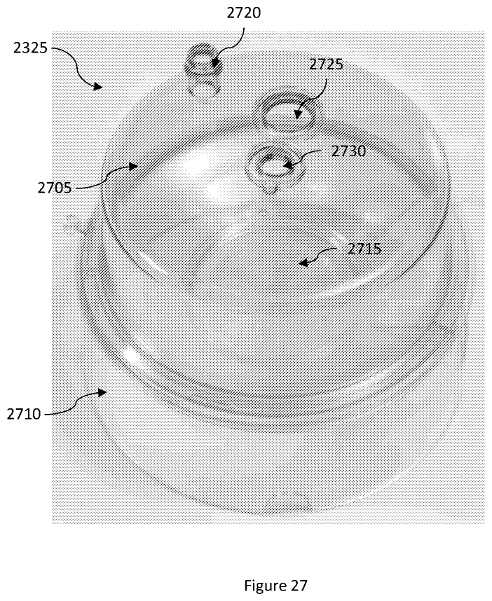

FIG. 27 illustrates an image of an example embodiment of a chamber assembly 2325 of the delivery platform shown in FIG. 23. In some embodiments, one or more inner surfaces or regions of the chamber assembly 2325 can be coated or patterned to aid cell mobility and/or adherence. For example, the chamber assembly 2325 can include one or more three-dimensional structures formed on one or more surfaces or regions within the chamber assembly 2325. Additional example three-dimensional structures include circumferential ribs forming a grove between adjacent ribs, spirals, radial ribs, bumps, dimples, hatch patterns, and the like. In some implementations, circumferential ribs can have a rib having a triangular profile with about 500 microns on each side, with a spacing between groves of 2 mm.

In some implementations, the patterns can control the flow of cells in culture medium during the filtration process. Such patterns can direct flow towards or away form areas on the filter surface, to counteract fluid forces that tend to cause bulging of the filter in the center and even-out the concentration of cells deposited on the filter surface.

A variety of non-limiting coating material may be applied to the inner surfaces of the chamber assembly 2325. In this way, the chamber assembly 2325 can provide surfaces for cell adherence, which can aid delivery, permeabilization, and/or cell collection. In some implementations, the internal chamber surfaces may be hydrophobic (e.g., made of polycarbonate for clarity of visualization) and is filter hydrophilic. In some embodiments, the chamber assembly 2325 can include one or more removable and replaceable portions, which can be swapped in or out of use. For example, a removable portion acting as a mask can be swapped in to the chamber in order to expose a smaller portion of the filter membrane. A benefit of this can include enabling the transfection of cells in small numbers e.g., less than 10\hat{ }6, less than 2×10\hat{ }6, less than 5×10\hat{ }6 and/or less than 10\hat{ }7. Cell types that can benefit include tumor-infiltrating lymphocytes (TILs), human stem cells (HSCs) and induced pluripotent stem (IPS) cells.

As shown in FIG. 27, the chamber assembly 2325 can include an upper portion 2705 and a lower portion 2710. The upper portion 2705 can be removable from the lower portion 2710. The lower portion 2710 can include a filter 2715 upon which cells can be deposited, permeabilized, and collected from. In some embodiments, the filter 2715 can be coated and or include a patterned material suitable for aiding cell adherence to the filter 2715. For example, the coating materials may be beneficial to reduce adherence for suspension cells, increase mobility of cells across the filter surface. This is beneficial in increasing viability (and yield) of cells that can more easily be recovered from the filter surface after transfection. A greater wet-ability also spreads the cargo and solution over the surface, thereby contacting more cells and increasing transfection. Examples of coating materials include polyvinylpyrrolidone (PVP) deposited on the material as a wetting agent. Such an agent allows (more easily allows) wetting and spreading of cells in medium within the chamber. A similar result may achieved by sputtering with Au (gold), oxygen plasma treatment, reduced surface charge or other strategies to render the filter surface more hydrophilic. Similarly, with adherent cells such as MSC, IPS, A549, HEK293, and macrophages, there may be a benefit to have the filter surface hydrophilic

A variety of non-limiting filters 2715 can be used within the chamber assembly 2325 depending on cell types and/or experimental or therapeutic applications. The upper portion 2705 can include a gas port 2720 configured to receive a gas via the gas connectors 2510. The upper portion 2705 can also include an air diffuser opening 2725 configured to receive an air diffuser coupled to gas connectors 2510. The air diffuser can be configured to alter static air conditions within the chamber assembly 2325 and to pressurize the chamber assembly 2325 for use as a closed system. The air diffuser can supply gases or combinations of gases into the chamber assembly 2325 under various temperature and pressure conditions. For example, the air diffuser can provide a gas comprising a particular concentration of CO₂ gas. As further shown in FIG. 27, the upper portion 2705 can include a spray head opening 2730 configured to receive a spray head therein.

The chamber assembly 2325 can be configured in a variety of non-limiting sizes and volumes. In some embodiments, the chamber assembly 2325 can have a volume of about 1 L. In some embodiments, the lower portion 2710 can have a volume of about 300-500 ml.

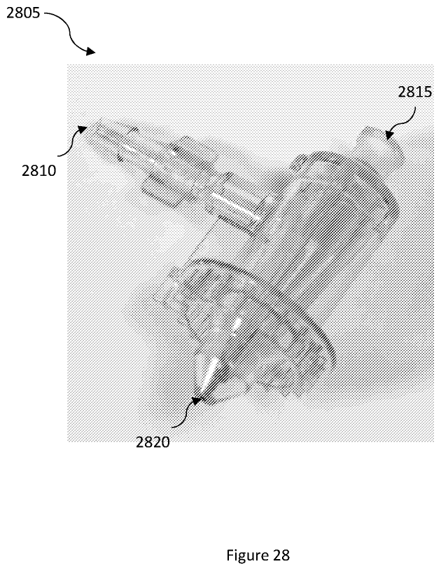

FIG. 28 illustrates an image of an example embodiment of a spray head of the single-use assembly shown in FIG. 23. As shown in FIG. 28, a spray head 2805 can include a gas inlet port 2810 and a fluid inlet port 2815. The gas inlet port 2810 can be coupled to any of gas connectors 2510 to supply a pressurized gas within the chamber assembly 2325. The fluid inlet port 2815 can be coupled to a supply of isotonic aqueous solution that includes a payload. A pressurized spray can be formed within the spray head 2805 and delivered into the chamber assembly 2325 via the outlet 2820.

Due to the process automation, the cell engineering platform can perform the cell treatment and manipulation processes, such as transfection, more consistently, and cargo delivery can be performed more easily. Accordingly, the cell engineering platform can provide reliable vector-free delivery method to reduce the cost and complexity of the cell engineering technologies.

Although a few variations have been described in detail above, other modifications or additions are possible. For example, design variations can include chamber assemblies 2325 or filters 2715 of other geometries, e.g., rectangular, square or elliptical. Additionally, chamber assemblies 2325 or filters 2715, with varying topography, can include convex, concave and textured surfaces with micro or macro features. Also, target configurations including both circular targets and annular targets are contemplated. In embodiments, the modifications or additions can optimize cell deposition under the spray target. Although the terms chamber assembly 2325 or single-use assembly is used herein, in some implementations, the chamber assembly 2325 can be reusable (e.g., can be used more than once, for example, to process multiple populations of cells).

The subject matter described herein provides many technical advantages. For example, chamber assemblies as described herein avoid the need for sterilization of the system and greatly reduces the risk of cross contamination between patient samples, and enabling a simpler validation process. Another advantage is that the chamber assemblies described herein enable the delivery of multiple cargos through co-delivery through a single spray head. Furthermore, the subject matter described herein is fast and simple, and the gentle cell processing maintains cell health and enables engineering of naïve cell populations.

### Example 3

In some implementations, the delivery platform 2300 can be utilized for cell processing functionality in addition to delivery of a payload (e.g., transfection). For example, the delivery platform 2300 can be utilized to enable a variety of upstream and/or downstream cell processing workflows using the chamber assembly 2325. As used herein, upstream cell processing includes processes that are performed prior to delivery of a payload using the above-described process (e.g., contacting the cell population with a solution including a payload (e.g., via spray)), and downstream cell processing includes processes that are performed after delivery of a payload using the above-described process (e.g., contacting the cell population with a solution including a payload (e.g., via spray)). As an example, the chamber assembly 2325 can be utilized as a bioreactor for cell culture (e.g., incubation) of the population of cells after the above-described delivery process is performed (e.g., contacting the cell population with a solution including a payload (e.g., via spray)). By utilizing the chamber assembly 2325 for additional cell processing steps, cell viability can be improved.

In order to utilize the delivery platform 2300 as an incubator, the delivery platform 2300 can provide for environmental control to maintain the population of cells within an artificial environment favorable for incubation. Ideal culture conditions can vary widely for different cell types, but the artificial environment in which the cells are cultured can include the chamber assembly 2325 as the vessel with the filter as a substrate and/or additional medium can be applied that supplies the essential nutrients (amino acids, carbohydrates, vitamins, minerals, and the like), growth factors, hormones, and gases (oxygen (O₂), carbon dioxide (CO₂), Nitrogen (N₂), and the like), and regulates the physicochemical environment (pH, osmotic pressure, temperature).

The delivery platform 2300 can include a chamber assembly 2325 configured as a closed chamber that can enable introduction and control of sterile gases under user-defined pressure and temperature settings. For example, the delivery platform 2300 can be configured to introduce, circulate, and evacuate gases and compositions of gases within the closed, sealed chamber. Examples of the gases and compositions of gases can include clinical air, nitrogen, and gases and combinations of gases associated with workflows to collect, preserve, and/or produce cells which have undergone delivery of payload using the delivery platform 2300. Temperature within the closed chamber can also be controlled according to user-defined settings by adjusting the temperature of gases supplied into the closed chamber and/or external application of heat to the closed chamber. The delivery platform 2300 can further provide motion or non-motion of the closed chamber according to user-defined procedures. Thus, a number of experimental and production parameters can be adjusted with the closed, sealed configuration of the delivery platform 2300 to increase counts of viable cells.

Environmental control can include controlling the gas composition of the environment, the temperature of the environment, motion of the environment, and composition of mediums introduced into the environment. The delivery platform 2300 can control the gas composition of the environment, for example, by applying an appropriate mixture of gas via the gas diffuser to the chamber assembly 2325. For example, the delivery platform 2300 can control the environment within the chamber assembly 2325 to have a specific composition including controlling the concentration of gasses such as carbon dioxide (CO₂), nitrogen (N₂), and oxygen (O₂). For a given growth medium included in the single-use assembly, the growth medium controls the pH of the culture and buffers the cells in culture against changes in the pH. This buffering can be achieved by including an organic (e.g., HEPES) or CO₂-bicarbonate based buffer. Because the pH of the medium is dependent on the balance of dissolved carbon dioxide (CO₂) and bicarbonate (HCO₃⁻), changes in the atmospheric CO₂ can alter the pH of the medium. Therefore, the delivery platform 2300 can control environmental CO₂ concentration when using media buffered with a CO₂-bicarbonate based buffer. The CO₂ concentration can be provide at between 1%-10%, 5-7%, or 4-10% CO₂ in air. For example, CO₂ concentrations can be maintained at 1, 2, 3, 4, 5, 6, 7, 8, 9, or 10%.

The delivery platform 2300 can maintain the population of cells at a suitable temperature. This can be achieved, for example, by including a heating element within the chamber assembly 2325. The heating element can be any suitable type, for example, an electric heating element. In some implementations, a conductive sleeve can be included to aid in heat transfer and maintaining the cell population at a uniform temperature. The temperature can vary based on a particular application and population of cells. For example, the temperature of a cell population can be maintained at the body temperature of the host from which the cells were isolated, and to a lesser degree on the anatomical variation in temperature (e.g., temperature of the skin may be lower than the temperature of skeletal muscle). Overheating can be a more serious problem than under heating for cell cultures; therefore, in some cell culture protocols, the temperature can be set slightly lower than the optimal temperature. Many human and mammalian cell lines are maintained at 36° C. to 37° C. for optimal growth, although other temperatures are possible. In some implementations, temperature can be controlled by controlling a temperature of the gas introduced into the environment.

The delivery platform 2300 can control motion of the environment. For example, the delivery platform 2300 can rock or oscillate the chamber assembly 2325 as described in more detail above. The rocking or oscillation can be performed for the duration of the cell culture, or as required by a given cell processing protocol.

The delivery platform 2300 can control the media contained within the chamber assembly 2325. For example, media such as an organic (e.g., HEPES) or CO₂-bicarbonate based buffer can be introduced into the chamber assembly via a port. In some implementations, the media can be introduced via the syringe 2370 to provide media (such as media 2345) to the chamber assembly 2325. In some implementations, to prevent the culture media from draining from the chamber assembly 2325, a pinch valve or clip can be included on the tubing below the single-use assembly to maintain the media in the assembly for a longer period of time.

When utilizing the chamber assembly 2325 for cell culture, cells can be seeded at a density to allow for growth.

In some implementations, cell metabolites can be analyzed during cell culture. For example, glucose, glutamine, lactate and CO₂ can be monitored as part of controlling the changing of culture medium during the cell culture process. In some implementations, different culture mediums can be introduced and/or removed as desired.

In some implementations, cell samples can be removed from the population of cells, for example, for testing during the culture process. By enabling removal of cells during cell culture, additional measurements and/or testing can be performed on a cell culture over a time period. In some implementations, cells can be removed and placed into an external bio-reactor (e.g., another pod or chamber suitable for cell culture). In some implementations, cell removal can be achieved via extraction through one or more tubes.

In some implementations, the cells can be stirred during the cell culture. In some implementations, the stirring can be performed manually. In some implementations, the stirring can be performed by the delivery platform 2300, such as via oscillating the chamber assembly 2325. Vibrational perturbations in the frequency range 50 Hz to 2500 Hz and physical excursions (e.g., amplitude) of 2 mm or more may provide appropriate mixing or agitation. In some implementations, lower or higher frequencies (e.g., 1-50 Hz, or 2500-5000 Hz) and greater amplitude (e.g., 2-20 mm) can be used.

As another example of a downstream process the delivery platform 2300 can be configured to perform, the delivery platform 2300 can be utilized for performing a cryopreservation process. For example, cryopreserving cultured cells can include storing them in liquid nitrogen in complete medium in the presence of a cryoprotective agent such as dimethylsulfoxide (DMSO) or glycerol. Cryoprotective agents reduce the freezing point of the medium and also allow a slower cooling rate, greatly reducing the risk of ice crystal formation, which can damage cells and cause cell death. Accordingly, a cryoprotective agent such as DMSO can be provided into the chamber assembly 2325, for example, using a port in the chamber assembly 2325 to aid in cryopreservation of the population of cells (e.g., cells after undergoing one or more of the above-described processes such as delivery of a payload using a permeabilization agent (e.g., transfection), virus-based transduction, cell culture, and the like).

In some embodiments, the delivery platform 2300 can be configured to allow for introducing viral components to a volume of cells within the closed chamber to culture or co-culture the cells (e.g., a cell transduction process). For example, replication-deficient viruses containing genetic material to be introduced into the target cells can be added to the cell population before or after the above-described delivery process is performed (e.g., contacting the cell population with a solution including a payload (e.g., via spray)).

The delivery platform 2300 can further be configured for downstream processing such as washing, harvesting, and cryopreservation. In some embodiments, the delivery platform 2300 can be easily connected to other experimental or therapeutic devices, platforms, or systems. For example, the delivery platform 2300 can be fluidically coupled to other specialized or traditional permeabilization or cell processing platforms, such as an Eppendorf reactor.

The delivery platform 2300 can readily facilitate upstream processing, such as activation of cells and/or beads within the closed chamber, as well as priming of cells during transduction and/or transfection workflows. Cell washing and volume reduction can be performed as upstream processing steps. Other upstream processes can be used as well.

The delivery platform 2300 can advantageously provide a controllable, closed environment that is easily configured in experimental and application-specific settings and does not require expensive, complicated machinery or automated mechanisms to operate. The configuration of interchangeable mediums and collection vessels can enable flexible, adjustable workflows or process loops that are application or experiment-specific while maintaining sterile, uncontaminated operation of the delivery platform 2300. Frequently, contamination can be introduced via open systems, or movement/relocation of a platform. Contamination can also be introduced in systems that require connection and disconnection of multiple fluidic channels or conduits to introduce or extract cells and/or reagent mediums.

The subject matter described herein provides many technical advantages. For example, by utilizing the delivery platform 2300 for performing cell culture, some implementations can provide for excellent model systems for studying the normal physiology and biochemistry of cells (e.g., metabolic studies, aging), the effects of drugs and toxic compounds on the cells, and mutagenesis and carcinogenesis. It can also be used in drug screening and development, and large scale manufacturing of biological compounds (e.g., vaccines, therapeutic proteins). By utilizing the delivery platform 2300 for performing cell culture, consistency and reproducibility of results can be improved. Moreover, by utilizing the delivery platform 2300 for performing cell culture, the cells do not need to be moved from the single use assembly to a separate vessel or culture well, which can improve viability and reduce contamination.

Although the terms upstream processing and downstream processing have been used herein, some implementations of the current subject matter can implement any process step in any desired order. For example, the processes can be user-defined and can be defined in any order (whether the steps occur upstream or downstream).

### Embodiments of Example Delivery Protocols

The invention is based on the surprising discovery that compounds or mixtures of compounds (compositions) are delivered into the cytoplasm of eukaryotic cells by contacting the cells with a solution containing a compound(s) to be delivered (e.g., payload). Preferably, the solution is delivered to the cells in the form of a spray, e.g., aqueous particles. (see, e.g., PCT/US2015/057247 and PCT/IB2016/001895, hereby incorporated in their entirety by reference). For example, the cells are coated with the spray but not soaked or submersed in the delivery compound-containing solution. In some implementations, the delivery solution can include an agent that permeabilizes or dissolves a cell membrane, although the agent may not be required to affect delivery of the payload the agent may enhance delivery. Exemplary agents that permeate or dissolve a eukaryotic cell membrane include alcohols and detergents such as ethanol and Triton X-100, respectively. Other exemplary detergents, e.g., surfactants include polysorbate 20 (e.g., Tween 20), 3-[(3-cholamidopropyl)dimethylammonio]-1-propanesulfonate (CHAPS), 3-[(3-cholamidopropyl)dimethylammonio]-2-hydroxy-1-propanesulfonate (CHAPSO), sodium dodecyl sulfate (SDS), and octyl glucoside.

An example of conditions to achieve a coating of a population of coated cells include delivery of a fine particle spray, e.g., the conditions exclude dropping or pipetting a bolus volume of solution on the cells such that a substantial population of the cells are soaked or submerged by the volume of fluid. Thus, the mist or spray comprises a ratio of volume of fluid to cell volume. Alternatively, the conditions comprise a ratio of volume of mist or spray to exposed cell area, e.g., area of cell membrane that is exposed when the cells exist as a confluent or substantially confluent layer on a substantially flat surface such as the bottom of a tissue culture vessel, e.g., a well of a tissue culture plate, e.g., a microtiter tissue culture plate.

“Cargo” or “payload” are terms used to describe a compound, or composition that is delivered via an aqueous solution across a cell plasma membrane and into the interior of a cell.

In an aspect, delivering a payload across a plasma membrane of a cell includes providing a population of cells and contacting the population of cells with a volume of an aqueous solution. The aqueous solution includes the payload. In some implementations, the aqueous solution includes no alcohol. In some implementations, the aqueous solution includes an alcohol content greater than 0.2 percent concentration. In some implementations, the aqueous solution includes the payload and an alcohol content greater than 5 percent concentration. The volume of the aqueous solution may be a function of exposed surface area of the population of cells, or may be a function of a number of cells in the population of cells.

In another aspect, a composition for delivering a payload across a plasma membrane of a cell includes an aqueous solution including the payload, greater than 46 mM salt, less than 121 mM sugar, and less than 19 mM buffering agent. In some implementations, the aqueous solution does not include any alcohol. In some implementations, the aqueous solution includes alcohol at greater than 0.2 percent concentration. For example, the alcohol, e.g., ethanol, concentration is greater than 2 percent, greater than 5 percent, and/or does not exceed 50%.

One or more of the following features can be included in any feasible combination. The volume of solution to be delivered to the cells is a plurality of units, e.g., a spray, e.g., a plurality of droplets on aqueous particles. The volume is described relative to an individual cell or relative to the exposed surface area of a confluent or substantially confluent (e.g., at least 75%, at least 80% confluent, e.g., 85%, 90%, 95%, 97%, 98%, 100%) cell population. For example, the volume can be between 6.0×10⁻⁷ microliter per cell and 7.4×10⁻⁴ microliter per cell. The volume is between 4.9×10⁻⁶ microliter per cell and 2.2×10⁻³ microliter per cell. The volume can be between 9.3×10⁻⁶ microliter per cell and 2.8×10⁻⁵ microliter per cell. The volume can be about 1.9×10⁻⁵ microliters per cell, and about is within 10 percent. The volume is between 6.0×10⁻⁷ microliter per cell and 2.2×10⁻³ microliter per cell. The volume can be between 2.6×10⁻⁹ microliter per square micrometer of exposed surface area and 1.1×10⁻⁶ microliter per square micrometer of exposed surface area. The volume can be between 5.3×10⁻⁸ microliter per square micrometer of exposed surface area and 1.6×10⁻⁷ microliter per square micrometer of exposed surface area. The volume can be about 1.1×10⁻⁷ microliter per square micrometer of exposed surface area. About can be within 10 percent.

Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel.

Contacting the population of cells with the volume of aqueous solution can be performed by gas propelling the aqueous solution to form a spray. The gas can include nitrogen, ambient air, or an inert gas. The spray can include discrete units of volume ranging in size from, 1 nm to 100 μm, e.g., 30-100 μm in diameter. The spray includes discrete units of volume with a diameter of about 30-50 μm. A total volume of aqueous solution of 20 μl can be delivered in a spray to a cell-occupied area of about 1.9 cm², e.g., one well of a 24-well culture plate. A total volume of aqueous solution of 10 μl is delivered to a cell-occupied area of about 0.95 cm², e.g., one well of a 48-well culture plate. Typically, the aqueous solution includes a payload to be delivered across a cell membrane and into cell, and the second volume is a buffer or culture medium (e.g., a stop solution) that does not contain the payload. Alternatively, the second volume (buffer or media) can also contain payload. In some embodiments, the aqueous solution includes a payload and an alcohol, and the second volume does not contain alcohol (and optionally does not contain payload). The population of cells can be in contact with said aqueous solution for 0.1 10 minutes prior to adding a second volume of buffer or culture medium to submerse or suspend said population of cells. The buffer or culture medium can be phosphate buffered saline (PBS). The population of cells can be in contact with the aqueous solution for 2 seconds to 5 minutes prior to adding a second volume of buffer or culture medium to submerse or suspend the population of cells. The population of cells can be in contact with the aqueous solution, e.g., containing the payload, for 30 seconds to 2 minutes prior to adding a second volume of buffer or culture medium, e.g., without the payload, to submerse or suspend the population of cells. The population of cells can be in contact with a spray for about 1-2 minutes prior to adding the second volume of buffer or culture medium to submerse or suspend the population of cells. During the time between spraying of cells and addition of buffer or culture medium, the cells remain hydrated by the layer of moisture from the spray volume.

The aqueous solution can include an ethanol concentration of 5 to 30%. The aqueous solution can include one or more of 75 to 98% H₂O, 2 to 45% ethanol, 6 to 91 mM sucrose, 2 to 500 mM KCl, 2 to 35 mM ammonium acetate, and 1 to 14 mM (4-(2-hydroxyethyl)-1-piperazineethanesulfonic acid) (HEPES). For example, the delivery solution contains 10⁶ mM KCl and 27% ethanol.

The population of cells can include adherent cells or non-adherent cells. The adherent cells can include at least one of primary mesenchymal stem cells, fibroblasts, monocytes, macrophages, lung cells, neuronal cells, fibroblasts, human umbilical vein (HUVEC) cells, Chinese hamster ovary (CHO) cells, and human embryonic kidney (HEK) cells or immortalized cells, such as cell lines. In preferred embodiments, the population of cells comprises non-adherent cells, e.g., the % non-adherent cells in the population is at least 50%, 60%, 75%, 80%, 90%, 95%, 98%, 99% or 100% non-adherent cells. Non-adherent cells primary cells as well as immortalized cells (e.g., cells of a cell line). Exemplary non-adherent/suspension cells include primary hematopoietic stem cell (HSC), T cells (e.g., CD3+ cells, CD4+ cells, CD8+ cells), natural killer (NK) cells, cytokine-induced killer (CIK) cells, human cord blood CD34+ cells, B cells, or cell lines such as Jurkat T cell line.

The payload can include a small chemical molecule, a peptide or protein, or a nucleic acid. The small chemical molecule can be less than 1,000 Da. The chemical molecule can include MitoTracker® Red CMXRos, propidium iodide, methotrexate, and/or DAPI (4′,6-diamidino-2-phenylindole). The peptide can be about 5,000 Da. The peptide can include ecallantide under trade name Kalbitor, is a 60 amino acid polypeptide for the treatment of hereditary angioedema and in prevention of blood loss in cardiothoracic surgery), Liraglutide (marketed as the brand name Victoza, is used for the treatment of type II diabetes, and Saxenda for the treatment of obesity), and Icatibant (trade name Firazyer, a peptidomimetic for the treatment of acute attacks of hereditary angioedema). The small-interfering ribonucleic acid (siRNA) molecule can be about 20-25 base pairs in length, or can be about 10,000-15,000 Da. The siRNA molecule can reduces the expression of any gene product, e.g., knockdown of gene expression of clinically relevant target genes or of model genes, e.g., glyceraldehyde-3phosphate dehydrogenase (GAPDH) siRNA, GAPDH siRNA-FITC, cyclophilin B siRNA, and/or lamin siRNA. Protein therapeutics can include peptides, enzymes, structural proteins, receptors, cellular proteins, or circulating proteins, or fragments thereof. The protein or polypeptide be about 100-500,000 Da, e.g., 1,000-150,000 Da. The protein can include any therapeutic, diagnostic, or research protein or peptide, e.g., beta-lactoglobulin, ovalbumin, bovine serum albumin (BSA), and/or horseradish peroxidase. In other examples, the protein can include a cancer-specific apoptotic protein, e.g., Tumor necrosis factor-related apoptosis inducing protein (TRAIL).

An antibody is generally be about 150,000 Da in molecular mass. The antibody can include an anti-actin antibody, an anti-GAPDH antibody, an anti-Src antibody, an anti-Myc ab, and/or an anti-Raf antibody. The antibody can include a green fluorescent protein (GFP) plasmid, a GLuc plasmid and, and a BATEM plasmid. The DNA molecule can be greater than 5,000,000 Da. In some examples, the antibody can be a murine-derived monoclonal antibody, e.g., ibritumomab tiuxetin, muromomab-CD3, tositumomab, a human antibody, or a humanized mouse (or other species of origin) antibody. In other examples, the antibody can be a chimeric monoclonal antibody, e.g., abciximab, basiliximab, cetuximab, infliximab, or rituximab. In still other examples, the antibody can be a humanized monoclonal antibody, e.g., alemtuzamab, bevacizumab, certolizumab pegol, daclizumab, gentuzumab ozogamicin, trastuzumab, tocilizumab, ipilimumamb, or panitumumab. The antibody can comprise an antibody fragment, e.g., abatecept, aflibercept, alefacept, or etanercept. The invention encompasses not only an intact monoclonal antibody, but also an immunologically-active antibody fragment, e.g.; a Fab or (Fab)2 fragment; an engineered single chain Fv molecule; or a chimeric molecule, e.g., an antibody which contains the binding specificity of one antibody, e.g., of murine origin, and the remaining portions of another antibody, e.g., of human origin.

The payload can include a therapeutic agent. A therapeutic agent, e.g., a drug, or an active agent”, can mean any compound useful for therapeutic or diagnostic purposes, the term can be understood to mean any compound that is administered to a patient for the treatment of a condition. Accordingly, a therapeutic agent can include, proteins, peptides, antibodies, antibody fragments, and small molecules. Therapeutic agents described in U.S. Pat. No. 7,667,004 (incorporated herein by reference) can be used in the methods described herein. The therapeutic agent can include at least one of cisplatin, aspirin, statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. The payload can include a diagnostic agent. The diagnostic agent can include a detectable label or marker such as at least one of methylene blue, patent blue V, and indocyanine green. The payload can include a fluorescent molecule. The payload can include a detectable nanoparticle. The nanoparticle can include a quantum dot.

The population of non-adherent cells can be substantially confluent, such as greater than 75 percent confluent. Confluency of cells refers to cells in contact with one another on a surface. For example, it can be expressed as an estimated (or counted) percentage, e.g., 10% confluency means that 10% of the surface, e.g., of a tissue culture vessel, is covered with cells, 100% means that it is entirely covered. For example, adherent cells grow two dimensionally on the surface of a tissue culture well, plate or flask. Non-adherent cells can be spun down, pulled down by a vacuum, or tissue culture medium aspiration off the top of the cell population, or removed by aspiration or vacuum removal from the bottom of the vessel. The population of cells can form a monolayer of cells.

The alcohol can be selected from methanol, ethanol, isopropyl alcohol, butanol and benzyl alcohol. The salt can be selected from NaCl, KCl, Na₂HPO₄, KH₂PO₄, and C₂H₃O₂NH. In preferred embodiments, the salt is KCl. The sugar can include sucrose. The buffering agent can include 4-2-(hydroxyethyl)-1-piperazineethanesulfonic acid.

The present subject matter relates to a method for delivering molecules across a plasma membrane. The present subject matter finds utility in the field of intra-cellular delivery, and has application in, for example, delivery of molecular biological and pharmacological therapeutic agents to a target site, such as a cell, tissue, or organ. The method of the present subject matter comprises introducing the molecule to an aqueous composition to form a matrix; atomizing the matrix into a spray; and contacting the matrix with a plasma membrane.

This present subject matter relates to a composition for use in delivering molecules across a plasma membrane. The present subject matter finds utility in the field of intra-cellular delivery, and has application in, for example, delivery of molecular biological and pharmacological therapeutic agents to a target site, such as a cell, tissue, or organ. The composition of the present subject matter comprises an alcohol; a salt; a sugar; and/or a buffering agent.

In some implementations, demonstrated is a delivery technique that facilitates intracellular delivery of molecules independent of the molecule and cell type. Nanoparticles, small molecules, nucleic acids, proteins and other molecules can be efficiently delivered into suspension cells or adherent cells in situ, including primary cells and stem cells, with low cell toxicity and the technique is compatible with high throughput and automated cell-based assays.

Some example methods described herein include a payload, wherein the payload includes an alcohol. By the term “an alcohol” is meant a polyatomic organic compound including a hydroxyl (—OH) functional group attached to at least one carbon atom. The alcohol may be a monohydric alcohol and may include at least one carbon atom, for example methanol. The alcohol may include at least two carbon atoms (e.g. ethanol). In other aspects, the alcohol comprises at least three carbons (e.g. isopropyl alcohol). The alcohol may include at least four carbon atoms (e.g., butanol), or at least seven carbon atoms (e.g., benzyl alcohol). The example payload may include no more than 50% (v/v) of the alcohol, more preferably, the payload comprises 2-45% (v/v) of the alcohol, 5-40% of the alcohol, and 10⁻⁴⁰% of the alcohol. The payload may include 20-30% (v/v) of the alcohol.

In some implementations, the payload delivery solution includes 25% (v/v) of the alcohol. Alternatively, the payload can include 2-8% (v/v) of the alcohol, or 2% of the alcohol. The alcohol may include ethanol and the payload comprises 5, 10, 20, 25, 30, and up to 40% or 50% (v/v) of ethanol, e.g., 27%. Example methods may include methanol as the alcohol, and the payload may include 5, 10, 20, 25, 30, or 40% (v/v) of the methanol. The payload may include 2-45% (v/v) of methanol, 20-30% (v/v), or 25% (v/v) methanol. Preferably, the payload includes 20-30% (v/v) of methanol. Further alternatively, the alcohol is butanol and the payload comprises 2, 4, or 8% (v/v) of the butanol.

In some aspects of the present subject matter, the payload is in an isotonic solution or buffer.

According to the present subject matter, the payload may include at least one salt. The salt may be selected from NaCl, KCl, Na₂HPO₄, C₂H₃O₂NH₄ and KH₂PO₄. For example, KCl concentration ranges from 2 mM to 500 mM. In some preferred embodiments, the concentration is greater than 100 mM, e.g., 106 mM.

According to example methods of the present subject matter, the payload may include a sugar (e.g., a sucrose, or a disaccharide). According to example methods, the payload comprises less than 121 mM sugar, 6-91 mM, or 26-39 mM sugar. Still further, the payload includes 32 mM sugar (e.g., sucrose). Optionally, the sugar is sucrose and the payload comprises 6.4, 12.8, 19.2, 25.6, 32, 64, 76.8, or 89.6 mM sucrose.

According to example methods of the present subject matter, the payload may include a buffering agent (e.g. a weak acid or a weak base). The buffering agent may include a zwitterion. According to example methods, the buffering agent is 4-(2-hydroxyethyl)-1-piperazineethanesulfonic acid. The payload may comprise less than 19 mM buffering agent (e.g., 1-15 mM, or 4-6 mM or 5 mM buffering agent). According to example methods, the buffering agent is 4-(2-hydroxyethyl)-1-piperazineethanesulfonic acid and the payload comprises 1, 2, 3, 4, 5, 10, 12, 14 mM 4-(2-hydroxyethyl)-1-piperazineethanesulfonic acid. Further preferably, the payload comprises 5 mM 4-(2-hydroxyethyl)-1-piperazineethanesulfonic acid.

According to example methods of the present subject matter, the payload includes ammonium acetate. The payload may include less than 46 mM ammonium acetate (e.g., between 2-35 mM, 10⁻¹⁵ mM, ore 12 mM ammonium acetate). The payload may include 2.4, 4.8, 7.2, 9.6, 12, 24, 28.8, or 33.6 mM ammonium acetate.

The volume of aqueous solution performed by gas propelling the aqueous solution may include compressed air (e.g. ambient air), other implementations may include inert gases, for example, helium, neon, and argon.

In certain aspects of the present subject matter, the population of cells may include adherent cells (e.g., lung, kidney, immune cells such as macrophages) or non-adherent cells (e.g., suspension cells).

In certain aspects of the present subject matter, the population of cells may be substantially confluent, and substantially may include greater than 75 percent confluent. In preferred implementations, the population of cells may form a single monolayer.

According to example methods, the payload to be delivered has an average molecular weight of up to 20,000,000 Da. In some examples, the payload to be delivered can have an average molecular weight of up to 2,000,000 Da. In some implementations, the payload to be delivered may have an average molecular weight of up to 150,000 Da. In further implementations, the payload to be delivered has an average molecular weight of up to 15,000 Da, 5,000 Da or 1,000 Da.

The payload to be delivered across the plasma membrane of a cell may include a small chemical molecule, a peptide or protein, a polysaccharide or a nucleic acid or a nanoparticle. A small chemical molecule may be less than 1,000 Da, peptides may have molecular weights about 5,000 Da, siRNA may have molecular weights around 15,000 Da, antibodies may have molecular weights of about 150,000 Da and DNA may have molecular weights of greater than or equal to 5,000,000 Da. In preferred embodiments, the payload comprises mRNA.

According to example methods, the payload includes 3.0-150.0 μM of a molecule to be delivered, more preferably, 6.6-150.0 μM molecule to be delivered (e.g. 3.0, 3.3, 6.6, or 150.0 μM molecule to be delivered). In some implementations, the payload to be delivered has an average molecular weight of up to 15,000 Da, and the payload includes 3.3 μM molecules to be delivered.

According to example methods, the payload to be delivered has an average molecular weight of up to 15,000 Da, and the payload includes 6.6 μM to be delivered. In some implementations, the payload to be delivered has an average molecular weight of up to 1,000 Da, and the payload includes 150.0 μM to be delivered.

According to further aspects of the present subject matter, a method for delivering molecules of more than one molecular weight across a plasma membrane is provided; the method including the steps of: introducing the molecules of more than one molecular weight to an aqueous solution; and contacting the aqueous solution with a plasma membrane.

In some implementations, the method includes introducing a first molecule having a first molecular weight and a second molecule having a second molecular weight to the payload, wherein the first and second molecules may have different molecular weights, or wherein, the first and second molecules may have the same molecular weights. According to example methods, the first and second molecules may be different molecules.

In some implementations, the payload to be delivered may include a therapeutic agent, or a diagnostic agent, including, for example, cisplatin, aspirin, various statins (e.g., pitavastatin, atorvastatin, lovastatin, pravastatin, rosuvastatin, simvastatin, promazine HCl, chloropromazine HCl, thioridazine HCl, Polymyxin B sulfate, chloroxine, benfluorex HCl and phenazopyridine HCl), and fluoxetine. Other therapeutic agents include antimicrobials (aminoclyclosides (e.g. gentamicin, neomycin, streptomycin), penicillins (e.g., amoxicillin, ampicillin), glycopeptides (e.g., avoparcin, vancomycin), macrolides (e.g., erythromycin, tilmicosin, tylosin), quinolones (e.g., sarafloxacin, enrofloxin), streptogramins (e.g., viginiamycin, quinupristin-dalfoprisitin), carbapenems, lipopeptides, oxazolidinones, cycloserine, ethambutol, ethionamide, isoniazrid, para-aminosalicyclic acid, and pyrazinamide). In some examples, an anti-viral (e.g., Abacavir, Aciclovir, Enfuvirtide, Entecavir, Nelfinavir, Nevirapine, Nexavir, Oseltamivir Raltegravir, Ritonavir, Stavudine, and Valaciclovir). The therapeutic may include a protein-based therapy for the treatment of various diseases, e.g., cancer, infectious diseases, hemophilia, anemia, multiple sclerosis, and hepatitis B or C.

Additional exemplary payloads can also include detectable markers or labels such as methylene blue, Patent blue V, and Indocyanine green.

The methods described herein may also include the payload including of a detectable moiety, or a detectable nanoparticle (e.g., a quantum dot). The detectable moiety may include a fluorescent molecule or a radioactive agent (e.g., ¹²⁵I). When the fluorescent molecule is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, p-phthaldehyde and fluorescamine. The molecule can also be detectably labeled using fluorescence emitting metals such as ¹⁵²Eu, or others of the lanthanide series. These metals can be attached to the molecule using such metal chelating groups as diethylenetriaminepentacetic acid (DTPA) or ethylenediaminetetraacetic acid (EDTA). The molecule also can be detectably labeled by coupling it to a chemiluminescent compound. The presence of the chemiluminescent-tagged molecule is then determined by detecting the presence of luminescence that arises during the course of chemical reaction. Examples of particularly useful chemiluminescent labeling compounds are luminol, isoluminol, theromatic acridinium ester, imidazole, acridinium salt and oxalate ester.

In additional embodiments, the payload to be delivered may include a composition that edits genomic DNA (i.e., gene editing tools). For example, the gene editing composition may include a compound or complex that cleaves, nicks, splices, rearranges, translocates, recombines, or otherwise alters genomic DNA. Alternatively or in addition, a gene editing composition may include a compound that (i) may be included a gene-editing complex that cleaves, nicks, splices, rearranges, translocates, recombines, or otherwise alters genomic DNA; or (ii) may be processed or altered to be a compound that is included in a gene-editing complex that cleaves, nicks, splices, rearranges, translocates, recombines, or otherwise alters genomic DNA. In various embodiments, the gene editing composition comprises one or more of (a) gene editing protein; (b) RNA molecule; and/or (c) ribonucleoprotein (RNP).

In some embodiments, the gene editing composition comprises a gene editing protein, and the gene editing protein is a zinc finger nuclease (ZFN), a transcription activator-like effector nuclease (TALEN), a Cas protein, a Cre recombinase, a Hin recombinase, or a Flp recombinase. In additional embodiments, the gene editing protein may be a fusion proteins that combine homing endonucleases with the modular DNA binding domains of TALENs (megaTAL). For example, megaTAL may be delivered as a protein or alternatively, a mRNA encoding a megaTAL protein is delivered to the cells.

In various embodiments, the gene editing composition comprises a RNA molecule, and the RNA molecule comprises a sgRNA, a crRNA, and/or a tracrRNA.

In certain embodiments, the gene editing composition comprises a RNP, and the RNP comprises a Cas protein and a sgRNA or a crRNA and a tracrRNA. Aspects of the present subject matter are particularly useful for controlling when and for how long a particular gene-editing compound is present in a cell.

In various implementations of the present subject matter, the gene editing composition is detectable in a population of cells, or the progeny thereof, for (a) about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution, or (b) less than about 0.5, 1, 2, 3, 4, 5, 6, 7, 8, 9, 10, 12, 24, 48, 60, 72, 0.5-2, 0.5-6, 6-12 or 0.5-72 hours after the population of cells is contacted with the aqueous solution.

In some embodiments, the genome of cells in the population of cells, or the progeny thereof, comprises at least one site-specific recombination site for the Cre recombinase, Hin recombinase, or Flp recombinase.

Aspects of the present invention relate to cells that comprise one gene editing compound, and inserting another gene editing compound into the cells. For example, one component of an RNP could be introduced into cells that express or otherwise already contain another component of the RNP. For example, cells in a population of cells, or the progeny thereof, may comprise a sgRNA, a crRNA, and/or a tracrRNA. In some embodiments the population of cells, or the progeny thereof, expresses the sgRNA, crRNA, and/or tracrRNA. Alternatively or in addition, cells in a population of cells, or the progeny thereof, express a Cas protein.

Various implementations of the subject matter herein include a Cas protein. In some embodiments, the Cas protein is a Cas9 protein or a mutant thereof. Exemplary Cas proteins (including Cas9 and non-limiting examples of Cas9 mutants) are described herein.

In various aspects, the concentration of Cas9 protein may range from about 0.1 to about 25 μg. For example, the concentration of Cas9 may be about 1 μg, about 5 μg, about 10 μg, about 15 μg, or about 20 μg. Alternatively, the concentration of Cas9 may range from about 10 ng/μL to about 300 ng/μL; for example from about 10 ng/μL to about 200 ng/μl; or from about 10 ng/μL to about 100 ng/μl, or from about 10 ng/μL to about 50 ng/μl.

In certain embodiments, the gene editing composition comprises (a) a first sgRNA molecule and a second sgRNA molecule, wherein the nucleic acid sequence of the first sgRNA molecule is different from the nucleic acid sequence of the second sgRNA molecule; (b) a first RNP comprising a first sgRNA and a second RNP comprising a second sgRNA, wherein the nucleic acid sequence of the first sgRNA molecule is different from the nucleic acid sequence of the second sgRNA molecule; (c) a first crRNA molecule and a second crRNA molecule, wherein the nucleic acid sequence of the first crRNA molecule is different from the nucleic acid sequence of the second crRNA molecule; (d) a first crRNA molecule and a second crRNA molecule, wherein the nucleic acid sequence of the first crRNA molecule is different from the nucleic acid sequence of the second crRNA molecule, and further comprising a tracrRNA molecule; or (e) a first RNP comprising a first crRNA and a tracrRNA and a second RNP comprising a second crRNA and a tracrRNA, wherein the nucleic acid sequence of the first crRNA molecule is different from the nucleic acid sequence of the second crRNA molecule.

In aspects, the ratio of the Cas9 protein to guide RNA may be 1:1, 1:2, 1:3, 1:4, 1:5, 1:6, 1:7, 1:8, 1:9, or 1:10.

In embodiments, increasing the number of times that cells go through the delivery process (alternatively, increasing the number of doses), may increase the percentage edit; wherein, in some embodiments the number of doses may include 1, 2, 3, 4, 5, 6, 7, 8, 9, or 10 doses.

In various embodiments, the first and second sgRNA or first and second crRNA molecules together comprise nucleic acid sequences complementary to target sequences flanking a gene, an exon, an intron, an extrachromosomal sequence, or a genomic nucleic acid sequence, wherein the gene, an exon, intron, extrachromosomal sequence, or genomic nucleic acid sequence is about 1, 2, 3, 4, 5, 6, 10, 20, 30, 40, 50, 60, 70, 80, 90, 100, 1-100, kilobases in length or is at least about 1, 2, 3, 4, 5, 6, 10, 20, 30, 40, 50, 60, 70, 80, 90, 100, 1-100, kilobases in length. In some embodiments, the use of pairs of RNPs comprising the first and second sgRNA or first and second crRNA molecules may be used to create a polynucleotide molecule comprising the gene, exon, intron, extrachromosomal sequence, or genomic nucleic acid sequence.

In certain embodiments, the target sequence of a sgRNA or crRNA is about 12 to about 25, or about 12, 13, 14, 15, 16, 17, 18, 19, 20, 21, 22, 23, 24, 25, 17-23, or 18-22, nucleotides long. In some embodiments, the target sequence is 20 nucleotides long or about 20 nucleotides long.

In various embodiments, the first and second sgRNA or first and second crRNA molecules are complementary to sequences flanking an extrachromosomal sequence that is within an expression vector.

Aspects of the present subject matter relate to the delivery of multiple components of a gene-editing complex, where the multiple components are not complexed together. In some embodiments, gene editing composition comprises at least one gene editing protein and at least one nucleic acid, wherein the gene editing protein and the nucleic acid are not bound to or complexed with each other.

The present subject matter allows for high gene editing efficiency while maintaining high cell viability. In some embodiments, at least about 10, 20, 30, 40, 50, 60, 70, 80, 90, 95, 99%, 1-99%, or more of the population of cells, or the progeny thereof, become genetically modified after contact with the aqueous solution. In various embodiments, at least about 10, 20, 30, 40, 50, 60, 70, 80, 90, 95, 99%, 1-99%, or more of the population of cells, or the progeny thereof, are viable after contact with the aqueous solution.

In certain embodiments, the gene editing composition induces single-strand or double-strand breaks in DNA within the cells. In some embodiments the gene editing composition further comprises a repair template polynucleotide. In various embodiments, the repair template comprises (a) a first flanking region comprising nucleotides in a sequence complementary to about 40 to about 90 base pairs on one side of the single or double strand break and a second flanking region comprising nucleotides in a sequence complementary to about 40 to about 90 base pairs on the other side of the single or double strand break; or (b) a first flanking region comprising nucleotides in a sequence complementary to at least about 20, 25, 30, 35, 40, 45, 50, 60, 70, 80, or 90 base pairs on one side of the single or double strand break and a second flanking region comprising nucleotides in a sequence complementary to at least about 20, 25, 30, 35, 40, 45, 50, 60, 70, 80, or 90 base pairs on the other side of the single or double strand break. Non-limiting descriptions relating to gene editing (including repair templates) using the CRISPR-Cas system are discussed in Ran et al. (2013) Nat Protoc. 2013 November; 8(11): 2281-2308, the entire content of which is incorporated herein by reference. Embodiments involving repair templates are not limited to those comprising the CRISPR-Cas system.

In various implementations of the present subject matter, the volume of aqueous solution is delivered to the population of cells in the form of a spray. In some embodiments, the volume is between 6.0×10⁻⁷ microliter per cell and 7.4×10⁴ microliter per cell. In certain embodiments, the spray comprises a colloidal or sub-particle comprising a diameter of 10 nm to 100 μm. In various embodiments, the volume is between 2.6×10⁻⁹ microliter per square micrometer of exposed surface area and 1.1×10⁻⁶ microliter per square micrometer of exposed surface area.

In some embodiments, the RNP has a size of approximately 100 Å×100 Å×50 Å or 10 nm×10 nm×5 nm. In various embodiments, the size of spray particles is adjusted to accommodate at least about 1, 2, 3, 4, 5, 6, 7, 8, 9, 10, or more RNPs per spray particle.

For example, contacting the population of cells with the volume of aqueous solution may be performed by gas propelling the aqueous solution to form a spray. In certain embodiments, the population of cells is in contact with said aqueous solution for 0.01-10 minutes (e.g., 0.1 10 minutes) prior to adding a second volume of buffer or culture medium to submerse or suspend said population of cells.

In various embodiments, the population of cells includes at least one of primary or immortalized cells. For example, the population of cells may include mesenchymal stem cells, lung cells, neuronal cells, fibroblasts, human umbilical vein (HUVEC) cells, and human embryonic kidney (HEK) cells, primary or immortalized hematopoietic stem cell (HSC), T cells, natural killer (NK) cells, cytokine-induced killer (CIK) cells, human cord blood CD34+ cells, B cells. Non limiting examples of T cells may include CD8+ or CD4+ T cells. In some aspects, the CD8+ subpopulation of the CD3+ T cells are used. CD8+ T cells may be purified from the PBMC population by positive isolation using anti-CD8 beads. In some aspects primary NK cells are isolated from PBMCs and GFP mRNA may be delivered by platform delivery technology (i.e., 3% expression and 96% viability at 24 hours). In additional aspects, NK cell lines, e.g., NK92 may be used.

Cell types also include cells that have previously been modified for example T cells, NK cells and MSC to enhance their therapeutic efficacy, and use for 3-dimensional cultures, tissue explants, skin grafts, engineered tissues, and the like. For example: T cells or NK cells that express chimeric antigen receptors (CAR T cells, CAR NK cells, respectively); T cells that express modified T cell receptor (TCR); MSC that are modified virally or non-virally to overexpress therapeutic proteins that complement their innate properties (e.g. delivery of Epo using lentiviral vectors or BMP-2 using AAV-6) (reviewed in Park et al, Methods, 2015 August; 84-16); MSC that are primed with non-peptidic drugs or magnetic nanoparticles for enhanced efficacy and externally regulated targeting respectively (Park et al., 2015); MSC that are functionalised with targeting moieties to augment their homing toward therapeutic sites using enzymatic modification (e.g. Fucosyltransferase), chemical conjugation (eg. modification of SLeX on MSC by using N-hydroxy-succinimide (NHS) chemistry) or non-covalent interactions (eg. engineering the cell surface with palmitated proteins which act as hydrophobic anchors for subsequent conjugation of antibodies) (Park et al., 2015). For example, T cells, e.g., primary T cells or T cell lines, that have been modified to express chimeric antigen receptors (CAR T cells) may further be treated according to the invention with gene editing proteins and or complexes containing guide nucleic acids specific for the CAR encoding sequences for the purpose of editing the gene(s) encoding the CAR, thereby reducing or stopping the expression of the CAR in the modified T cells.

Aspects of the present invention relate to the expression vector-free delivery of gene editing compounds and complexes to cells and tissues, such as delivery of Cas-gRNA ribonucleoproteins for genome editing in primary human T cells, hematopoietic stem cells (HSC), and mesenchymal stromal cells (MSC). In some example, mRNA encoding such proteins are delivered to the cells.

Various aspects of the CRISPR-Cas system are known in the art. Non-limiting aspects of this system are described, e.g., in U.S. Pat. No. 9,023,649, issued May 5, 2015; U.S. Pat. No. 9,074,199, issued Jul. 7, 2015; U.S. Pat. No. 8,697,359, issued Apr. 15, 2014; U.S. Pat. No. 8,932,814, issued Jan. 13, 2015; PCT International Patent Application Publication No. WO 2015/071474, published Aug. 27, 2015; Cho et al., (2013) Nature Biotechnology Vol 31 No 3 pp 230-232 (including supplementary information); and Jinek et al., (2012) Science Vol 337 No 6096 pp 816-821, the entire contents of each of which are incorporated herein by reference.

Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmrl, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2 and in the NCBI database as under accession number Q99ZW2.1. UniProt database accession numbers AOAOG4DEU5 and CDJ55032 provide another example of a Cas9 protein amino acid sequence. Another non-limiting example is a Streptococcus thermophilus Cas9 protein, the amino acid sequence of which may be found in the UniProt database under accession number Q03JI6.1. In some embodiments, the unmodified CRISPR enzyme has DNA cleavage activity, such as Cas9. In certain embodiments the CRISPR enzyme is Cas9, and may be Cas9 from S. pyogenes or S. pneumoniae. In various embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution in the RuvC I catalytic domain of Cas9 from S. pyogenes converts Cas9 from a nuclease that cleaves both strands to a nickase (cleaves a single strand). Other examples of mutations that render Cas9 a nickase include, without limitation, H840A, N854A, and N863A. In aspects of the invention, nickases may be used for genome editing via homologous recombination.

In certain embodiments, a Cas9 nickase may be used in combination with guide sequence(s), e.g., two guide sequences, which target respectively sense and antisense strands of the DNA target. This combination allows both strands to be nicked and used to induce NHEJ.

As a further example, two or more catalytic domains of Cas9 (RuvC I, RuvC II, and RuvC III) may be mutated to produce a mutated Cas9 substantially lacking all DNA cleavage activity. A D10A mutation may be combined with one or more of H840A, N854A, or N863A mutations to produce a Cas9 enzyme substantially lacking all DNA cleavage activity. In certain embodiments, a CRISPR enzyme is considered to substantially lack all DNA cleavage activity when the DNA cleavage activity of the mutated enzyme is less than about 25%, 10%, 5%, 1%, 0.1%, 0.01%, or lower with respect to its non-mutated form. Other mutations may be useful; where the Cas9 or other CRISPR enzyme is from a species other than S. pyogenes, mutations in corresponding amino acids may be made to achieve similar effects.

In certain embodiments, a protein being delivered (such as a Cas protein or a variant thereof) may include a subcellular localization signal. For example, the Cas protein within a RNP may comprise a subcellular localization signal. Depending on context, a fusion protein comprising, e.g., Cas9 and a nuclear localization signal may be referred to as “Cas9” herein without specifying the inclusion of the nuclear localization signal. In some embodiments, the payload (such as an RNP) comprises a fusion-protein that comprises a localization signal. For example, the fusion-protein may contain a nuclear localization signal, a nucleolar localization signal, or a mitochondrial targeting signal. Such signals are known in the art, and non-limiting examples are described in Kalderon et al., (1984) Cell 39 (3 Pt 2): 499-509; Makkerh et al., (1996) Curr Biol. 6 (8):1025-7; Dingwall et al., (1991) Trends in Biochemical Sciences 16 (12): 478-81; Scott et al., (2011) BMC Bioinformatics 12:317 (7 pages); Omura T (1998) J Biochem. 123(6):1010-6; Rapaport D (2003) EMBO Rep. 4(10):948-52; and Brocard & Hartig (2006) Biochimica et Biophysica Acta (BBA)—Molecular Cell Research 1763(12):1565-1573, the contents of each of which are hereby incorporated herein by reference. In various embodiments, the Cas protein may comprise more than one localization signals, such as 2, 3, 4, 5, or more nuclear localization signals. In some embodiments, the localization signal is at the N-terminal end of the Cas protein and in other embodiments the localization signal is at the C-terminal end of the Cas protein.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis.

Accordingly, genes can be tailored for optimal gene expression in a given organism based on codon optimization. Codon usage tables are readily available, for example, at the “Codon Usage Database”, and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding a CRISPR enzyme corresponding to the most frequently used codon for a particular amino acid.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some embodiments, the degree of complementarity is 100%. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In certain embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions.

CRISPR-Cas technology which facilitates genome engineering in a wide range of cell types is evolving rapidly. It has recently been shown that delivery of the Cas9-gRNA editing tools in the form of ribonucleoproteins (RNPs) yields several benefits compared with delivery of plasmids encoding for Cas9 and gRNAs. Benefits include faster and more efficient editing, fewer off-target effects, and less toxicity. RNPs have been delivered by lipofection and electroporation but limitations that remain with these delivery methods, particularly for certain clinically relevant cell types, include toxicity and low efficiency. Accordingly, there is a need to provide a vector-free e.g., viral vector-free, approach for delivering biologically relevant payloads, e.g., RNPs, across a plasma membrane and into cells. “Cargo” or “payload” are terms used to describe a compound, or composition that is delivered via an aqueous solution across a cell plasma membrane and into the interior of a cell.

The current subject matter relates to delivery technology that facilitates delivery of a broad range of payloads to cells with low toxicity. Genome editing may be achieved by delivering RNPs to cells using some aspects of the current subject matter. Levels decline thereafter until Cas9 is no longer detectable. The delivery technology per se does not deleteriously affect the viability or functionality of Jurkat and primary T cells. The current subject matter enables gene editing via Cas9 RNPs in clinically relevant cell types with minimal toxicity.

The transient and direct delivery of CRISPR/Cas components such as Cas and/or a gRNA has advantages compared to expression vector-mediated delivery. For example, an amount of Cas, gRNA, or RNP can be added with more precise timing and for a limited amount of time compared to the use of an expression vector. Components expressed from a vector may be produced in various quantities and for variable amounts of time, making it difficult to achieve consistent gene editing without off-target edits. Additionally, pre-formed complexes of Cas and gRNAs (RNPs) cannot be delivered with expression vectors.

In one aspect, the present subject matter describes cells attached to a solid support, (e.g., a strip, a polymer, a bead, or a nanoparticle). The support or scaffold may be a porous or non-porous solid support. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present subject matter. The support material may have virtually any possible structural configuration. Thus, the support configuration may be spherical, as in a bead, or cylindrical, as in the inside surface of a test tube, or the external surface of a rod. Alternatively, the surface may be flat such as a sheet, or test strip, etc. Preferred supports include polystyrene beads.

In other aspects, the solid support comprises a polymer, to which cells are chemically bound, immobilized, dispersed, or associated. A polymer support may be a network of polymers, and may be prepared in bead form (e.g., by suspension polymerization). The cells on such a scaffold can be sprayed with payload containing aqueous solution according to the invention to deliver desired compounds to the cytoplasm of the scaffold. Exemplary scaffolds include stents and other implantable medical devices or structures.

### Example 4

Effect of alcohol on RNP (ribonucleoprotein)—edit efficiency post-delivery by the example delivery platforms illustrated in FIGS. 1 and 23.

Experiments were performed to determine the effect alcohol (e.g., ethanol) had on RNP-edit efficiency post-delivery using the example delivery platforms illustrated in FIGS. 1 and 23. Additionally, the experiments were performed to ascertain an optimal ethanol concentration for editing following delivery of RNP by the example delivery platforms illustrated in FIGS. 1 and 23. For example, the maximum ethanol concentration which allowed for optimal Cas9-induced edit was determined. An increase in ethanol allowed for more cargo delivery to the cell, and thereby allowing for greater edit efficiency.

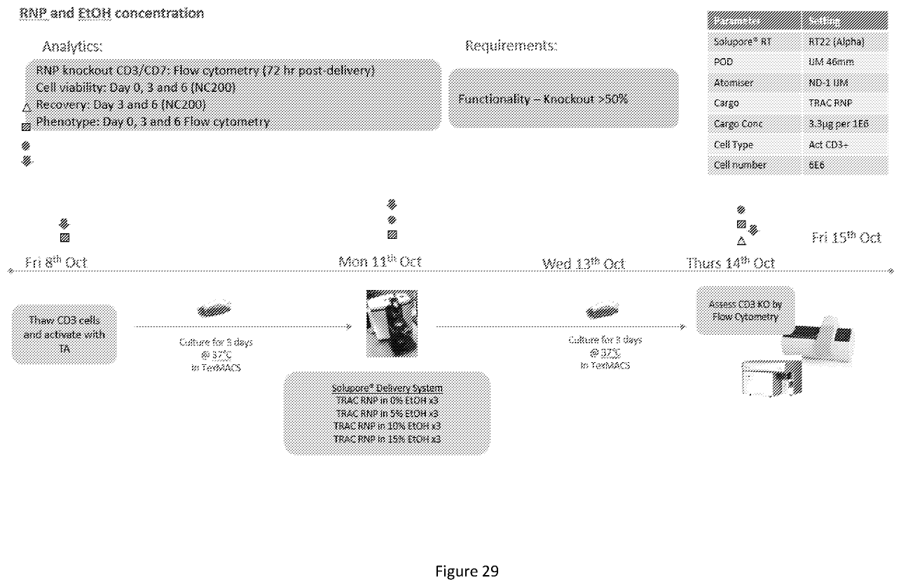

Cas9 RNP—TRAC (T cell receptor alpha constant) sgRNA (single guide RNA) was prepared at 2:1 ratio at 0.4 μg/μL (equiv to 3.3 μg per 1×10⁶ cells); S Buffer (32.5 mM sucrose; 106 mM potassium chloride; 5 mM HEPES) solutions were prepared with 0, 5, 10 and 15% ethanol with RNP and the experiments were carried out on the example delivery platforms illustrated in FIGS. 1 and 23. with the S buffer solutions at each ethanol concentration. The TRAC guide RNA sequence: AGAGTCTCTCAGCTGGTACA (SEQ ID NO: 1). In embodiments, at least two exogenous cargos are simultaneously delivered, meaning the two exogenous cargos are delivered at the same time (e.g., dual delivery). For example the immune cell (comprising an exogenous cargo), may be manipulated to comprise a second exogenous cargo. The experimental design is shown in FIG. 29.

“S Buffer” includes a hypotonic physiological buffered solution (78 mM sucrose, 30 mM KCl, 30 mM potassium acetate, 12 mM HEPES) for 5 min at 4° C. (Medepalli K. et al., Nanotechnology 2013; 24(20); incorporated herein by reference in its entirety). In some examples, potassium acetate is replaced with ammonium acetate in the S Buffer. S buffer is further described in international application WO 2016/065341, e.g., at ¶ [0228]-[0229] and incorporated herein by reference in its entirety. For example, the S buffer used in series of experiments described herein included 32.5 mM sucrose; 106 mM potassium chloride; and 5 mM HEPES.

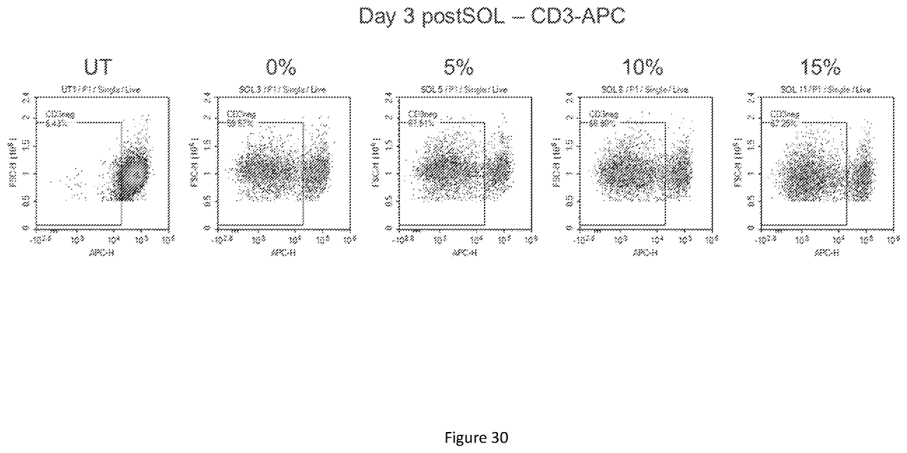

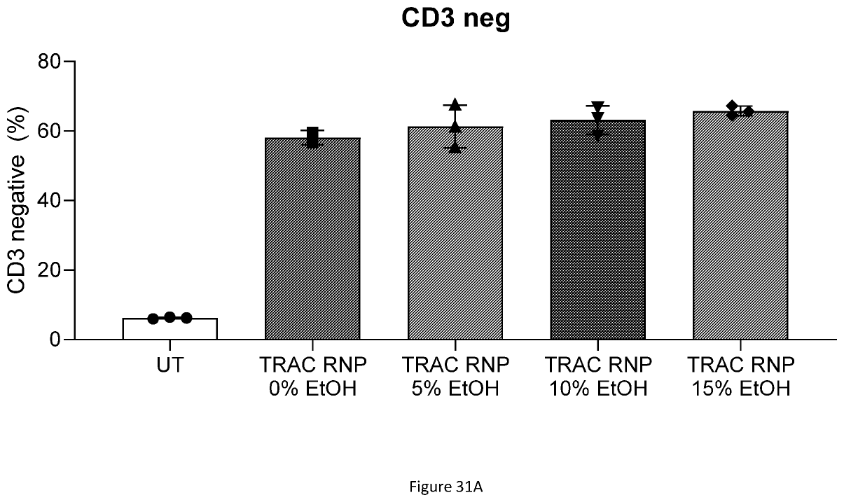

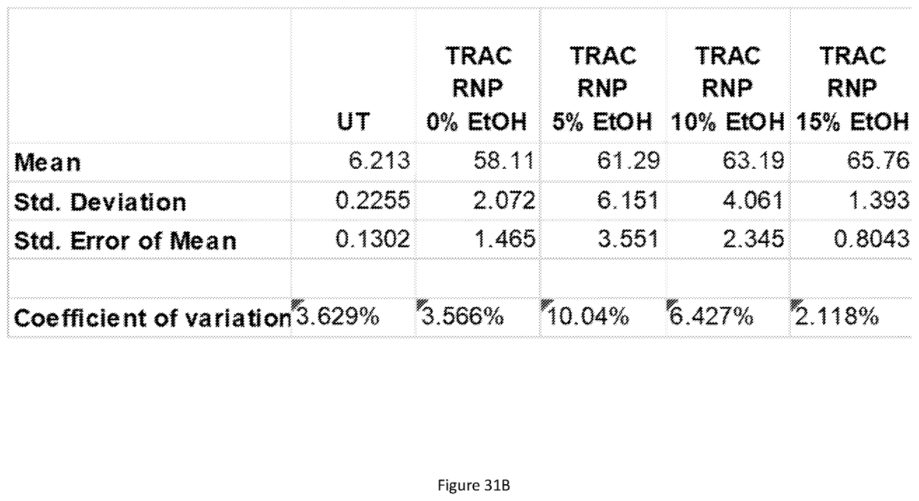

Conclusion: CD3 (cluster of differentiation 3) edit efficiencies (e.g., monitoring TRAC RNP) at each ethanol concentration was tested post-delivery using the example delivery platforms illustrated in FIGS. 1 and 23. See FIG. 30 depicting representative flow cytometry plots from cells stained with an antibody targeting CD3 (gated off the live population) and FIG. 31.

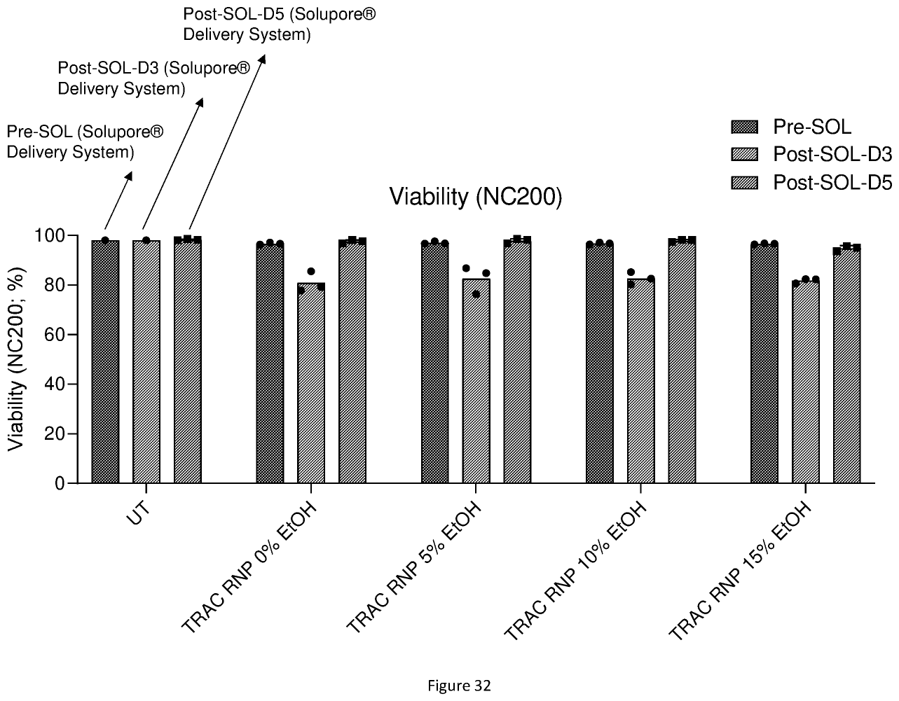

FIG. 31A shows a bar graph showing that the level of CD3 edit increased modestly with increasing concentrations of ethanol (0% EtOH and 58% CD3 edit to 15% EtOH and 66% CD3 edit), and the results are further summarized in the table in FIG. 31B. The percent viability at the increasing ethanol concentrations, and time points consisting of pre-delivery, post-delivery (day 3) and post-delivery (day 5) are summarized in the bar graph in FIG. 32.

### Example 5

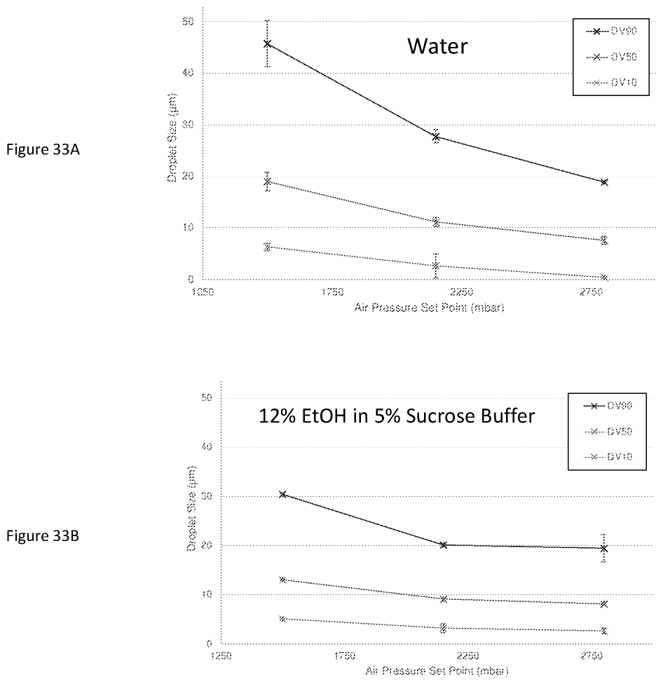

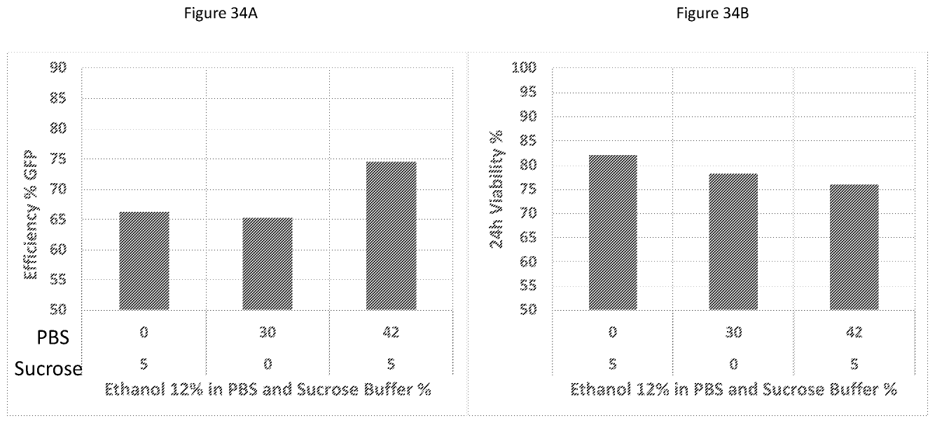

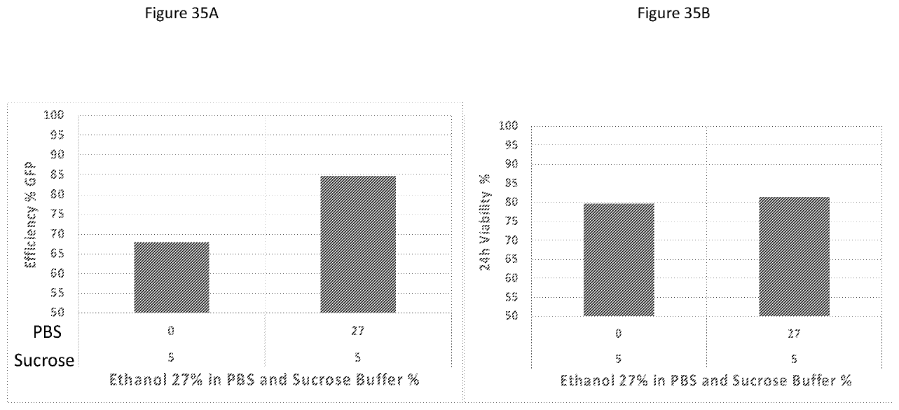

FIGS. 33A and 33B illustrate droplet size versus pressure of atomization for the example delivery platform when employing a 0% EtOH delivery solution (FIG. 33A, no alcohol) and with 12.5% alcohol (FIG. 33B). In FIGS. 33A and 33B, DV90 indicates that the portion of particles with diameters smaller than this value is 90%, DV50 indicates that the portion of particles with diameters smaller than this value is 50%, and DV10 indicates that the portion of particles with diameters smaller than this value is 10%.

In some implementations, aqueous solutions without ethanol showed a larger droplet size (for a given pressure for atomizing the solution), which required additional consideration of process conditions to give optimal spay coverage of cells with cargo for transfection.

In some implementations, when the platform is utilizing a 0% ethanol delivery solution, additional wash steps can be omitted. The on/off switching speed of the spray delivery can remain constant. Similarly, the plume and nozzle design can used for ethanol or no ethanol solutions. As described in more detail in Example 6, the system can also provide for delivery using a hypertonic solution (e.g., a much higher salt concentration in the delivery solution).

For cells of approximately 10 μm in diameter (e.g. human T cells) FIGS. 33A and 33B are line graphs showing that the spray droplet size required higher atomisation pressures to be applied to maintain the droplet size range closer to cell size, including to avoid excessively large droplets. Droplet size was measured as D90 using Malvern Mastersizer 3000 laser diffraction apparatus (available from Malvern Panalytical Ltd., Malvern, United Kingdom; see, (<https://www.malvernpanalytical.com/en/products/product-range/mastersizer-range/mastersizer-3000>). In some implementations, the example delivery platform can utilize a pressure where a distribution of spray droplet (e.g., particle) size distribution can include a size range where D90 is not more than 5 times cell size, a range where D90 is not more than 3.3× cell size, and/or a range where D90 is not larger than about 2× cell size.

### Example 6

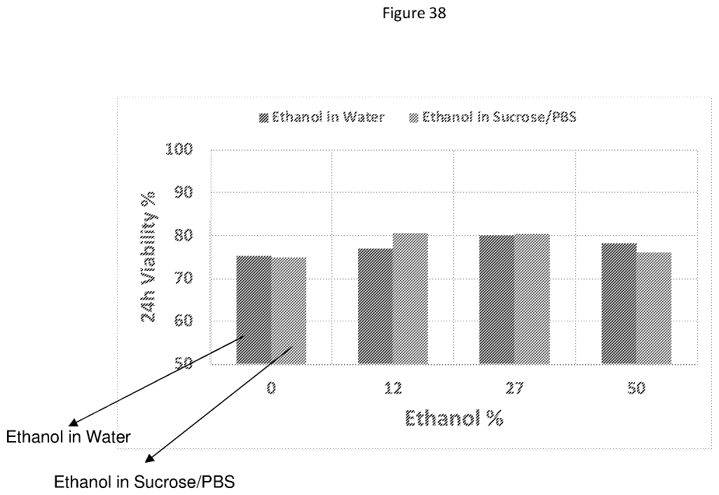

Effect of a hypertonic delivery system as illustrated in FIG. 34 and FIG. 34B, FIGS. 35A and 35B, FIGS. 36-38.

Increasing delivery solution osmolality (e.g., the effect of a hypertonic solution) was studied using various ethanol concentrations, including 0% EtOH, in delivery solutions of various volume ratios of Sucrose buffer (45% sucrose ca. 175 mOsm/kg) and phosphate buffered saline (PBS) (ca. 300 mOsm/kg), shown in the table below.

TABLE 1
Increasing delivery solution osmolality
at various ethanol concentrations

| | | Concentration |
|---|---|---|
| Component | Molecular Weight | (mg/L) |
| Potassium Chloride | 75.0 | 200.0 |
| Sodium Phosphate monobasic | 136.0 | 200.0 |
| Sodium Chloride | 58.0 | 8000.0 |
| Potassium Phosphate dibasic | 268.0 | 2160.0 |

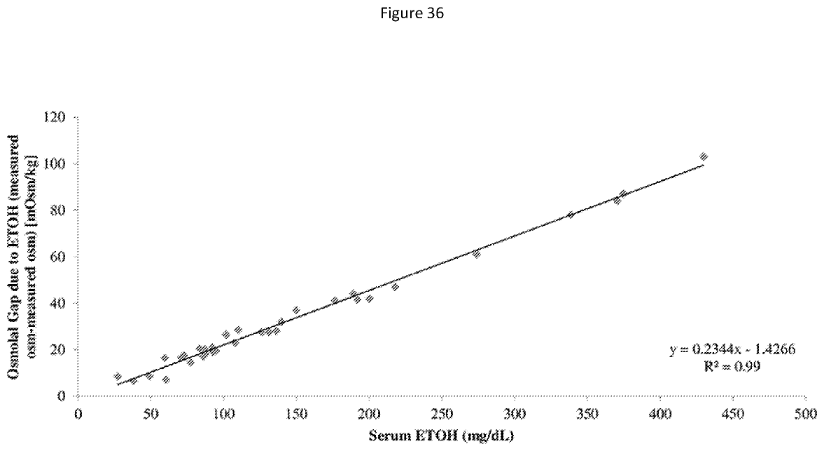

Results: FIGS. 34A and 34B, as well as FIGS. 35A and 35B showed that increased in GFP transfection achieved using 12% and 27% ethanol in solutions increasing the proportions of sucrose and sodium chloride from the two buffer solutions. In FIGS. 34A, 34B, as well as 35 Å and 35B, the cell viability was also maintained. Ethanol had a higher impact on the osmolality, as demonstrated by measuring the effect of ethanol in serum (see, e.g., Nguyen, M. et al “Front. Med. Is the Osmolal Concentration of Ethanol Greater Than Its Molar Concentration? Jan. 8, 2020, “Nguyen” incorporated herein by reference in its entirety). FIG. 1 from the Nguyen reference is reproduced herein (FIG. 36) illustrating a linear regression analysis relating the osmolality gap solely due to ethanol based on the difference between measured serum osmolality after ethanol addition and measured serum osmolality before ethanol addition and serum ethanol concentration in mg/dL.

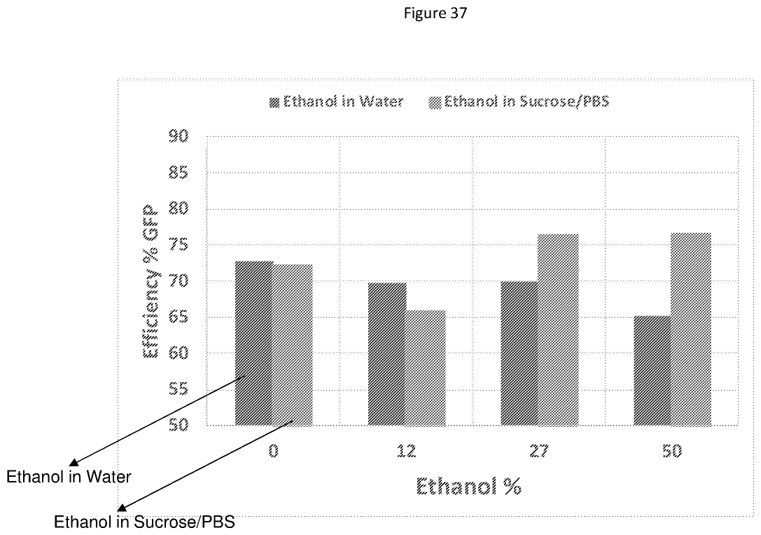

The hypertonic solutions described and studied herein also increased transfection (FIG. 37).

Hypertonic solutions can increase transfection, and also can decrease viability. Hypertonic solutions preferably contain both organic components such as sucrose or other pharmaceutically acceptable saccharides like dextrose, glucose, sorbitol, mannitol, and inorganic salts such as sodium chloride, potassium chloride or other pharmaceutically acceptable salts. The combined delivery solution, without ethanol can use osmolality less than 300 mosm/kg, or more than 300 mosm/kg such as up to 400 mosm/kg or up to 500 mosm/kg. This solution can then be mixed with ethanol in varying amounts up to 50%. The relative amounts of saccharide and inorganic salts may vary such that up to 50% of the osmolality of the aqueous buffer mixture arises from the saccharides (or combination with inorganic salts thereof), with preferred ranges being less than 40% and most preferred less than 33% as saccharide. In other examples, up to 40%, up to 35%, up to 34%, up to 33%, up to 32%, up to 31% or up to 30% of the osmolality of the aqueous buffer mixture arises from the saccharides (or combination with inorganic salts thereof).

In some embodiments, the delivery solution is hypertonic, wherein the osmolality of the solution is affected by the combination of a saccharide (e.g., sucrose, dextrose, glucose, sorbitol, mannitol, and other pharmaceutically acceptable saccharides) and an inorganic salt (e.g., sodium chloride, potassium chloride or other pharmaceutically acceptable salts). In some examples, the delivery solution can include a mixture of more than one saccharide and a mixture of more than one inorganic salt.

As much as 50% of the osmolality of the aqueous buffer can arise from a mixture of saccharides. For example, the preferred ranges include less than 40%, or more preferably less than 33% of the osmolality arises from the saccharide (e.g., the osmolality of the aqueous solution is rendered by the saccharides, or mixtures of saccharides thereof).

In other embodiments, the delivery solution (without alcohol, but including at least one saccharide and inorganic salt) can have an osmolality of less than 300 mOsm/kg, equal to or about 300 mOsm/kg, up to 400 mOsm/kg, or up to 500 mOsm/kg. In other examples the delivery solution (without alcohol, but including at least one saccharide and one organic salt) can have an osmolality of about 300 mOsm/kg, or about 350 mOsm/kg, or about 400 mOsm/kg, or about 450 mOsm/kg, or about 500 mOsm/kg. In other examples the delivery solution (without alcohol, but including at least one saccharide and one organic salt) can have an osmolality of about 320 mOsm/kg, about 330 mOsm/kg, about 340 mOsm/kg, about 350 mOsm/kg, about 360 mOsm/kg, about 370 mOsm/kg, about 380 mOsm/kg, about 390 mOsm/kg, about 400 mOsm/kg, about 410 mOsm/kg, about 420 mOsm/kg, about 430 mOsm/kg, about 440 mOsm/kg, about 450 mOsm/kg, about 460 mOsm/kg, about 470 mOsm/kg, about 480 mOsm/kg, about 490 mOsm/kg, or about 500 mOsm/kg.

### OTHER EMBODIMENTS

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A method comprising:
filling a pod of a cell engineering platform with a mixture of cells and a first medium; and
discharging the first medium from the pod through a filter, leaving the cells deposited on the filter,
wherein the cell engineering platform includes:
an atomizer; and
a pod holder configured to receive the pod; and
wherein the pod includes a filter plate and an upper portion forming a well for holding cells and media.

2. The method of claim 1, further comprising spraying a delivery solution that contains a payload to the cells deposited on the filter.

3. The method of claim 2, further comprising applying a stop solution in the chamber.

4. The method of claim 3, further comprising filling the pod with a second medium to resuspend the cells from the filter.

5. The method of claim 4, wherein the discharged first medium is reused as the second medium.

6. The method of claim 4, further comprising agitating the pod.

7. The method of claim 4, further comprising extracting the resuspended cells from the pod.

8. The method of claim 4, wherein the filling the pod is performed automatically with a pump and a controller.

9. The method of any one of claim 1, further comprising culturing the cells within the pod.

10. The method of claim 1, wherein the discharging the first medium from the pod is performed by supplying a vacuum to the bottom of the pod.

11. The method of claim 1, wherein the discharging the first medium from the pod is performed by gravity.

12. The method of claim 3, wherein the applying the stop solution is performed to wash the cells.

13. The method of claim 4, wherein the filling the chamber with the second medium is performed as at least one of a cell wash process, a cell concentration change process, and a cell medium change process.

14. The method of any one of claim 1, wherein the pod includes a lower portion releasably coupled to the filter plate.

15. The method of any one of claim 1, wherein the pod includes a memory storing data characterizing at least one process parameter.

16. The method of claim 15, further comprising:
reading, by a controller of the cell engineering platform, the at least one process parameter from the memory; and
performing at least one processing step utilizing the at least one processing parameter.

17. The method claim 1, wherein the pod includes a memory storing data characterizing an experiment identifier.

18. The method of claim 1, wherein the pod comprises a chamber.

19. A system comprising:
a housing including a pod holder configured to receive a pod, the pod including a filter plate and an upper portion forming a well;
a delivery solution applicator configured to deliver atomized delivery solution to the well;
a display; and
a controller including circuitry configured to display at least one process parameter.

20. The system of claim 19, wherein the pod holder is configured to tilt or vibrate the pod.

21. The system of claim 19, wherein the delivery solution applicator includes a spray head.

22. The system of claim 19, wherein the pod is sized to hold less than 1×10⁷ T cells.

23. The system of claim 19, wherein the system is configured to automatically:
apply an atomized delivery solution to a cellular monolayer formed on a filter within the pod.

24. The system of claim 19, wherein the delivery solution applicator includes a nebulizer.

25. The system of claim 24, wherein the delivery solution applicator further includes a mass flow controller or a volumetric flow controller to regulate a gas flow to operate the nebulizer.

26. The system of claim 19, wherein the delivery solution applicator is configured to deliver 10⁻³⁰⁰ micro liters of the delivery solution per actuation.

27. The system of claim 19, further comprising a reservoir containing the delivery solution, wherein the delivery solution includes an aqueous solution, the aqueous solution including a payload.

28. The system of claim 27, wherein said aqueous solution includes an alcohol at greater than 0.2 percent (v/v) concentration and said alcohol comprises ethanol.

29. The system of claim 28, wherein said aqueous solution comprises greater than 5% ethanol.

30. The system of claim 28, wherein said aqueous solution comprises between 5-30% ethanol.

31. The system of claim 27, wherein said aqueous solution is alcohol free.

32. The system of claim 27, wherein said aqueous solution comprises between 12.5-500 mM KCl.

33. The system of claim 27, wherein said aqueous solution comprises 106 mM KCl.

34. The system of claim 19, wherein the well is configured to contain a population of non-adherent cells.

35. The system of claim 34, wherein said non-adherent cell comprises a peripheral blood mononuclear cell.

36. The system of claim 34, wherein said non-adherent cell comprises an immune cell.

37. The system of claim 34, wherein said non-adherent cell comprises a T lymphocyte.

38. The system of claim 27, wherein said payload comprises a messenger ribonucleic acid (mRNA).

39. The system of claim 38, wherein said mRNA encodes a gene-editing composition.

40. The system of claim 39, wherein said gene editing composition reduces the expression of PD-1.

41. The system of claim 38, wherein said mRNA encodes a chimeric antigen receptor.

42. The system of claim 19, wherein the system is configured for use to deliver a cargo compound or composition to a mammalian cell.

43. The system of claim 34, wherein said population of non-adherent cells comprises a monolayer.

44. The system of claim 19, wherein the pod includes a memory storing data characterizing the at least one process parameter.

45. The system of claim 44, wherein the controller is further configured to
read, via the circuitry, the data characterizing the at least one process parameter from the memory, and
perform, via the circuitry, at least one processing step utilizing the at least one processing parameter.

46. The system of claim 19, wherein the pod includes a memory storing data characterizing an experiment identifier.

47. A system comprising:
a housing including
a base,
at least one controller including circuitry configured to control an operation of the system, and
a display;
one or more fluid circuits including at least one valve, at least one pump, a syringe, and at least one fluid detection sensor;
a chamber assembly received within an articulating frame extending from the front surface of the housing, wherein the chamber assembly is sealed from atmospheric conditions in operation and includes a filter;
at least one media container;
at least one cell culture container fluidically coupled to the chamber assembly via the one or more fluid circuits; and
at least one collection tray configured to receive media or cells.

48. The system of claim 47, wherein the articulating frame is configured to agitate the chamber assembly.

49. The system of claim 47, wherein the chamber assembly includes a memory storing data characterizing at least one process parameter.

50. The system of claim 49, wherein the at least one controller is configured to
read, via the circuitry, the data characterizing the at least one process parameter from the memory, and
perform, via the circuitry, at least one processing step utilizing the at least one processing parameter.

51. The system of claim 47, wherein the chamber assembly includes a memory storing data characterizing an experiment identifier.

52. The system of claim 47, wherein the operation of the system includes at least one of a cell wash process, a cell concentration change process, and a cell medium change process.

53. The system of claim 47, wherein the display includes a human-machine interface configured to receive inputs associated with the operation of the system.

54. The system of claim 47, wherein the articulating frame is configured to articulate to an angle between 0-10.0, 10.1-15.0, 15.1-20.0, 20.1-25.0, 25.1-30.0, 30.1-35.0, 35.1-40.0, or 40.1-45.0 degrees with respect to a horizontal surface on which the system is positioned.

55. The system of claim 47, wherein the articulating frame is configured to oscillate between two angles at a predetermined or user-defined frequency.

56. The system of claim 55, wherein the predetermined or user-defined frequency is between 0.5 kHz, 0.51-1.0 kHz, 1.1-1.5 kHz, 1.51-2.0 kHz, 2.01-2.5 kHz, or greater than 2.51 kHz.

57. The system of claim 47, wherein the at least one collection tray includes a cooling element or a heating element.

58. The system of claim 47, wherein the base includes a scale positioned below the at least one collection tray.

59. The system of claim 47, wherein the at least one fluid detection sensor is arranged with respect to at least one fluidic circuit of the one or more fluidic circuits.

60. The system of claim 59, wherein a first fluid detection sensor is configured at a first location of the at least one fluidic circuit and a second fluid detection sensor is configured at a second location of the at least one fluidic circuit, the first fluid detection sensor and the second fluid detection sensor operable to calculate a volume of the media between the first location and a second location of the at least one fluidic circuit.

61. The system of claim 47, wherein the at least one pump is a peristaltic pump.

62. The system of claim 47, wherein the system includes a syringe holder to hold the syringe, the syringe holder including an optical sensor configured to determine a level of fluid within the syringe or a position of a plunger of the syringe.

63. The system of claim 62, wherein the optical sensor includes an array of a plurality of optical sensors.

64. The system of claim 47, wherein the system includes at least one electrical connector configured to communicatively couple an instrument to the system.

65. The system of claim 64, wherein the instrument includes at least one of an thermometer, a hydrometer, a barometer, a photoplethysmograph sensor, a load cell, a biochemical sensor, an optical sensor, a transducer, or a microelectronic machine.

66. The system of claim 47, wherein the system includes at least one first gas connector coupling a first gas supply to the chamber assembly via a first gas circuit.

67. The system of claim 66, wherein the system includes a second gas connector coupling a second gas supply to the chamber assembly via a second gas circuit, the second connector configured to operate independently of the at least one first gas connector.

68. The system of claim 47, wherein the system includes at least one hanger configured to position a source of the media above the chamber assembly.

69. The system of claim 68, wherein the hanger includes a scale configured within the hanger to determine a weight of the source of the media.

70. The system of claim 47, wherein the system includes a bar code reader.

71. The system of claim 47, wherein the system includes a tube welder.

72. The system of claim 47, wherein the system includes an insulative jacket or a conductive jacket at least partially enclosing the chamber assembly.

73. The system of claim 47, wherein an inner surface of the chamber assembly includes a coating or a pattern configured to aid cell mobility or adherence to the inner surface.

74. The system of claim 47, wherein the chamber assembly includes an upper portion removable from a lower portion, the lower portion including the filter.

75. The system of claim 47, wherein the filter includes a coating or a pattern configured to aid cell mobility or adherence to the filter.

76. The system of claim 74, wherein the upper portion includes a gas port at which a gas is received from the first gas circuit.

77. The system of claim 74, wherein the upper portion includes an air diffuser opening and an air diffuser positioned within the air diffuser opening, the air diffuser coupled to the second gas circuit.

78. The system of claim 74, wherein the upper portion includes a spray head opening and a spray head positioned within the spray head opening.

79. The system of claim 78, wherein the spray head includes a gas inlet port coupled to the first gas circuit and a fluid inlet port coupled to a supply of an isotonic aqueous solution including a payload and an alcohol.

80. The system of claim 47, wherein the at least one controller is configured to control one or more of a pressure, a temperature, and a gas composition within the chamber assembly.

81. The system of claim 80, wherein the gas composition includes at least one of carbon dioxide, nitrogen, or oxygen.

82. The system of claim 47, wherein the chamber assembly includes a heating element.

83. The system of claim 47, wherein the system is configured for use as a bioreactor for incubating cells.

84. The system of claim 47, wherein the system is configured for use in a cell cryopreservation process.

85. The system of claim 47, wherein the system is configured for use in a cell permeabilization process.

86. The system of claim 47, wherein the system is configured for use in a cell transduction process.

87. The system of claim 47, wherein the system is configured for use in a cell transfection process.

88. A device for use to deliver a cargo to cells in the absence of alcohol, the device comprising:
a housing including
a base,
at least one controller including circuitry configured to control an operation of the device, and
a display;
one or more fluid circuits including at least one valve, at least one pump, a syringe, and at least one fluid detection sensor;
a chamber assembly received within an articulating frame extending from the front surface of the housing, wherein the chamber assembly is sealed from atmospheric conditions in operation and includes a filter;
at least one media container;
at least one cell culture container fluidically coupled to the chamber assembly via the one or more fluid circuits; and
at least one collection tray configured to receive media or cells.
